SEQUENCE LISTING (566 sequences)

SEQ ID NO: 1 - protein, 398 aa, Homo sapiens
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 2 - DNA, 1312 bp, Homo sapiens
gaattctccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 3 - DNA, 3624 bp, Homo sapiens
cagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgggcctgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggcctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgcttcccggctgcccagagggctggatgcctgccggtccccgagcaagcctgggaactcaggaaaattcacaggacttgggagattctaaatcttaagtgcaattattttaataaaaggggcatttggaatc

SEQ ID NO: 4 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 5 - DNA, 18 bp, Artificial Sequence
  Synthetic oligonucleotide
cgtgacaatacagctgag

SEQ ID NO: 6 - DNA, 18 bp, Artificial Sequence
  Synthetic oligonucleotide
ctcagctgtattgtcacg

SEQ ID NO: 7 - DNA, 22 bp, Artificial Sequence
  Synthetic oligonucleotide
caccgtgacaacgcagctgagg

SEQ ID NO: 8 - DNA, 22 bp, Artificial Sequence
  Synthetic oligonucleotide
cctcagctgcgttgtcacggtg

SEQ ID NO: 9 - DNA, 20 bp, Artificial Sequence
  Synthetic oligonucleotide
ggctgcgcgcctgcgcttcg

SEQ ID NO: 10 - DNA, 20 bp, Artificial Sequence
  Synthetic oligonucleotide
cgaagcgcaggcgcgcagcc

SEQ ID NO: 11 - DNA, 22 bp, Artificial Sequence
  Synthetic oligonucleotide
gcgcttgcgcmtcgcttcctgg

SEQ ID NO: 12 - DNA, 22 bp, Artificial Sequence
  Synthetic oligonucleotide
ccaggaagcgakgcgcaagcgc

SEQ ID NO: 13 - DNA, 24 bp, Artificial Sequence
  Synthetic oligonucleotide
gcttcgcttcccggccctcgtttc

SEQ ID NO: 14 - DNA, 24 bp, Artificial Sequence
  Synthetic oligonucleotide
gaaacgagggccgggaagcgaagc

SEQ ID NO: 15 - DNA, 23 bp, Artificial Sequence
  Synthetic oligonucleotide
ggggctagagtactggacaatgg

SEQ ID NO: 16 - DNA, 23 bp, Artificial Sequence
  Synthetic oligonucleotide
ccattgtccagtactctagcccc

SEQ ID NO: 17 - DNA, 21 bp, Artificial Sequence
  Synthetic oligonucleotide
gctagagcaccggacaatgga

SEQ ID NO: 18 - DNA, 21 bp, Artificial Sequence
  Synthetic oligonucleotide
tccattgtccggtgctctagc

SEQ ID NO: 19 - DNA, 21 bp, Artificial Sequence
  Synthetic oligonucleotide
gctagagcaccggacaatgga

SEQ ID NO: 20 - DNA, 21 bp, Artificial Sequence
  Synthetic oligonucleotide
tccattgtccggtgctctagc

SEQ ID NO: 21 - DNA, 23 bp, Artificial Sequence
  Synthetic oligonucleotide
ctagagcactgtacaatggattg

SEQ ID NO: 22 - DNA, 23 bp, Artificial Sequence
  Synthetic oligonucleotide
caatccattgtacagtgctctag

SEQ ID NO: 23 - DNA, 20 bp, Artificial Sequence
  Synthetic oligonucleotide
gcactggacaaaggattggc

SEQ ID NO: 24 - DNA, 20 bp, Artificial Sequence
  Synthetic oligonucleotide
gccaatcctttgtccagtgc

SEQ ID NO: 25 - DNA, 20 bp, Artificial Sequence
  Synthetic oligonucleotide
gcactggacagtggattggc

SEQ ID NO: 26 - DNA, 20 bp, Artificial Sequence
  Synthetic oligonucleotide
gccaatccactgtccagtgc

SEQ ID NO: 27 - DNA, 21 bp, Artificial Sequence
  Synthetic oligonucleotide
ctggacaatagattggcaagg

SEQ ID NO: 28 - DNA, 26 bp, Artificial Sequence
  Synthetic oligonucleotide
gtcagcaaaattttgccagtgattgc

SEQ ID NO: 29 - DNA, 23 bp, Artificial Sequence
  Synthetic oligonucleotide
gcgaaattttactgacattgatg

SEQ ID NO: 30 - DNA, 23 bp, Artificial Sequence
  Synthetic oligonucleotide
catcaatgtcagtaaaatttcgc

SEQ ID NO: 31 - DNA, 31 bp, Artificial Sequence
  Synthetic oligonucleotide
ggcgaaattttgctaacattgatgattcctg

SEQ ID NO: 32 - DNA, 31 bp, Artificial Sequence
  Synthetic oligonucleotide
caggaatcatcaatgttagcaaaatttcgcc

SEQ ID NO: 33 - DNA, 28 bp, Artificial Sequence
  Synthetic oligonucleotide
cgaaattttgctggcattgatgatattc

SEQ ID NO: 34 - DNA, 28 bp, Artificial Sequence
  Synthetic oligonucleotide
gaatatcatcaatgccagcaaaatttcg

SEQ ID NO: 35 - DNA, 24 bp, Artificial Sequence
  Synthetic oligonucleotide
tgacattgatgagtcctggaaaag

SEQ ID NO: 36 - DNA, 24 bp, Artificial Sequence
  Synthetic oligonucleotide
cttttccaggactcatcaatgtca

SEQ ID NO: 37 - DNA, 26 bp, Artificial Sequence
  Synthetic oligonucleotide
gctgacattgattattcctggaaaag

SEQ ID NO: 38 - DNA, 26 bp, Artificial Sequence
  Synthetic oligonucleotide
cttttccaggaataatcaatgtcagc

SEQ ID NO: 39 - DNA, 34 bp, Artificial Sequence
  Synthetic oligonucleotide
ctgacattgatgattgctggaaaagtataaagag

SEQ ID NO: 40 - DNA, 34 bp, Artificial Sequence
  Synthetic oligonucleotide
ctctttatacttttccagcaatcatcaatgtcag

SEQ ID NO: 41 - DNA, 39 bp, Artificial Sequence
  Synthetic oligonucleotide
gctgacattgatgattcccggaaaagtataaagagtatc

SEQ ID NO: 42 - DNA, 39 bp, Artificial Sequence
  Synthetic oligonucleotide
gatactctttatacttttccgggaatcatcaatgtcagc

SEQ ID NO: 43 - DNA, 23 bp, Artificial Sequence
  Synthetic oligonucleotide
tgatgattccttgaaaagtataa

SEQ ID NO: 44 - DNA, 23 bp, Artificial Sequence
  Synthetic oligonucleotide
ttatacttttcaaggaatcatca

SEQ ID NO: 45 - DNA, 34 bp, Artificial Sequence
  Synthetic oligonucleotide
ctgacattgatgattccttgaaaagtataaagag

SEQ ID NO: 46 - DNA, 34 bp, Artificial Sequence
  Synthetic oligonucleotide
ctctttatacttttcaaggaatcatcaatgtcag

SEQ ID NO: 47 - DNA, 43 bp, Artificial Sequence
  Synthetic oligonucleotide
gctgacattgatgattcctgtaaaagtataaagagtatcttgg

SEQ ID NO: 48 - DNA, 43 bp, Artificial Sequence
  Synthetic oligonucleotide
ccaagatactctttatacttttacaggaatcatcaatgtcagc

SEQ ID NO: 49 - DNA, 21 bp, Artificial Sequence
  Synthetic oligonucleotide
tggaaaagtacaaagagtatc

SEQ ID NO: 50 - DNA, 21 bp, Artificial Sequence
  Synthetic oligonucleotide
gatactctttgtacttttcca

SEQ ID NO: 51 - DNA, 21 bp, Artificial Sequence
  Synthetic oligonucleotide
ccttgccaatctattgtccag

SEQ ID NO: 52 - DNA, 20 bp, Artificial Sequence
  Synthetic oligonucleotide
aatggattggtaaggacgcc

SEQ ID NO: 53 - DNA, 20 bp, Artificial Sequence
  Synthetic oligonucleotide
ggcgtccttaccaatccatt

SEQ ID NO: 54 - DNA, 20 bp, Artificial Sequence
  Synthetic oligonucleotide
gcaaggacgcttaccatggg

SEQ ID NO: 55 - DNA, 20 bp, Artificial Sequence
  Synthetic oligonucleotide
cccatggtaagcgtccttgc

SEQ ID NO: 56 - DNA, 20 bp, Artificial Sequence
  Synthetic oligonucleotide
gcaaggacgtctaccatggg

SEQ ID NO: 57 - DNA, 20 bp, Artificial Sequence
  Synthetic oligonucleotide
cccatggtagacgtccttgc

SEQ ID NO: 58 - DNA, 27 bp, Artificial Sequence
  Synthetic oligonucleotide
aggacgcctaccacgggctggctgcac

SEQ ID NO: 59 - DNA, 27 bp, Artificial Sequence
  Synthetic oligonucleotide
gtgcagccagcccgtggtaggcgtcct

SEQ ID NO: 60 - DNA, 27 bp, Artificial Sequence
  Synthetic oligonucleotide
aggacgcctaccttgggctggctgcac

SEQ ID NO: 61 - DNA, 27 bp, Artificial Sequence
  Synthetic oligonucleotide
gtgcagccagcccaaggtaggcgtcct

SEQ ID NO: 62 - DNA, 25 bp, Artificial Sequence
  Synthetic oligonucleotide
aggacgcctaccgtgggctggctgc

SEQ ID NO: 63 - DNA, 25 bp, Artificial Sequence
  Synthetic oligonucleotide
gcagccagcccacggtaggcgtcct

SEQ ID NO: 64 - DNA, 20 bp, Artificial Sequence
  Synthetic oligonucleotide
ctaccatggwctggctgcac

SEQ ID NO: 65 - DNA, 20 bp, Artificial Sequence
  Synthetic oligonucleotide
gtgcagccagwccatggtag

SEQ ID NO: 66 - DNA, 20 bp, Artificial Sequence
  Synthetic oligonucleotide
ctaccatgcgctggctgcac

SEQ ID NO: 67 - DNA, 20 bp, Artificial Sequence
  Synthetic oligonucleotide
gtgcagccagcgcatggtag

SEQ ID NO: 68 - DNA, 19 bp, Artificial Sequence
  Synthetic oligonucleotide
catgggctgtctgcactgg

SEQ ID NO: 69 - DNA, 19 bp, Artificial Sequence
  Synthetic oligonucleotide
ccagtgcagacagcccatg

SEQ ID NO: 70 - DNA, 21 bp, Artificial Sequence
  Synthetic oligonucleotide
atgggctggcggcactgggag

SEQ ID NO: 71 - DNA, 21 bp, Artificial Sequence
  Synthetic oligonucleotide
ctcccagtgccgccagcccat

SEQ ID NO: 72 - DNA, 17 bp, Artificial Sequence
  Synthetic oligonucleotide
ctggctgcgctgggagc

SEQ ID NO: 73 - DNA, 17 bp, Artificial Sequence
  Synthetic oligonucleotide
gctcccagcgcagccag

SEQ ID NO: 74 - DNA, 17 bp, Artificial Sequence
  Synthetic oligonucleotide
ctggctgtactgggagc

SEQ ID NO: 75 - DNA, 17 bp, Artificial Sequence
  Synthetic oligonucleotide
gctcccagtacagccag

SEQ ID NO: 76 - DNA, 35 bp, Artificial Sequence
  Synthetic oligonucleotide
gattcctggaaaagtacaaagagtatcttggactg

SEQ ID NO: 77 - DNA, 35 bp, Artificial Sequence
  Synthetic oligonucleotide
cagtccaagatactctttgtacttttccaggaatc

SEQ ID NO: 78 - DNA, 26 bp, Artificial Sequence
  Synthetic oligonucleotide
agtataaagagtttcttggactggac

SEQ ID NO: 79 - DNA, 26 bp, Artificial Sequence
  Synthetic oligonucleotide
gtccagtccaagaaactctttatact

SEQ ID NO: 80 - DNA, 23 bp, Artificial Sequence
  Synthetic oligonucleotide
gtataaagagtaacttggactgg

SEQ ID NO: 81 - DNA, 23 bp, Artificial Sequence
  Synthetic oligonucleotide
ccagtccaagttactctttatac

SEQ ID NO: 82 - DNA, 22 bp, Artificial Sequence
  Synthetic oligonucleotide
agagtatcttcgactggacatc

SEQ ID NO: 83 - DNA, 22 bp, Artificial Sequence
  Synthetic oligonucleotide
gatgtccagtcgaagatactct

SEQ ID NO: 84 - DNA, 21 bp, Artificial Sequence
  Synthetic oligonucleotide
gagtatcttgcactggacatc

SEQ ID NO: 85 - DNA, 22 bp, Artificial Sequence
  Synthetic oligonucleotide
gatgtccagtcgaagatactct

SEQ ID NO: 86 - DNA, 30 bp, Artificial Sequence
  Synthetic oligonucleotide
cttggactggacatgttttaaccaggagag

SEQ ID NO: 87 - DNA, 30 bp, Artificial Sequence
  Synthetic oligonucleotide
ctctcctggttaaaacatgtccagtccaag

SEQ ID NO: 88 - DNA, 21 bp, Artificial Sequence
  Synthetic oligonucleotide
ggactggacaccttttaacca

SEQ ID NO: 89 - DNA, 21 bp, Artificial Sequence
  Synthetic oligonucleotide
tggttaaaaggtgtccagtcc

SEQ ID NO: 90 - DNA, 19 bp, Artificial Sequence
  Synthetic oligonucleotide
gttgatgttcctggaccag

SEQ ID NO: 91 - DNA, 19 bp, Artificial Sequence
  Synthetic oligonucleotide
ctggtccaggaacatcaac

SEQ ID NO: 92 - DNA, 19 bp, Artificial Sequence
  Synthetic oligonucleotide
gatgttgctcgaccagggg

SEQ ID NO: 93 - DNA, 19 bp, Artificial Sequence
  Synthetic oligonucleotide
cccctggtcgagcaacatc

SEQ ID NO: 94 - DNA, 25 bp, Artificial Sequence
  Synthetic oligonucleotide
gatgttgctggacgagggggttgga

SEQ ID NO: 95 - DNA, 25 bp, Artificial Sequence
  Synthetic oligonucleotide
tccaaccccctcgtccagcaacatc

SEQ ID NO: 96 - DNA, 21 bp, Artificial Sequence
  Synthetic oligonucleotide
gttgctggactagggggttgg

SEQ ID NO: 97 - DNA, 21 bp, Artificial Sequence
  Synthetic oligonucleotide
ccaaccccctagtccagcaac

SEQ ID NO: 98 - DNA, 22 bp, Artificial Sequence
  Synthetic oligonucleotide
gctggaccagcgggttggaatg

SEQ ID NO: 99 - DNA, 22 bp, Artificial Sequence
  Synthetic oligonucleotide
cattccaacccgctggtccagc

SEQ ID NO: 100 - DNA, 21 bp, Artificial Sequence
  Synthetic oligonucleotide
ggaccaggggattggaatgac

SEQ ID NO: 101 - DNA, 22 bp, Artificial Sequence
  Synthetic oligonucleotide
ctggctgcacggggagcgcttc

SEQ ID NO: 102 - DNA, 22 bp, Artificial Sequence
  Synthetic oligonucleotide
gaagcgctccccgtgcagccag

SEQ ID NO: 103 - DNA, 22 bp, Artificial Sequence
  Synthetic oligonucleotide
ctggctgcacttggagcgcttc

SEQ ID NO: 104 - DNA, 22 bp, Artificial Sequence
  Synthetic oligonucleotide
gaagcgctccaagtgcagccag

SEQ ID NO: 105 - DNA, 22 bp, Artificial Sequence
  Synthetic oligonucleotide
gctgcactggaagcgcttcatg

SEQ ID NO: 106 - DNA, 22 bp, Artificial Sequence
  Synthetic oligonucleotide
catgaagcgcttccagtgcagc

SEQ ID NO: 107 - DNA, 20 bp, Artificial Sequence
  Synthetic oligonucleotide
actgggagcycttcatgtgc

SEQ ID NO: 108 - DNA, 20 bp, Artificial Sequence
  Synthetic oligonucleotide
gcacatgaagrgctcccagt

SEQ ID NO: 109 - DNA, 19 bp, Artificial Sequence
  Synthetic oligonucleotide
cactgggagrgcttcatgt

SEQ ID NO: 110 - DNA, 19 bp, Artificial Sequence
  Synthetic oligonucleotide
acatgaagcyctcccagtg

SEQ ID NO: 111 - DNA, 22 bp, Artificial Sequence
  Synthetic oligonucleotide
ctgggagcgctgcatgtgcaac

SEQ ID NO: 112 - DNA, 22 bp, Artificial Sequence
  Synthetic oligonucleotide
gttgcacatgcagcgctcccag

SEQ ID NO: 113 - DNA, 22 bp, Artificial Sequence
  Synthetic oligonucleotide
gagcgcttcaagtgcaaccttg

SEQ ID NO: 114 - DNA, 22 bp, Artificial Sequence
  Synthetic oligonucleotide
caaggttgcacttgaagcgctc

SEQ ID NO: 115 - DNA, 17 bp, Artificial Sequence
  Synthetic oligonucleotide
gcgcttcatatgcaacc

SEQ ID NO: 116 - DNA, 17 bp, Artificial Sequence
  Synthetic oligonucleotide
ggttgcatatgaagcgc

SEQ ID NO: 117 - DNA, 26 bp, Artificial Sequence
  Synthetic oligonucleotide
gagcgcttcatgtccaaccttgactg

SEQ ID NO: 118 - DNA, 26 bp, Artificial Sequence
  Synthetic oligonucleotide
cagtcaaggttggacatgaagcgctc

SEQ ID NO: 119 - DNA, 21 bp, Artificial Sequence
  Synthetic oligonucleotide
cgcttcatgsgcaaccttgac

SEQ ID NO: 120 - DNA, 21 bp, Artificial Sequence
  Synthetic oligonucleotide
gtcaaggttgcscatgaagcg

SEQ ID NO: 121 - DNA, 20 bp, Artificial Sequence
  Synthetic oligonucleotide
caaccttgacggccaggaag

SEQ ID NO: 122 - DNA, 20 bp, Artificial Sequence
  Synthetic oligonucleotide
cttcctggccgtcaaggttg

SEQ ID NO: 123 - DNA, 22 bp, Artificial Sequence
  Synthetic oligonucleotide
caaccttgactwccaggaagag

SEQ ID NO: 124 - DNA, 22 bp, Artificial Sequence
  Synthetic oligonucleotide
ctcttcctggwagtcaaggttg

SEQ ID NO: 125 - DNA, 29 bp, Artificial Sequence
  Synthetic oligonucleotide
gtgcaaccttgactaccaggaagagccag

SEQ ID NO: 126 - DNA, 21 bp, Artificial Sequence
  Synthetic oligonucleotide
gtcattccaatcccctggtcc

SEQ ID NO: 127 - DNA, 21 bp, Artificial Sequence
  Synthetic oligonucleotide
cagggggttgcaatgacccag

SEQ ID NO: 128 - DNA, 21 bp, Artificial Sequence
  Synthetic oligonucleotide
ctgggtcattgcaaccccctg

SEQ ID NO: 129 - DNA, 19 bp, Artificial Sequence
  Synthetic oligonucleotide
ggggttggagtgacccaga

SEQ ID NO: 130 - DNA, 19 bp, Artificial Sequence
  Synthetic oligonucleotide
tctgggtcactccaacccc

SEQ ID NO: 131 - DNA, 21 bp, Artificial Sequence
  Synthetic oligonucleotide
ggttggaatgtcccagatatg

SEQ ID NO: 132 - DNA, 21 bp, Artificial Sequence
  Synthetic oligonucleotide
catatctgggacattccaacc

SEQ ID NO: 133 - DNA, 22 bp, Artificial Sequence
  Synthetic oligonucleotide
gggttggaattacccagatatg

SEQ ID NO: 134 - DNA, 22 bp, Artificial Sequence
  Synthetic oligonucleotide
catatctgggtaattccaaccc

SEQ ID NO: 135 - DNA, 21 bp, Artificial Sequence
  Synthetic oligonucleotide
tggaatgaccgagatatgtta

SEQ ID NO: 136 - DNA, 21 bp, Artificial Sequence
  Synthetic oligonucleotide
taacatatctcggtcattcca

SEQ ID NO: 137 - DNA, 23 bp, Artificial Sequence
  Synthetic oligonucleotide
ttggaatgacctagatatgttag

SEQ ID NO: 138 - DNA, 23 bp, Artificial Sequence
  Synthetic oligonucleotide
ctaacatatctaggtcattccaa

SEQ ID NO: 139 - DNA, 23 bp, Artificial Sequence
  Synthetic oligonucleotide
ggaatgacccamatatgttagtg

SEQ ID NO: 140 - DNA, 22 bp, Artificial Sequence
  Synthetic oligonucleotide
cactaacatattgggtcattcc

SEQ ID NO: 141 - DNA, 32 bp, Artificial Sequence
  Synthetic oligonucleotide
ggttggaatgacccacatatgttagtgattgg

SEQ ID NO: 142 - DNA, 32 bp, Artificial Sequence
  Synthetic oligonucleotide
ccaatcactaacatatgtgggtcattccaacc

SEQ ID NO: 143 - DNA, 22 bp, Artificial Sequence
  Synthetic oligonucleotide
gaatgacccagttatgttagtg

SEQ ID NO: 144 - DNA, 22 bp, Artificial Sequence
  Synthetic oligonucleotide
cactaacataactgggtcattc

SEQ ID NO: 145 - DNA, 21 bp, Artificial Sequence
  Synthetic oligonucleotide
atgacccagaaatgttagtga

SEQ ID NO: 146 - DNA, 21 bp, Artificial Sequence
  Synthetic oligonucleotide
tcactaacatttctgggtcat

SEQ ID NO: 147 - DNA, 22 bp, Artificial Sequence
  Synthetic oligonucleotide
gacccagataggttagtgattg

SEQ ID NO: 148 - DNA, 22 bp, Artificial Sequence
  Synthetic oligonucleotide
caatcactaacctatctgggtc

SEQ ID NO: 149 - DNA, 23 bp, Artificial Sequence
  Synthetic oligonucleotide
gacccagatatattagtgattgg

SEQ ID NO: 150 - DNA, 23 bp, Artificial Sequence
  Synthetic oligonucleotide
ccaatcactaatatatctgggtc

SEQ ID NO: 151 - DNA, 29 bp, Artificial Sequence
  Synthetic oligonucleotide
ctggctcttcctggtagtcaaggttgcac

SEQ ID NO: 152 - DNA, 28 bp, Artificial Sequence
  Synthetic oligonucleotide
gagccagattcctacatcagtgagaagc

SEQ ID NO: 153 - DNA, 28 bp, Artificial Sequence
  Synthetic oligonucleotide
gcttctcactgatgtaggaatctggctc

SEQ ID NO: 154 - DNA, 21 bp, Artificial Sequence
  Synthetic oligonucleotide
tcctgcatcactgagaagctc

SEQ ID NO: 155 - DNA, 21 bp, Artificial Sequence
  Synthetic oligonucleotide
gagcttctcagtgatgcagga

SEQ ID NO: 156 - DNA, 22 bp, Artificial Sequence
  Synthetic oligonucleotide
ctgcatcagtaagaagctcttc

SEQ ID NO: 157 - DNA, 22 bp, Artificial Sequence
  Synthetic oligonucleotide
gaagagcttcttactgatgcag

SEQ ID NO: 158 - DNA, 22 bp, Artificial Sequence
  Synthetic oligonucleotide
ctgcatcagtgggaagctcttc

SEQ ID NO: 159 - DNA, 22 bp, Artificial Sequence
  Synthetic oligonucleotide
gaagagcttcccactgatgcag

SEQ ID NO: 160 - DNA, 20 bp, Artificial Sequence
  Synthetic oligonucleotide
cagtgagaagttcttcatgg

SEQ ID NO: 161 - DNA, 20 bp, Artificial Sequence
  Synthetic oligonucleotide
ccaggaagaacttctcactg

SEQ ID NO: 162 - DNA, 29 bp, Artificial Sequence
  Synthetic oligonucleotide
gcatcagtgagaagttcttcatggagatg

SEQ ID NO: 163 - DNA, 29 bp, Artificial Sequence
  Synthetic oligonucleotide
catctccatgaagaacttctcactgatgc

SEQ ID NO: 164 - DNA, 22 bp, Artificial Sequence
  Synthetic oligonucleotide
cttcatggagagggcagagctc

SEQ ID NO: 165 - DNA, 22 bp, Artificial Sequence
  Synthetic oligonucleotide
gagctctgccctctccatgaag

SEQ ID NO: 166 - DNA, 22 bp, Artificial Sequence
  Synthetic oligonucleotide
cttcatggagatagcagagctc

SEQ ID NO: 167 - DNA, 22 bp, Artificial Sequence
  Synthetic oligonucleotide
gagctctgctatctccatgaag

SEQ ID NO: 168 - DNA, 22 bp, Artificial Sequence
  Synthetic oligonucleotide
catggagatggtagagctcatg

SEQ ID NO: 169 - DNA, 22 bp, Artificial Sequence
  Synthetic oligonucleotide
catgagctctaccatctccatg

SEQ ID NO: 170 - DNA, 22 bp, Artificial Sequence
  Synthetic oligonucleotide
gcagagctcagggtctcagaag

SEQ ID NO: 171 - DNA, 22 bp, Artificial Sequence
  Synthetic oligonucleotide
cttctgagaccctgagctctgc

SEQ ID NO: 172 - DNA, 25 bp, Artificial Sequence
  Synthetic oligonucleotide
ctcagaaggctgtaaggatgcaggt

SEQ ID NO: 173 - DNA, 25 bp, Artificial Sequence
  Synthetic oligonucleotide
acctgcatccttacagccttctgag

SEQ ID NO: 174 - DNA, 22 bp, Artificial Sequence
  Synthetic oligonucleotide
ctcagaaggctcgaaggatgca

SEQ ID NO: 175 - DNA, 22 bp, Artificial Sequence
  Synthetic oligonucleotide
tgcatccttcgagccttctgag

SEQ ID NO: 176 - DNA, 21 bp, Artificial Sequence
  Synthetic oligonucleotide
ccagatatgtcagtgattggc

SEQ ID NO: 177 - DNA, 21 bp, Artificial Sequence
  Synthetic oligonucleotide
gccaatcactgacatatctgg

SEQ ID NO: 178 - DNA, 21 bp, Artificial Sequence
  Synthetic oligonucleotide
gatatgttagcgattggcaac

SEQ ID NO: 179 - DNA, 21 bp, Artificial Sequence
  Synthetic oligonucleotide
gttgccaatcgctaacatatc

SEQ ID NO: 180 - DNA, 23 bp, Artificial Sequence
  Synthetic oligonucleotide
cagatatgttaatgattggcaac

SEQ ID NO: 181 - DNA, 23 bp, Artificial Sequence
  Synthetic oligonucleotide
gttgccaatcattaacatatctg

SEQ ID NO: 182 - DNA, 23 bp, Artificial Sequence
  Synthetic oligonucleotide
tatgttagtgactggcaactttg

SEQ ID NO: 183 - DNA, 23 bp, Artificial Sequence
  Synthetic oligonucleotide
caaagttgccagtcactaacata

SEQ ID NO: 184 - DNA, 19 bp, Artificial Sequence
  Synthetic oligonucleotide
ttagtgattgtcaactttg

SEQ ID NO: 185 - DNA, 19 bp, Artificial Sequence
  Synthetic oligonucleotide
caaagttgacaatcactaa

SEQ ID NO: 186 - DNA, 21 bp, Artificial Sequence
  Synthetic oligonucleotide
gttagtgatttgcaactttgg

SEQ ID NO: 187 - DNA, 21 bp, Artificial Sequence
  Synthetic oligonucleotide
ccaaagttgcaaatcactaac

SEQ ID NO: 188 - DNA, 23 bp, Artificial Sequence
  Synthetic oligonucleotide
tgttagtgattagcaactttggc

SEQ ID NO: 189 - DNA, 23 bp, Artificial Sequence
  Synthetic oligonucleotide
gccaaagttgctaatcactaaca

SEQ ID NO: 190 - DNA, 23 bp, Artificial Sequence
  Synthetic oligonucleotide
gtgattggcaaatttggcctcag

SEQ ID NO: 191 - DNA, 23 bp, Artificial Sequence
  Synthetic oligonucleotide
ctgaggccaaatttgccaatcac

SEQ ID NO: 192 - DNA, 21 bp, Artificial Sequence
  Synthetic oligonucleotide
gtgattggcagctttggcctc

SEQ ID NO: 193 - DNA, 21 bp, Artificial Sequence
  Synthetic oligonucleotide
gaggccaaagctgccaatcac

SEQ ID NO: 194 - DNA, 25 bp, Artificial Sequence
  Synthetic oligonucleotide
caactttggcctcggctggaatcag

SEQ ID NO: 195 - DNA, 25 bp, Artificial Sequence
  Synthetic oligonucleotide
ctgattccagccgaggccaaagttg

SEQ ID NO: 196 - DNA, 23 bp, Artificial Sequence
  Synthetic oligonucleotide
ctttggcctcaactggaatcagc

SEQ ID NO: 197 - DNA, 23 bp, Artificial Sequence
  Synthetic oligonucleotide
gctgattccagttgaggccaaag

SEQ ID NO: 198 - DNA, 22 bp, Artificial Sequence
  Synthetic oligonucleotide
agctggaatcggcaagtaactc

SEQ ID NO: 199 - DNA, 22 bp, Artificial Sequence
  Synthetic oligonucleotide
gagttacttgccgattccagct

SEQ ID NO: 200 - DNA, 23 bp, Artificial Sequence
  Synthetic oligonucleotide
cagctggaatcaccaagtaactc

SEQ ID NO: 201 - DNA, 22 bp, Artificial Sequence
  Synthetic oligonucleotide
gaaggatgcagattatgagtac

SEQ ID NO: 202 - DNA, 22 bp, Artificial Sequence
  Synthetic oligonucleotide
gtactcataatctgcatccttc

SEQ ID NO: 203 - DNA, 25 bp, Artificial Sequence
  Synthetic oligonucleotide
ggatgcaggttgtgagtacctctgc

SEQ ID NO: 204 - DNA, 25 bp, Artificial Sequence
  Synthetic oligonucleotide
gcagaggtactcacaacctgcatcc

SEQ ID NO: 205 - DNA, 22 bp, Artificial Sequence
  Synthetic oligonucleotide
ggttatgaggacctctgcattg

SEQ ID NO: 206 - DNA, 22 bp, Artificial Sequence
  Synthetic oligonucleotide
caatgcagaggtcctcataacc

SEQ ID NO: 207 - DNA, 24 bp, Artificial Sequence
  Synthetic oligonucleotide
gttatgagtaccsctgcattgatg

SEQ ID NO: 208 - DNA, 24 bp, Artificial Sequence
  Synthetic oligonucleotide
catcaatgcagsggtactcataac

SEQ ID NO: 209 - DNA, 21 bp, Artificial Sequence
  Synthetic oligonucleotide
cctctgcatthatgactgttg

SEQ ID NO: 210 - DNA, 21 bp, Artificial Sequence
  Synthetic oligonucleotide
caacagtcatdaatgcagagg

SEQ ID NO: 211 - DNA, 27 bp, Artificial Sequence
  Synthetic oligonucleotide
tacctctgcattaatgactgttggatg

SEQ ID NO: 212 - DNA, 27 bp, Artificial Sequence
  Synthetic oligonucleotide
catccaacagtcattaatgcagaggta

SEQ ID NO: 213 - DNA, 27 bp, Artificial Sequence
  Synthetic oligonucleotide
tacctctgcattcatgactgttggatg

SEQ ID NO: 214 - DNA, 27 bp, Artificial Sequence
  Synthetic oligonucleotide
catccaacagtcatgaatgcagaggta

SEQ ID NO: 215 - DNA, 34 bp, Artificial Sequence
  Synthetic oligonucleotide
gagtacctctgcatttatgactgttggatggctc

SEQ ID NO: 216 - DNA, 34 bp, Artificial Sequence
  Synthetic oligonucleotide
gagccatccaacagtcataaatgcagaggtactc

SEQ ID NO: 217 - DNA, 22 bp, Artificial Sequence
  Synthetic oligonucleotide
ctgcattgatggctgttggatg

SEQ ID NO: 218 - DNA, 22 bp, Artificial Sequence
  Synthetic oligonucleotide
catccaacagccatcaatgcag

SEQ ID NO: 219 - DNA, 22 bp, Artificial Sequence
  Synthetic oligonucleotide
ctgcattgatgtctgttggatg

SEQ ID NO: 220 - DNA, 22 bp, Artificial Sequence
  Synthetic oligonucleotide
catccaacagacatcaatgcag

SEQ ID NO: 221 - DNA, 22 bp, Artificial Sequence
  Synthetic oligonucleotide
ctgcattgataactgttggatg

SEQ ID NO: 222 - DNA, 22 bp, Artificial Sequence
  Synthetic oligonucleotide
catccaacagttatcaatgcag

SEQ ID NO: 223 - DNA, 24 bp, Artificial Sequence
  Synthetic oligonucleotide
gcattgatgactcttggatggctc

SEQ ID NO: 224 - DNA, 24 bp, Artificial Sequence
  Synthetic oligonucleotide
gagccatccaagagtcatcaatgc

SEQ ID NO: 225 - DNA, 23 bp, Artificial Sequence
  Synthetic oligonucleotide
gagttacttggtgattccagctg

SEQ ID NO: 226 - DNA, 21 bp, Artificial Sequence
  Synthetic oligonucleotide
cagctggaataagcaagtaac

SEQ ID NO: 227 - DNA, 21 bp, Artificial Sequence
  Synthetic oligonucleotide
gttacttgcttattccagctg

SEQ ID NO: 228 - DNA, 21 bp, Artificial Sequence
  Synthetic oligonucleotide
ggaatcagcatgtaactcaga

SEQ ID NO: 229 - DNA, 21 bp, Artificial Sequence
  Synthetic oligonucleotide
tctgagttacatgctgattcc

SEQ ID NO: 230 - DNA, 22 bp, Artificial Sequence
  Synthetic oligonucleotide
ctggaatcagaaagtaactcag

SEQ ID NO: 231 - DNA, 21 bp, Artificial Sequence
  Synthetic oligonucleotide
ctgagttactttctgattcca

SEQ ID NO: 232 - DNA, 21 bp, Artificial Sequence
  Synthetic oligonucleotide
cagcaagtaaatcagatggcc

SEQ ID NO: 233 - DNA, 21 bp, Artificial Sequence
  Synthetic oligonucleotide
ggccatctgatttacttgctg

SEQ ID NO: 234 - DNA, 21 bp, Artificial Sequence
  Synthetic oligonucleotide
caagtaactccgatggccctc

SEQ ID NO: 235 - DNA, 21 bp, Artificial Sequence
  Synthetic oligonucleotide
gagggccatcggagttacttg

SEQ ID NO: 236 - DNA, 20 bp, Artificial Sequence
  Synthetic oligonucleotide
gtaactcagacggccctctg

SEQ ID NO: 237 - DNA, 20 bp, Artificial Sequence
  Synthetic oligonucleotide
cagagggccgtctgagttac

SEQ ID NO: 238 - DNA, 23 bp, Artificial Sequence
  Synthetic oligonucleotide
taactcagatgcccctctgggct

SEQ ID NO: 239 - DNA, 23 bp, Artificial Sequence
  Synthetic oligonucleotide
agcccagaggggcatctgagtta

SEQ ID NO: 240 - DNA, 22 bp, Artificial Sequence
  Synthetic oligonucleotide
aactcagatggacctctgggct

SEQ ID NO: 241 - DNA, 22 bp, Artificial Sequence
  Synthetic oligonucleotide
agcccagaggtccatctgagtt

SEQ ID NO: 242 - DNA, 21 bp, Artificial Sequence
  Synthetic oligonucleotide
gatggccctcggggctatcat

SEQ ID NO: 243 - DNA, 21 bp, Artificial Sequence
  Synthetic oligonucleotide
atgatagccccgagggccatc

SEQ ID NO: 244 - DNA, 21 bp, Artificial Sequence
  Synthetic oligonucleotide
atggccctctgtgctatcatg

SEQ ID NO: 245 - DNA, 21 bp, Artificial Sequence
  Synthetic oligonucleotide
catgatagcacagagggccat

SEQ ID NO: 246 - DNA, 22 bp, Artificial Sequence
  Synthetic oligonucleotide
gccctctgggatatcatggctg

SEQ ID NO: 247 - DNA, 22 bp, Artificial Sequence
  Synthetic oligonucleotide
cagccatgatatcccagagggc

SEQ ID NO: 248 - DNA, 19 bp, Artificial Sequence
  Synthetic oligonucleotide
gccctctggcctatcatgg

SEQ ID NO: 249 - DNA, 24 bp, Artificial Sequence
  Synthetic oligonucleotide
gcattgatgactattggatggctc

SEQ ID NO: 250 - DNA, 24 bp, Artificial Sequence
  Synthetic oligonucleotide
gagccatccaatagtcatcaatgc

SEQ ID NO: 251 - DNA, 21 bp, Artificial Sequence
  Synthetic oligonucleotide
gatgactgttcgatggctccc

SEQ ID NO: 252 - DNA, 21 bp, Artificial Sequence
  Synthetic oligonucleotide
gggagccatcgaacagtcatc

SEQ ID NO: 253 - DNA, 23 bp, Artificial Sequence
  Synthetic oligonucleotide
ctgttggatgcctccccaaagag

SEQ ID NO: 254 - DNA, 23 bp, Artificial Sequence
  Synthetic oligonucleotide
ctctttggggaggcatccaacag

SEQ ID NO: 255 - DNA, 22 bp, Artificial Sequence
  Synthetic oligonucleotide
gctccccaaamagattcagaag

SEQ ID NO: 256 - DNA, 22 bp, Artificial Sequence
  Synthetic oligonucleotide
cttctgaatctktttggggagc

SEQ ID NO: 257 - DNA, 24 bp, Artificial Sequence
  Synthetic oligonucleotide
ggctccccaaacagattcagaagg

SEQ ID NO: 258 - DNA, 24 bp, Artificial Sequence
  Synthetic oligonucleotide
ccttctgaatctgtttggggagcc

SEQ ID NO: 259 - DNA, 25 bp, Artificial Sequence
  Synthetic oligonucleotide
caaagagattcacaaggcagacttc

SEQ ID NO: 260 - DNA, 25 bp, Artificial Sequence
  Synthetic oligonucleotide
gaagtctgccttgtgaatctctttg

SEQ ID NO: 261 - DNA, 25 bp, Artificial Sequence
  Synthetic oligonucleotide
gcagaccctcagagctttcctcatg

SEQ ID NO: 262 - DNA, 25 bp, Artificial Sequence
  Synthetic oligonucleotide
catgaggaaagctctgagggtctgc

SEQ ID NO: 263 - DNA, 24 bp, Artificial Sequence
  Synthetic oligonucleotide
cagaccctcagtgctttcctcatg

SEQ ID NO: 264 - DNA, 24 bp, Artificial Sequence
  Synthetic oligonucleotide
catgaggaaagcactgagggtctg

SEQ ID NO: 265 - DNA, 20 bp, Artificial Sequence
  Synthetic oligonucleotide
ccctcagcgctctcctcatg

SEQ ID NO: 266 - DNA, 20 bp, Artificial Sequence
  Synthetic oligonucleotide
catgaggagagcgctgaggg

SEQ ID NO: 267 - DNA, 22 bp, Artificial Sequence
  Synthetic oligonucleotide
ctcatgggatttgccagctagc

SEQ ID NO: 268 - DNA, 22 bp, Artificial Sequence
  Synthetic oligonucleotide
gctagctggcaaatcccatgag

SEQ ID NO: 269 - DNA, 23 bp, Artificial Sequence
  Synthetic oligonucleotide
gattcgccagccagctaattatg

SEQ ID NO: 270 - DNA, 23 bp, Artificial Sequence
  Synthetic oligonucleotide
cataattagctggctggcgaatc

SEQ ID NO: 271 - DNA, 28 bp, Artificial Sequence
  Synthetic oligonucleotide
tcgccagctamctaattatgttcacagc

SEQ ID NO: 272 - DNA, 28 bp, Artificial Sequence
  Synthetic oligonucleotide
gctgtgaacataattagktagctggcga

SEQ ID NO: 273 - DNA, 29 bp, Artificial Sequence
  Synthetic oligonucleotide
gctagctaattatgatcacagcaaaggac

SEQ ID NO: 274 - DNA, 19 bp, Artificial Sequence
  Synthetic oligonucleotide
ccatgataggccagagggc

SEQ ID NO: 275 - DNA, 25 bp, Artificial Sequence
  Synthetic oligonucleotide
ctctgggctttcatggctgctcctt

SEQ ID NO: 276 - DNA, 25 bp, Artificial Sequence
  Synthetic oligonucleotide
aaggagcagccatgaaagcccagag

SEQ ID NO: 277 - DNA, 21 bp, Artificial Sequence
  Synthetic oligonucleotide
gggctatcatcgctgctcctt

SEQ ID NO: 278 - DNA, 21 bp, Artificial Sequence
  Synthetic oligonucleotide
aaggagcagcgatgatagccc

SEQ ID NO: 279 - DNA, 23 bp, Artificial Sequence
  Synthetic oligonucleotide
ctatcatggctcctcctttattc

SEQ ID NO: 280 - DNA, 23 bp, Artificial Sequence
  Synthetic oligonucleotide
gaataaaggaggagccatgatag

SEQ ID NO: 281 - DNA, 22 bp, Artificial Sequence
  Synthetic oligonucleotide
catggctgctgctttattcatg

SEQ ID NO: 282 - DNA, 22 bp, Artificial Sequence
  Synthetic oligonucleotide
catgaataaagcagcagccatg

SEQ ID NO: 283 - DNA, 22 bp, Artificial Sequence
  Synthetic oligonucleotide
catggctgctwctttattcatg

SEQ ID NO: 284 - DNA, 22 bp, Artificial Sequence
  Synthetic oligonucleotide
catgaataaagwagcagccatg

SEQ ID NO: 285 - DNA, 28 bp, Artificial Sequence
  Synthetic oligonucleotide
gctgctcctttatgcatgtctaatgacc

SEQ ID NO: 286 - DNA, 28 bp, Artificial Sequence
  Synthetic oligonucleotide
ggtcattagacatgcataaaggagcagc

SEQ ID NO: 287 - DNA, 25 bp, Artificial Sequence
  Synthetic oligonucleotide
ctttattcatgtktaatgacctccg

SEQ ID NO: 288 - DNA, 25 bp, Artificial Sequence
  Synthetic oligonucleotide
cggaggtcattamacatgaataaag

SEQ ID NO: 289 - DNA, 23 bp, Artificial Sequence
  Synthetic oligonucleotide
attcatgtctagtgacctccgac

SEQ ID NO: 290 - DNA, 23 bp, Artificial Sequence
  Synthetic oligonucleotide
gtcggaggtcactagacatgaat

SEQ ID NO: 291 - DNA, 24 bp, Artificial Sequence
  Synthetic oligonucleotide
tattcatgtctaaggacctccgac

SEQ ID NO: 292 - DNA, 24 bp, Artificial Sequence
  Synthetic oligonucleotide
gtcggaggtccttagacatgaata

SEQ ID NO: 293 - DNA, 24 bp, Artificial Sequence
  Synthetic oligonucleotide
tattcatgtctcatgacctccgac

SEQ ID NO: 294 - DNA, 24 bp, Artificial Sequence
  Synthetic oligonucleotide
gtcggaggtcatgagacatgaata

SEQ ID NO: 295 - DNA, 26 bp, Artificial Sequence
  Synthetic oligonucleotide
tcatgtctaatggcctccgacacatc

SEQ ID NO: 296 - DNA, 26 bp, Artificial Sequence
  Synthetic oligonucleotide
gatgtgtcggaggccattagacatga

SEQ ID NO: 297 - DNA, 24 bp, Artificial Sequence
  Synthetic oligonucleotide
gtctaatgacccccgacacatcag

SEQ ID NO: 298 - DNA, 24 bp, Artificial Sequence
  Synthetic oligonucleotide
ctgatgtgtcgggggtcattagac

SEQ ID NO: 299 - DNA, 29 bp, Artificial Sequence
  Synthetic oligonucleotide
gtcctttgctgtgatcataattagctagc

SEQ ID NO: 300 - DNA, 22 bp, Artificial Sequence
  Synthetic oligonucleotide
cacagcaaagaactgaagctag

SEQ ID NO: 301 - DNA, 22 bp, Artificial Sequence
  Synthetic oligonucleotide
ctagcttcagttctttgctgtg

SEQ ID NO: 302 - DNA, 21 bp, Artificial Sequence
  Synthetic oligonucleotide
cagcaaaggaccgaagctagg

SEQ ID NO: 303 - DNA, 24 bp, Artificial Sequence
  Synthetic oligonucleotide
atccctagcttcggtcctttgctg

SEQ ID NO: 304 - DNA, 24 bp, Artificial Sequence
  Synthetic oligonucleotide
aggactgaagccagggatttatgc

SEQ ID NO: 305 - DNA, 24 bp, Artificial Sequence
  Synthetic oligonucleotide
gcataaatccctggcttcagtcct

SEQ ID NO: 306 - DNA, 28 bp, Artificial Sequence
  Synthetic oligonucleotide
ggactgaagctagagatttatgcagatg

SEQ ID NO: 307 - DNA, 28 bp, Artificial Sequence
  Synthetic oligonucleotide
catctgcataaatctctagcttcagtcc

SEQ ID NO: 308 - DNA, 27 bp, Artificial Sequence
  Synthetic oligonucleotide
gactgaagctaaggatttatgcagatg

SEQ ID NO: 309 - DNA, 27 bp, Artificial Sequence
  Synthetic oligonucleotide
catctgcataaatccttagcttcagtc

SEQ ID NO: 310 - DNA, 24 bp, Artificial Sequence
  Synthetic oligonucleotide
gctagggatttctgcagatgttgg

SEQ ID NO: 311 - DNA, 24 bp, Artificial Sequence
  Synthetic oligonucleotide
ccaacatctgcagaaatccctagc

SEQ ID NO: 312 - DNA, 25 bp, Artificial Sequence
  Synthetic oligonucleotide
gctagggatttatgtagatgttgga

SEQ ID NO: 313 - DNA, 25 bp, Artificial Sequence
  Synthetic oligonucleotide
tccaacatctacataaatccctagc

SEQ ID NO: 314 - DNA, 21 bp, Artificial Sequence
  Synthetic oligonucleotide
ggatttatgcacatgttggaa

SEQ ID NO: 315 - DNA, 21 bp, Artificial Sequence
  Synthetic oligonucleotide
ttccaacatgtgcataaatcc

SEQ ID NO: 316 - DNA, 30 bp, Artificial Sequence
  Synthetic oligonucleotide
gggatttatgcacatgttggaaataaaacc

SEQ ID NO: 317 - DNA, 30 bp, Artificial Sequence
  Synthetic oligonucleotide
ggttttatttccaacatgtgcataaatccc

SEQ ID NO: 318 - DNA, 24 bp, Artificial Sequence
  Synthetic oligonucleotide
tgcagatgttgaaaataaaacctg

SEQ ID NO: 319 - DNA, 24 bp, Artificial Sequence
  Synthetic oligonucleotide
caggttttattttcaacatctgca

SEQ ID NO: 320 - DNA, 24 bp, Artificial Sequence
  Synthetic oligonucleotide
tgcagatgttagaaataaaacctg

SEQ ID NO: 321 - DNA, 24 bp, Artificial Sequence
  Synthetic oligonucleotide
caggttttatttctaacatctgca

SEQ ID NO: 322 - DNA, 37 bp, Artificial Sequence
  Synthetic oligonucleotide
ggatttatgcagatgttgaaaataaaacctgcgcagc

SEQ ID NO: 323 - DNA, 37 bp, Artificial Sequence
  Synthetic oligonucleotide
gctgcgcaggttttattttcaacatctgcataaatcc

SEQ ID NO: 324 - DNA, 22 bp, Artificial Sequence
  Synthetic oligonucleotide
gtctaatgacttccgacacatc

SEQ ID NO: 325 - DNA, 22 bp, Artificial Sequence
  Synthetic oligonucleotide
gatgtgtcggaagtcattagac

SEQ ID NO: 326 - DNA, 22 bp, Artificial Sequence
  Synthetic oligonucleotide
gtctaatgaccaccgacacatc

SEQ ID NO: 327 - DNA, 22 bp, Artificial Sequence
  Synthetic oligonucleotide
gatgtgtcggtggtcattagac

SEQ ID NO: 328 - DNA, 23 bp, Artificial Sequence
  Synthetic oligonucleotide
ctaatgacctcggacacatcagc

SEQ ID NO: 329 - DNA, 23 bp, Artificial Sequence
  Synthetic oligonucleotide
gctgatgtgtccgaggtcattag

SEQ ID NO: 330 - DNA, 23 bp, Artificial Sequence
  Synthetic oligonucleotide
ctaatgacctcccacacatcagc

SEQ ID NO: 331 - DNA, 23 bp, Artificial Sequence
  Synthetic oligonucleotide
gctgatgtgtgggaggtcattag

SEQ ID NO: 332 - DNA, 23 bp, Artificial Sequence
  Synthetic oligonucleotide
ctccgacacaacagccctcaagc

SEQ ID NO: 333 - DNA, 23 bp, Artificial Sequence
  Synthetic oligonucleotide
gcttgagggctgttgtgtcggag

SEQ ID NO: 334 - DNA, 20 bp, Artificial Sequence
  Synthetic oligonucleotide
gccaaagctttccttcagga

SEQ ID NO: 335 - DNA, 20 bp, Artificial Sequence
  Synthetic oligonucleotide
tcctgaaggaaagctttggc

SEQ ID NO: 336 - DNA, 22 bp, Artificial Sequence
  Synthetic oligonucleotide
gctctccttcacgataaggacg

SEQ ID NO: 337 - DNA, 22 bp, Artificial Sequence
  Synthetic oligonucleotide
cgtccttatcgtgaaggagagc

SEQ ID NO: 338 - DNA, 21 bp, Artificial Sequence
  Synthetic oligonucleotide
ctctccttcagtataaggacg

SEQ ID NO: 339 - DNA, 21 bp, Artificial Sequence
  Synthetic oligonucleotide
cgtccttatactgaaggagag

SEQ ID NO: 340 - DNA, 21 bp, Artificial Sequence
  Synthetic oligonucleotide
gataaggacgaaattgccatc

SEQ ID NO: 341 - DNA, 21 bp, Artificial Sequence
  Synthetic oligonucleotide
gatggcaatttcgtccttatc

SEQ ID NO: 342 - DNA, 22 bp, Artificial Sequence
  Synthetic oligonucleotide
aaggacgtaamtgccatcaatc

SEQ ID NO: 343 - DNA, 22 bp, Artificial Sequence
  Synthetic oligonucleotide
gattgatggcakttacgtcctt

SEQ ID NO: 344 - DNA, 22 bp, Artificial Sequence
  Synthetic oligonucleotide
aattgccatcattcaggacccc

SEQ ID NO: 345 - DNA, 22 bp, Artificial Sequence
  Synthetic oligonucleotide
ggggtcctgaatgatggcaatt

SEQ ID NO: 346 - DNA, 22 bp, Artificial Sequence
  Synthetic oligonucleotide
aattgccatcaagcaggacccc

SEQ ID NO: 347 - DNA, 22 bp, Artificial Sequence
  Synthetic oligonucleotide
ggggtcctgcttgatggcaatt

SEQ ID NO: 348 - DNA, 22 bp, Artificial Sequence
  Synthetic oligonucleotide
aattgccatctatcaggacccc

SEQ ID NO: 349 - DNA, 37 bp, Artificial Sequence
  Synthetic oligonucleotide
ggatttatgcagatgttcgaaataaaacctgcgcagc

SEQ ID NO: 350 - DNA, 37 bp, Artificial Sequence; identical to SEQ ID NO: 349
  Synthetic oligonucleotide

SEQ ID NO: 351 - DNA, 22 bp, Artificial Sequence
  Synthetic oligonucleotide
ggaaataaaatctgcgcaggct

SEQ ID NO: 352 - DNA, 22 bp, Artificial Sequence
  Synthetic oligonucleotide
agcctgcgcagattttatttcc

SEQ ID NO: 353 - DNA, 24 bp, Artificial Sequence
  Synthetic oligonucleotide
ggaaataaaacccgcgcaggcttc

SEQ ID NO: 354 - DNA, 24 bp, Artificial Sequence
  Synthetic oligonucleotide
gaagcctgcgcgggttttatttcc

SEQ ID NO: 355 - DNA, 24 bp, Artificial Sequence
  Synthetic oligonucleotide
gaaataaaacctrcgcaggcttcc

SEQ ID NO: 356 - DNA, 24 bp, Artificial Sequence
  Synthetic oligonucleotide
ggaagcctgcgyaggttttatttc

SEQ ID NO: 357 - DNA, 28 bp, Artificial Sequence
  Synthetic oligonucleotide
ggaaataaaacctgggcaggcttccctg

SEQ ID NO: 358 - DNA, 28 bp, Artificial Sequence
  Synthetic oligonucleotide
cagggaagcctgcccaggttttatttcc

SEQ ID NO: 359 - DNA, 25 bp, Artificial Sequence
  Synthetic oligonucleotide
gaaataaaacctgcacaggcttccc

SEQ ID NO: 360 - DNA, 25 bp, Artificial Sequence
  Synthetic oligonucleotide
gggaagcctgtgcaggttttatttc

SEQ ID NO: 361 - DNA, 22 bp, Artificial Sequence
  Synthetic oligonucleotide
ataaaacctgcccaggcttccc

SEQ ID NO: 362 - DNA, 22 bp, Artificial Sequence
  Synthetic oligonucleotide
gggaagcctgggcaggttttat

SEQ ID NO: 363 - DNA, 19 bp, Artificial Sequence
  Synthetic oligonucleotide
cctgcgcagtcttccctgg

SEQ ID NO: 364 - DNA, 19 bp, Artificial Sequence
  Synthetic oligonucleotide
ccagggaagactgcgcagg

SEQ ID NO: 365 - DNA, 20 bp, Artificial Sequence
  Synthetic oligonucleotide
ggcttccctaggagttttgg

SEQ ID NO: 366 - DNA, 20 bp, Artificial Sequence
  Synthetic oligonucleotide
ccaaaactcctagggaagcc

SEQ ID NO: 367 - DNA, 21 bp, Artificial Sequence
  Synthetic oligonucleotide
ttccctgggaattttggatac

SEQ ID NO: 368 - DNA, 21 bp, Artificial Sequence
  Synthetic oligonucleotide
gtatccaaaattcccagggaa

SEQ ID NO: 369 - DNA, 20 bp, Artificial Sequence
  Synthetic oligonucleotide
ccctgggaggtttggatact

SEQ ID NO: 370 - DNA, 20 bp, Artificial Sequence
  Synthetic oligonucleotide
agtatccaaacctcccaggg

SEQ ID NO: 371 - DNA, 24 bp, Artificial Sequence
  Synthetic oligonucleotide
gttttggatactgcgacattgatg

SEQ ID NO: 372 - DNA, 24 bp, Artificial Sequence
  Synthetic oligonucleotide
catcaatgtcgcagtatccaaaac

SEQ ID NO: 373 - DNA, 22 bp, Artificial Sequence
  Synthetic oligonucleotide
ggggtcctgatagatggcaatt

SEQ ID NO: 374 - DNA, 22 bp, Artificial Sequence
  Synthetic oligonucleotide
tgccatcaatgaggaccccttg

SEQ ID NO: 375 - DNA, 22 bp, Artificial Sequence
  Synthetic oligonucleotide
caaggggtcctcattgatggca

SEQ ID NO: 376 - DNA, 22 bp, Artificial Sequence
  Synthetic oligonucleotide
tgccatcaatcgggaccccttg

SEQ ID NO: 377 - DNA, 22 bp, Artificial Sequence
  Synthetic oligonucleotide
caaggggtcccgattgatggca

SEQ ID NO: 378 - DNA, 20 bp, Artificial Sequence
  Synthetic oligonucleotide
ggaccccttggacaagcaag

SEQ ID NO: 379 - DNA, 20 bp, Artificial Sequence
  Synthetic oligonucleotide
cttgcttgtccaaggggtcc

SEQ ID NO: 380 - DNA, 22 bp, Artificial Sequence
  Synthetic oligonucleotide
cttgggcaagraagggtaccag

SEQ ID NO: 381 - DNA, 22 bp, Artificial Sequence
  Synthetic oligonucleotide
ctggtacccttycttgcccaag

SEQ ID NO: 382 - DNA, 19 bp, Artificial Sequence
  Synthetic oligonucleotide
ggcaagcaaaggtaccagc

SEQ ID NO: 383 - DNA, 19 bp, Artificial Sequence
  Synthetic oligonucleotide
gctggtacctttgcttgcc

SEQ ID NO: 384 - DNA, 19 bp, Artificial Sequence
  Synthetic oligonucleotide
ggcaagcaagygtaccagc

SEQ ID NO: 385 - DNA, 19 bp, Artificial Sequence
  Synthetic oligonucleotide
gctggtacrcttgcttgcc

SEQ ID NO: 386 - DNA, 25 bp, Artificial Sequence
  Synthetic oligonucleotide
tgggcaagcaagagtaccagcttag

SEQ ID NO: 387 - DNA, 25 bp, Artificial Sequence
  Synthetic oligonucleotide
ctaagctggtactcttgcttgccca

SEQ ID NO: 388 - DNA, 21 bp, Artificial Sequence
  Synthetic oligonucleotide
gagacaactttaaagtgtggg

SEQ ID NO: 389 - DNA, 21 bp, Artificial Sequence
  Synthetic oligonucleotide
cccacactttaaagttgtctc

SEQ ID NO: 390 - DNA, 20 bp, Artificial Sequence
  Synthetic oligonucleotide
ctttgaagtgcgggaacgac

SEQ ID NO: 391 - DNA, 20 bp, Artificial Sequence
  Synthetic oligonucleotide
gtcgttcccgcacttcaaag

SEQ ID NO: 392 - DNA, 23 bp, Artificial Sequence
  Synthetic oligonucleotide
gaagtgtgggaccgacctctctc

SEQ ID NO: 393 - DNA, 23 bp, Artificial Sequence
  Synthetic oligonucleotide
gagagaggtcggtcccacacttc

SEQ ID NO: 394 - DNA, 23 bp, Artificial Sequence
  Synthetic oligonucleotide
gaagtgtggaaacgacctctctc

SEQ ID NO: 395 - DNA, 23 bp, Artificial Sequence
  Synthetic oligonucleotide
gagagaggtcgtttccacacttc

SEQ ID NO: 396 - DNA, 22 bp, Artificial Sequence
  Synthetic oligonucleotide
gtgtgggaacaacctctctcag

SEQ ID NO: 397 - DNA, 21 bp, Artificial Sequence
  Synthetic oligonucleotide
ctacgacattcatgcccagac

SEQ ID NO: 398 - DNA, 21 bp, Artificial Sequence
  Synthetic oligonucleotide
gtctgggcatgaatgtcgtag

SEQ ID NO: 399 - DNA, 22 bp, Artificial Sequence
  Synthetic oligonucleotide
gacattgatacccagacctttg

SEQ ID NO: 400 - DNA, 22 bp, Artificial Sequence
  Synthetic oligonucleotide
caaaggtctgggtatcaatgtc

SEQ ID NO: 401 - DNA, 23 bp, Artificial Sequence
  Synthetic oligonucleotide
ctttgctgaccggggagtagatc

SEQ ID NO: 402 - DNA, 23 bp, Artificial Sequence
  Synthetic oligonucleotide
gatctactccccggtcagcaaag

SEQ ID NO: 403 - DNA, 23 bp, Artificial Sequence
  Synthetic oligonucleotide
ctttgctgactgcggagtagatc

SEQ ID NO: 404 - DNA, 23 bp, Artificial Sequence
  Synthetic oligonucleotide
gatctactccgcagtcagcaaag

SEQ ID NO: 405 - DNA, 21 bp, Artificial Sequence
  Synthetic oligonucleotide
gctgactgggtagtagatctg

SEQ ID NO: 406 - DNA, 21 bp, Artificial Sequence
  Synthetic oligonucleotide
cagatctactacccagtcagc

SEQ ID NO: 407 - DNA, 26 bp, Artificial Sequence
  Synthetic oligonucleotide
ctggggagtagttctgctaaaatttg

SEQ ID NO: 408 - DNA, 26 bp, Artificial Sequence
  Synthetic oligonucleotide
caaattttagcagaactactccccag

SEQ ID NO: 409 - DNA, 25 bp, Artificial Sequence
  Synthetic oligonucleotide
gagtagatctgccaaaatttgatgg

SEQ ID NO: 410 - DNA, 25 bp, Artificial Sequence
  Synthetic oligonucleotide
ccatcaaattttggcagatctactc

SEQ ID NO: 411 - DNA, 27 bp, Artificial Sequence
  Synthetic oligonucleotide
gtagatctgctaagatttgatggtttg

SEQ ID NO: 412 - DNA, 27 bp, Artificial Sequence
  Synthetic oligonucleotide
caaaccatcaaatcttagcagatctac

SEQ ID NO: 413 - DNA, 32 bp, Artificial Sequence
  Synthetic oligonucleotide
gtagatctgctaaaatctgatggttgttactg

SEQ ID NO: 414 - DNA, 32 bp, Artificial Sequence
  Synthetic oligonucleotide
cagtaacaaccatcagattttagcagatctac

SEQ ID NO: 415 - DNA, 24 bp, Artificial Sequence
  Synthetic oligonucleotide
gctaaaatttgttggttgttactg

SEQ ID NO: 416 - DNA, 24 bp, Artificial Sequence
  Synthetic oligonucleotide
cagtaacaaccaacaaattttagc

SEQ ID NO: 417 - DNA, 24 bp, Artificial Sequence
  Synthetic oligonucleotide
gctaaaatttcatggttgttactg

SEQ ID NO: 418 - DNA, 24 bp, Artificial Sequence
  Synthetic oligonucleotide
cagtaacaaccatgaaattttagc

SEQ ID NO: 419 - DNA, 27 bp, Artificial Sequence
  Synthetic oligonucleotide
ctaaaatttgatgattgttactgtgac

SEQ ID NO: 420 - DNA, 27 bp, Artificial Sequence
  Synthetic oligonucleotide
gtcacagtaacaatcatcaaattttag

SEQ ID NO: 421 - DNA, 27 bp, Artificial Sequence
  Synthetic oligonucleotide
ctaaaatttgatcgttgttactgtgac

SEQ ID NO: 422 - DNA, 22 bp, Artificial Sequence
  Synthetic oligonucleotide
ctgagagaggttgttcccacac

SEQ ID NO: 423 - DNA, 22 bp, Artificial Sequence
  Synthetic oligonucleotide
gaacgacctccctcaggcttag

SEQ ID NO: 424 - DNA, 22 bp, Artificial Sequence
  Synthetic oligonucleotide
ctaagcctgagggaggtcgttc

SEQ ID NO: 425 - DNA, 21 bp, Artificial Sequence
  Synthetic oligonucleotide
cgacctctcccaggcttagcc

SEQ ID NO: 426 - DNA, 21 bp, Artificial Sequence
  Synthetic oligonucleotide
ggctaagcctgggagaggtcg

SEQ ID NO: 427 - DNA, 23 bp, Artificial Sequence
  Synthetic oligonucleotide
ctcaggcttaccctgggctgtag

SEQ ID NO: 428 - DNA, 23 bp, Artificial Sequence
  Synthetic oligonucleotide
ctacagcccagggtaagcctgag

SEQ ID NO: 429 - DNA, 22 bp, Artificial Sequence
  Synthetic oligonucleotide
cttagcctggcctgtagctatg

SEQ ID NO: 430 - DNA, 22 bp, Artificial Sequence
  Synthetic oligonucleotide
catagctacaggccaggctaag

SEQ ID NO: 431 - DNA, 22 bp, Artificial Sequence
  Synthetic oligonucleotide
ctgggctgtagatatgataaac

SEQ ID NO: 432 - DNA, 22 bp, Artificial Sequence
  Synthetic oligonucleotide
gtttatcatatctacagcccag

SEQ ID NO: 433 - DNA, 22 bp, Artificial Sequence
  Synthetic oligonucleotide
gtagctatgaaaaaccggcagg

SEQ ID NO: 434 - DNA, 22 bp, Artificial Sequence
  Synthetic oligonucleotide
cctgccggtttttcatagctac

SEQ ID NO: 435 - DNA, 21 bp, Artificial Sequence
  Synthetic oligonucleotide
gctatgataaaacggcaggag

SEQ ID NO: 436 - DNA, 21 bp, Artificial Sequence
  Synthetic oligonucleotide
ctcctgccgttttatcatagc

SEQ ID NO: 437 - DNA, 24 bp, Artificial Sequence
  Synthetic oligonucleotide
gctatgataaactggcaggagatt

SEQ ID NO: 438 - DNA, 24 bp, Artificial Sequence
  Synthetic oligonucleotide
aatctcctgccagtttatcatagc

SEQ ID NO: 439 - DNA, 21 bp, Artificial Sequence
  Synthetic oligonucleotide
accggcaggsgattggtggac

SEQ ID NO: 440 - DNA, 21 bp, Artificial Sequence
  Synthetic oligonucleotide
gtccaccaatcscctgccggt

SEQ ID NO: 441 - DNA, 24 bp, Artificial Sequence
  Synthetic oligonucleotide
gataaaccggcagaagattggtgg

SEQ ID NO: 442 - DNA, 24 bp, Artificial Sequence
  Synthetic oligonucleotide
ccaccaatcttctgccggtttatc

SEQ ID NO: 443 - DNA, 29 bp, Artificial Sequence
  Synthetic oligonucleotide
gataaaccggcaggcgattggtggacctc

SEQ ID NO: 444 - DNA, 29 bp, Artificial Sequence
  Synthetic oligonucleotide
gaggtccaccaatcgcctgccggtttatc

SEQ ID NO: 445 - DNA, 23 bp, Artificial Sequence
  Synthetic oligonucleotide
ccggcaggagactggtggacctc

SEQ ID NO: 446 - DNA, 23 bp, Artificial Sequence
  Synthetic oligonucleotide
gaggtccaccagtctcctgccgg

SEQ ID NO: 447 - DNA, 27 bp, Artificial Sequence
  Synthetic oligonucleotide
gtcacagtaacaacgatcaaattttag

SEQ ID NO: 448 - DNA, 30 bp, Artificial Sequence
  Synthetic oligonucleotide
ctaaaatttgatggttwttactgtgacagt

SEQ ID NO: 449 - DNA, 30 bp, Artificial Sequence
  Synthetic oligonucleotide
actgtcacagtaawaaccatcaaattttag

SEQ ID NO: 450 - DNA, 30 bp, Artificial Sequence
  Synthetic oligonucleotide
ctaaaatttgatggttggtactgtgacagt

SEQ ID NO: 451 - DNA, 30 bp, Artificial Sequence
  Synthetic oligonucleotide
actgtcacagtaccaaccatcaaattttag

SEQ ID NO: 452 - DNA, 30 bp, Artificial Sequence
  Synthetic oligonucleotide
ctaaaatttgatggtcgttactgtgacagt

SEQ ID NO: 453 - DNA, 30 bp, Artificial Sequence
  Synthetic oligonucleotide
actgtcacagtaacgaccatcaaattttag

SEQ ID NO: 454 - DNA, 30 bp, Artificial Sequence
  Synthetic oligonucleotide
ctaaaatttgatggtggttactgtgacagt

SEQ ID NO: 455 - DNA, 30 bp, Artificial Sequence
  Synthetic oligonucleotide
actgtcacagtaaccaccatcaaattttag

SEQ ID NO: 456 - DNA, 21 bp, Artificial Sequence
  Synthetic oligonucleotide
ttggcagatgattataagcac

SEQ ID NO: 457 - DNA, 21 bp, Artificial Sequence
  Synthetic oligonucleotide
gtgcttataatcatctgccaa

SEQ ID NO: 458 - DNA, 21 bp, Artificial Sequence
  Synthetic oligonucleotide
ttggcagatagttataagcac

SEQ ID NO: 459 - DNA, 21 bp, Artificial Sequence
  Synthetic oligonucleotide
gtgcttataactatctgccaa

SEQ ID NO: 460 - DNA, 25 bp, Artificial Sequence
  Synthetic oligonucleotide
gttataagcacacgtccttggccct

SEQ ID NO: 461 - DNA, 25 bp, Artificial Sequence
  Synthetic oligonucleotide
agggccaaggacgtgtgcttataac

SEQ ID NO: 462 - DNA, 23 bp, Artificial Sequence
  Synthetic oligonucleotide
gttataagcacgtgtccttggcc

SEQ ID NO: 463 - DNA, 23 bp, Artificial Sequence
  Synthetic oligonucleotide
ggccaaggacacgtgcttataac

SEQ ID NO: 464 - DNA, 23 bp, Artificial Sequence
  Synthetic oligonucleotide
gtccttggcccagaataggactg

SEQ ID NO: 465 - DNA, 23 bp, Artificial Sequence
  Synthetic oligonucleotide
cagtcctattctgggccaaggac

SEQ ID NO: 466 - DNA, 23 bp, Artificial Sequence
  Synthetic oligonucleotide
gtccttggccccgaataggactg

SEQ ID NO: 467 - DNA, 23 bp, Artificial Sequence
  Synthetic oligonucleotide
cagtcctattcggggccaaggac

SEQ ID NO: 468 - DNA, 21 bp, Artificial Sequence
  Synthetic oligonucleotide
ctgaataggattggcagaagc

SEQ ID NO: 469 - DNA, 21 bp, Artificial Sequence
  Synthetic oligonucleotide
gcttctgccaatcctattcag

SEQ ID NO: 470 - DNA, 21 bp, Artificial Sequence
  Synthetic oligonucleotide
cagaagcattatgtactcctg

SEQ ID NO: 471 - DNA, 21 bp, Artificial Sequence
  Synthetic oligonucleotide
caggagtacataatgcttctg

SEQ ID NO: 472 - DNA, 21 bp, Artificial Sequence
  Synthetic oligonucleotide
caggagattagtggacctcgc

SEQ ID NO: 473 - DNA, 21 bp, Artificial Sequence
  Synthetic oligonucleotide
gcgaggtccactaatctcctg

SEQ ID NO: 474 - DNA, 21 bp, Artificial Sequence
  Synthetic oligonucleotide
ggagattggtagacctcgctc

SEQ ID NO: 475 - DNA, 21 bp, Artificial Sequence
  Synthetic oligonucleotide
gagcgaggtctaccaatctcc

SEQ ID NO: 476 - DNA, 24 bp, Artificial Sequence
  Synthetic oligonucleotide
gattggtggacttcgctcttatac

SEQ ID NO: 477 - DNA, 24 bp, Artificial Sequence
  Synthetic oligonucleotide
gtataagagcgaagtccaccaatc

SEQ ID NO: 478 - DNA, 20 bp, Artificial Sequence
  Synthetic oligonucleotide
ggtggacctcactcttatac

SEQ ID NO: 479 - DNA, 20 bp, Artificial Sequence
  Synthetic oligonucleotide
gtataagagtgaggtccacc

SEQ ID NO: 480 - DNA, 20 bp, Artificial Sequence
  Synthetic oligonucleotide
ggtggaccttgctcttatac

SEQ ID NO: 481 - DNA, 20 bp, Artificial Sequence
  Synthetic oligonucleotide
gtataagagcaaggtccacc

SEQ ID NO: 482 - DNA, 23 bp, Artificial Sequence
  Synthetic oligonucleotide
tgcttccctgcgtaaaggagtgg

SEQ ID NO: 483 - DNA, 23 bp, Artificial Sequence
  Synthetic oligonucleotide
ccactcctttacgcagggaagca

SEQ ID NO: 484 - DNA, 24 bp, Artificial Sequence
  Synthetic oligonucleotide
gtaaaggagtggactgtaatcctg

SEQ ID NO: 485 - DNA, 24 bp, Artificial Sequence
  Synthetic oligonucleotide
caggattacagtccactcctttac

SEQ ID NO: 486 - DNA, 24 bp, Artificial Sequence
  Synthetic oligonucleotide
aaaggagtggcctataatcctgcc

SEQ ID NO: 487 - DNA, 24 bp, Artificial Sequence
  Synthetic oligonucleotide
ggcaggattataggccactccttt

SEQ ID NO: 488 - DNA, 24 bp, Artificial Sequence
  Synthetic oligonucleotide
aaaggagtggcccgtaatcctgcc

SEQ ID NO: 489 - DNA, 24 bp, Artificial Sequence
  Synthetic oligonucleotide
ggcaggattacgggccactccttt

SEQ ID NO: 490 - DNA, 24 bp, Artificial Sequence
  Synthetic oligonucleotide
gtaatcctgcctacttcatcacac

SEQ ID NO: 491 - DNA, 24 bp, Artificial Sequence
  Synthetic oligonucleotide
gtgtgatgaagtaggcaggattac

SEQ ID NO: 492 - DNA, 26 bp, Artificial Sequence
  Synthetic oligonucleotide
ctgcctgcttcaacacacagctcctc

SEQ ID NO: 493 - DNA, 26 bp, Artificial Sequence
  Synthetic oligonucleotide
gaggagctgtgtgttgaagcaggcag

SEQ ID NO: 494 - DNA, 24 bp, Artificial Sequence
  Synthetic oligonucleotide
cctgcttcatcccacagctcctcc

SEQ ID NO: 495 - DNA, 24 bp, Artificial Sequence
  Synthetic oligonucleotide
ggaggagctgtgggatgaagcagg

SEQ ID NO: 496 - DNA, 24 bp, Artificial Sequence
  Synthetic oligonucleotide
cttcatcacaccgctcctccctgt

SEQ ID NO: 497 - DNA, 22 bp, Artificial Sequence
  Synthetic oligonucleotide
cattgtgtacttctgtgagtgg

SEQ ID NO: 498 - DNA, 22 bp, Artificial Sequence
  Synthetic oligonucleotide
ccactcacagaagtacacaatg

SEQ ID NO: 499 - DNA, 23 bp, Artificial Sequence
  Synthetic oligonucleotide
cattgtgtactactgtgagtggc

SEQ ID NO: 500 - DNA, 23 bp, Artificial Sequence
  Synthetic oligonucleotide
gccactcacagtagtacacaatg

SEQ ID NO: 501 - DNA, 22 bp, Artificial Sequence
  Synthetic oligonucleotide
gtgtactcctatgagtggcctc

SEQ ID NO: 502 - DNA, 22 bp, Artificial Sequence
  Synthetic oligonucleotide
gaggccactcataggagtacac

SEQ ID NO: 503 - DNA, 23 bp, Artificial Sequence
  Synthetic oligonucleotide
gtgtactcctgggagtggcctct

SEQ ID NO: 504 - DNA, 23 bp, Artificial Sequence
  Synthetic oligonucleotide
agaggccactcccaggagtacac

SEQ ID NO: 505 - DNA, 22 bp, Artificial Sequence
  Synthetic oligonucleotide
ctgtgagtggactctttatatg

SEQ ID NO: 506 - DNA, 22 bp, Artificial Sequence
  Synthetic oligonucleotide
catataaagagtccactcacag

SEQ ID NO: 507 - DNA, 22 bp, Artificial Sequence
  Synthetic oligonucleotide
ctgtgagtggcktctttatatg

SEQ ID NO: 508 - DNA, 22 bp, Artificial Sequence
  Synthetic oligonucleotide
catataaagamgccactcacag

SEQ ID NO: 509 - DNA, 21 bp, Artificial Sequence
  Synthetic oligonucleotide
tggcctctttctatgtggccc

SEQ ID NO: 510 - DNA, 21 bp, Artificial Sequence
  Synthetic oligonucleotide
gggccacatagaaagaggcca

SEQ ID NO: 511 - DNA, 25 bp, Artificial Sequence
  Synthetic oligonucleotide
agtggcctctttgtatgtggccctt

SEQ ID NO: 512 - DNA, 25 bp, Artificial Sequence
  Synthetic oligonucleotide
aagggccacatacaaagaggccact

SEQ ID NO: 513 - DNA, 35 bp, Artificial Sequence
  Synthetic oligonucleotide
cctttcaaaagcccaatgatacagaaatccgacag

SEQ ID NO: 514 - DNA, 35 bp, Artificial Sequence
  Synthetic oligonucleotide
ctgtcggatttctgtatcattgggcttttgaaagg

SEQ ID NO: 515 - DNA, 28 bp, Artificial Sequence
  Synthetic oligonucleotide
caattatacagaaaaccgacagtactgc

SEQ ID NO: 516 - DNA, 28 bp, Artificial Sequence
  Synthetic oligonucleotide
gcagtactgtcggttttctgtataattg

SEQ ID NO: 517 - DNA, 21 bp, Artificial Sequence
  Synthetic oligonucleotide
cgacagtacsgcaatcactgg

SEQ ID NO: 518 - DNA, 21 bp, Artificial Sequence
  Synthetic oligonucleotide
ccagtgattgcsgtactgtcg

SEQ ID NO: 519 - DNA, 21 bp, Artificial Sequence
  Synthetic oligonucleotide
cgacagtactacaatcactgg

SEQ ID NO: 520 - DNA, 24 bp, Artificial Sequence
  Synthetic oligonucleotide
acagggaggagcggtgtgatgaag

SEQ ID NO: 521 - DNA, 28 bp, Artificial Sequence
  Synthetic oligonucleotide
cacagctcctccgtgtgaaaaggaagct

SEQ ID NO: 522 - DNA, 28 bp, Artificial Sequence
  Synthetic oligonucleotide
agcttccttttcacacggaggagctgtg

SEQ ID NO: 523 - DNA, 23 bp, Artificial Sequence
  Synthetic oligonucleotide
ggaagctagggtactatgaatgg

SEQ ID NO: 524 - DNA, 23 bp, Artificial Sequence
  Synthetic oligonucleotide
ccattcatagtaccctagcttcc

SEQ ID NO: 525 - DNA, 22 bp, Artificial Sequence
  Synthetic oligonucleotide
agggttctataaatggacttca

SEQ ID NO: 526 - DNA, 22 bp, Artificial Sequence
  Synthetic oligonucleotide
tgaagtccatttatagaaccct

SEQ ID NO: 527 - DNA, 22 bp, Artificial Sequence
  Synthetic oligonucleotide
gacttcaaggtcaagaagtcac

SEQ ID NO: 528 - DNA, 22 bp, Artificial Sequence
  Synthetic oligonucleotide
gtgacttcttgaccttgaagtc

SEQ ID NO: 529 - DNA, 21 bp, Artificial Sequence
  Synthetic oligonucleotide
gaagtcacaaaaatcccacag

SEQ ID NO: 530 - DNA, 21 bp, Artificial Sequence
  Synthetic oligonucleotide
ctgtgggatttttgtgacttc

SEQ ID NO: 531 - DNA, 24 bp, Artificial Sequence
  Synthetic oligonucleotide
gtcacataaatdccacaggcactg

SEQ ID NO: 532 - DNA, 24 bp, Artificial Sequence
  Synthetic oligonucleotide
cagtgcctgtgghatttatgtgac

SEQ ID NO: 533 - DNA, 22 bp, Artificial Sequence
  Synthetic oligonucleotide
cacataaatcccaaaggcactg

SEQ ID NO: 534 - DNA, 22 bp, Artificial Sequence
  Synthetic oligonucleotide
cagtgcctttgggatttatgtg

SEQ ID NO: 535 - DNA, 22 bp, Artificial Sequence
  Synthetic oligonucleotide
cacataaatcccgcaggcactg

SEQ ID NO: 536 - DNA, 22 bp, Artificial Sequence
  Synthetic oligonucleotide
cagtgcctgcgggatttatgtg

SEQ ID NO: 537 - DNA, 21 bp, Artificial Sequence
  Synthetic oligonucleotide
atcccacagacactgttttgc

SEQ ID NO: 538 - DNA, 21 bp, Artificial Sequence
  Synthetic oligonucleotide
gcaaaacagtgtctgtgggat

SEQ ID NO: 539 - DNA, 22 bp, Artificial Sequence
  Synthetic oligonucleotide
ggcactgtttcgcttcagctag

SEQ ID NO: 540 - DNA, 22 bp, Artificial Sequence
  Synthetic oligonucleotide
ctagctgaagcgaaacagtgcc

SEQ ID NO: 541 - DNA, 29 bp, Artificial Sequence
  Synthetic oligonucleotide
gcgcttcgcttcctggacatccctggggc

SEQ ID NO: 542 - DNA, 29 bp, Artificial Sequence
  Synthetic oligonucleotide
gccccagggatgtccaggaagcgaagcgc

SEQ ID NO: 543 - DNA, 21 bp, Artificial Sequence
  Synthetic oligonucleotide
ccagtgattgtagtactgtcg

SEQ ID NO: 544 - DNA, 21 bp, Artificial Sequence
  Synthetic oligonucleotide
cagtactgcagtcactggcga

SEQ ID NO: 545 - DNA, 21 bp, Artificial Sequence
  Synthetic oligonucleotide
tcgccagtgactgcagtactg

SEQ ID NO: 546 - DNA, 19 bp, Artificial Sequence
  Synthetic oligonucleotide
cagtactgcgatcactggc

SEQ ID NO: 547 - DNA, 19 bp, Artificial Sequence
  Synthetic oligonucleotide
gccagtgatcgcagtactg

SEQ ID NO: 548 - DNA, 21 bp, Artificial Sequence
  Synthetic oligonucleotide
tactgcaatcgctggcgaaat

SEQ ID NO: 549 - DNA, 21 bp, Artificial Sequence
  Synthetic oligonucleotide
atttcgccagcgattgcagta

SEQ ID NO: 550 - DNA, 17 bp, Artificial Sequence
  Synthetic oligonucleotide
gcaatcaccggcgaaat

SEQ ID NO: 551 - DNA, 17 bp, Artificial Sequence
  Synthetic oligonucleotide
atttcgccggtgattgc

SEQ ID NO: 552 - DNA, 22 bp, Artificial Sequence
  Synthetic oligonucleotide
gcaatcactgtcgaaattttgc

SEQ ID NO: 553 - DNA, 22 bp, Artificial Sequence
  Synthetic oligonucleotide
gcaaaatttcgacagtgattgc

SEQ ID NO: 554 - DNA, 26 bp, Artificial Sequence
  Synthetic oligonucleotide
gcaatcactggcaaaattttgctgac

SEQ ID NO: 555 - DNA, 31 bp, Artificial Sequence
  Synthetic oligonucleotide
gcagaccctcagcgccagctagctaattatg

SEQ ID NO: 556 - DNA, 31 bp, Artificial Sequence
  Synthetic oligonucleotide
cataattagctagctggcgctgagggtctgc

SEQ ID NO: 557 - DNA, 26 bp, Artificial Sequence
  Synthetic oligonucleotide
gttttggatactacattgatgcccag

SEQ ID NO: 558 - DNA, 26 bp, Artificial Sequence
  Synthetic oligonucleotide
ctgggcatcaatgtagtatccaaaac

SEQ ID NO: 559 - DNA, 24 bp, Artificial Sequence
  Synthetic oligonucleotide
ctcctgtgagtggatgtggccctt

SEQ ID NO: 560 - DNA, 24 bp, Artificial Sequence
  Synthetic oligonucleotide
aagggccacatccactcacaggag

SEQ ID NO: 561 - DNA, 23 bp, Artificial Sequence
  Synthetic oligonucleotide
caggagagaattgatgttgctgg

SEQ ID NO: 562 - DNA, 23 bp, Artificial Sequence
  Synthetic oligonucleotide
ccagcaacatcaattctctcctg

SEQ ID NO: 563 - DNA, 25 bp, Artificial Sequence
  Synthetic oligonucleotide
gataaaccggcagattggtggacct

SEQ ID NO: 564 - DNA, 25 bp, Artificial Sequence
  Synthetic oligonucleotide
aggtccaccaatctgccggtttatc

SEQ ID NO: 565 - DNA, 35 bp, Artificial Sequence
  Synthetic oligonucleotide
gactggacatcttggacatcttttaaccaggagag

SEQ ID NO: 566 - DNA, 35 bp, Artificial Sequence
  Synthetic oligonucleotide
ctctcctggttaaaagatgtccaagatgtccagtc

(19) United States
(12) Patent Application Publication
(10) Pub. No.: US 2025/0082621 A9
(43) Pub. Date: Mar. 13, 2025

(54) Method To Predict Response To Pharmacological Chaperone Treatment Of Diseases

(71) Applicant: AMICUS THERAPEUTICS, INC., CRANBRY, NJ (US)

(72) Inventors: Elfrida Benjamin, Millstone Twp., NJ (US); Hung V. Do, New Hope, PA (US); Xiaoyang Wu, Edison, NJ (US); John Flanagan, East Windsor, NJ (US); Brandon Alan Wustman, San Diego, CA (US)

(73) Assignee: AMICUS THERAPEUTICS, INC., CRANBURY, NJ (US)

(21) Appl. No.: 17/079,859

(22) Filed: Oct. 26, 2020

Related U.S. Application Data
(62) Division of application No. 16/046,247, filed on Jul. 26, 2018, now Pat. No. 10,813,921.
Application No. 16/046,247 is a continuation of application No. 15/268,662, filed on Sep. 19, 2016, now abandoned.
Application No. 15/268,662 is a continuation of application No. 14/731,603, filed on Jun. 5, 2015, now Pat. No. 9,545,397.
Application No. 14/731,603 is a division of application No. 14/054,369, filed on Oct. 15, 2013, now Pat. No. 9,095,584.
Application No. 14/054,369 is a continuation of application No. 12/855,468, filed on Aug. 12, 2010, now Pat. No. 8,592,362.
Application No. 12/855,468 is a continuation of application No. PCT/US2009/033963, filed on Feb. 12, 2009.
(60) Provisional application No. 61/028,141, filed on Feb. 12, 2008.
(60) Provisional application No. 61/035,684, filed on Mar. 11, 2008.
(60) Provisional application No. 61/093,631, filed on Sep. 2, 2008.
(60) Provisional application No. 61/113,496, filed on Nov. 11, 2008.

(51) Int. Cl. A61K 31/445; C07K 14/00; C12Q 1/34; G01N 33/68

(52) U.S. Cl. CPC A61K 31/445; C07K 14/00; C12Q 1/34; G01N 33/6893; G01N 2333/47; G01N 2333/94; G01N 2800/04; G01N 2800/38; G01N 2800/52

(57) ABSTRACT

The present invention provides methods to determine whether a patient with a lysosomal storage disorder will benefit from treatment with a specific pharmacological chaperone. The present invention exemplifies an in vitro method for determining α-galactosidase A responsiveness to a pharmacological chaperone such as 1-deoxygalactonojirimycin in a cell line expressing a mutant from of α-galactosidase A. The invention also provides a method for diagnosing Fabry disease in patients suspected of having Fabry disease.

### CROSS-REFERENCE TO RELATED APPLICATION

The present application is a continuation of U.S. patent application Ser. No. 14/731,603 filed Jun. 5, 2015, allowed, which is a continuation of U.S. patent application Ser. No. 14/054,369, filed Oct. 15, 2013, now U.S. Pat. No. 9,905,584, which is a divisional of U.S. patent application Ser. No. 12/855,468, filed Aug. 12, 2010, now U.S. Pat. No. 8,592,362, which claims the benefit of International Application No. PCT/US09/033963, filed Feb. 12, 2009, U.S. Provisional Patent Application No. 61/028,141 filed Feb. 12, 2008, U.S. Provisional Patent Application No. 61/035,684, filed Mar. 11, 2008, U.S. Provisional Patent Application No. 61/093,631, filed Sep. 2, 2008 and U.S. Provisional Patent Application No. 61/113,496, filed Nov. 11, 2008. Each of these applications are hereby incorporated by reference and their entirety.

### FIELD OF THE INVENTION

The present invention provides methods to determine whether a patient with a lysosomal storage disorder will benefit from treatment with a specific pharmacological chaperone. The present invention also provides an in vitro method for determining enzyme (e.g., α-galactosidase A, α-glucosidase or glucocerebrosidase) responsiveness to a pharmacological chaperone (e.g., 1-deoxygalactonojirimycin, 1-deoxynojirimycin or isofagomine) in a cell line expressing a mutant form of the enzyme. The invention also provides a method for diagnosing a lysosomal storage disorder (e.g., Fabry disease, Pompe disease or Gaucher disease) in patients suspected of having a lysosomal storage disorder, and implementing the proper treatment based on the diagnosis (e.g., choosing a particular therapeutic agent to administer to the patient).

### BACKGROUND

In the human body, proteins are involved in almost every aspect of cellular function. Proteins are linear strings of amino acids that fold and twist into specific three-dimensional shapes in order to function properly. Certain human diseases result from mutations that cause changes in the amino acid sequence of a protein which reduce its stability and may prevent it from folding properly. The majority of genetic mutations that lead to the production of less stable or misfolded proteins are called missense mutations. These mutations result in the substitution of a single amino acid for another in the protein. Because of this error, missense mutations often result in proteins that have a reduced level of biological activity. In addition to missense mutations, there are also other types of mutations that can result in proteins with reduced biological activity.

Proteins generally fold in a specific region of the cell known as the endoplasmic reticulum, or ER. The cell has quality control mechanisms that ensure that proteins are folded into their correct three-dimensional shape before they can move from the ER to the appropriate destination in the cell, a process generally referred to as protein trafficking. Misfolded proteins are often eliminated by the quality control mechanisms after initially being retained in the ER. In certain instances, misfolded proteins can accumulate in the ER before being eliminated.

The retention of misfolded proteins in the ER interrupts their proper trafficking, and the resulting reduced biological activity can lead to impaired cellular function and ultimately to disease. In addition, the accumulation of misfolded proteins in the ER may lead to various types of stress on cells, which may also contribute to cellular dysfunction and disease.

Lysosomal storage diseases (LSDs) are characterized by deficiencies of lysosomal enzymes due to mutations in the genes encoding the lysosomal enzymes. This results in the pathologic accumulation of substrates of those enzymes, which include lipids, carbohydrates, and polysaccharides. There are about fifty known LSDs to date, which include Gaucher disease, Fabry disease, Pompe disease, Tay Sachs disease and the mucopolysaccharidoses (MPS). Most LSDs are inherited as an autosomal recessive trait, although males with Fabry disease and MPS II are hemizygotes because the disease genes are encoded on the X chromosome. For most LSDs, there is no available treatment beyond symptomatic management. For several LSDs, including Gaucher, Fabry, Pompe, and MPS I and VI, enzyme replacement therapy (ERT) using recombinant enzymes is available. For Gaucher disease, substrate reduction therapy (SRT) also is available in limited situations. SRT employs a small molecule inhibitor of an enzyme required for the synthesis of glucosylceramide (the GD substrate). The goal of SRT is to reduce production of the substrate and reduce pathologic accumulation.

Although there are many different mutant genotypes associated with each LSD, some of the mutations, including some of the most prevalent mutations, are missense mutations which can lead to the production of a less stable enzyme. These less stable enzymes are sometimes prematurely degraded by the ER-associated degradation pathway. This results in the enzyme deficiency in the lysosome, and the pathologic accumulation of substrate. Such mutant enzymes are sometimes referred to in the pertinent art as “folding mutants” or “conformational mutants.”

### Diagnosis of Fabry Disease

Because Fabry disease is rare, involves multiple organs, has a wide age range of onset, and is heterogeneous, proper diagnosis is a challenge. Awareness is low among health care professionals and misdiagnoses are frequent. Some examples of diagnoses seriously considered in patients who were eventually diagnosed with Fabry's disease include: mitral valve prolapse, glomerulonephritis, idiopathic proteinuria, systemic lupus erythematosus, Whipple's disease, acute abdomen, ulcerative colitis, acute intermittent porphyrias, and occult malignancies. Thus, even for classically affected males, diagnosis typically takes from about 5-7 years or even longer. This is a concern because the longer a person has Fabry disease, the more damage is likely to occur in the affected organs and tissues and the more serious the person's condition may become. Diagnosis of Fabry disease is most often confirmed on the basis of decreased α-Gal A activity in plasma or peripheral leukocytes (WBCs) once a patient is symptomatic, coupled with mutational analysis. In females, diagnosis is even more challenging since the enzymatic identification of carrier females is less reliable due to random X-chromosomal inactivation in some cells of carriers. For example, some obligate carriers (daughters of classically affected males) have α-Gal A enzyme activities ranging from normal to very low activities. Since carriers can have normal α-Gal A enzyme activity in leukocytes, only the identification of an α-Gal A mutation by genetic testing provides precise carrier identification and/or diagnosis.

### Treatment of Fabry Disease

One approved therapy for treating Fabry disease diseases is enzyme replacement therapy, which typically involves intravenous, infusion of a purified form of the corresponding wild-type protein (Fabrazyme®, Genzyme Corp.). One of the main complications with protein replacement therapy is attainment and maintenance of therapeutically effective amounts of protein in vivo due to rapid degradation of the infused protein. The current approach to overcome this problem is to perform numerous costly high dose infusions.

Protein replacement therapy has several additional caveats, such as difficulties with large-scale generation, purification, and storage of properly folded protein; obtaining glycosylated native protein; generation of an anti-protein immune response; and inability of protein to cross the blood-brain barrier to mitigate central nervous system pathologies (i.e., low bioavailability). In addition, replacement enzyme cannot penetrate the heart or kidney in sufficient amounts to reduce substrate accumulation in the renal podocytes or cardiac myocytes, which figure prominently in Fabry pathology.

Gene therapy using recombinant vectors containing nucleic acid sequences that encode a functional protein, or using genetically modified human cells that express a functional protein, is also being developed to treat protein deficiencies and other disorders that benefit from protein replacement.

A third, relatively recent approach to treating some enzyme deficiencies involves the use of small molecule inhibitors to reduce production of the natural substrate of deficient enzyme proteins, thereby ameliorating the pathology. This “substrate reduction” approach has been specifically described for a class of about 40 related enzyme disorders called lysosomal storage disorders that include glycosphingolipid storage disorders. The small molecule inhibitors proposed for use as therapy are specific for inhibiting the enzymes involved in synthesis of glycolipids, reducing the amount of cellular glycolipid that needs to be broken down by the deficient enzyme.

It has previously been shown that the binding of small molecule inhibitors of enzymes associated with LSDs can increase the stability of both mutant enzyme and the corresponding wild-type enzyme (see U.S. Pat. Nos. 6,274,597; 6,583,158; 6,589,964; 6,599,919; 6,916,829, and 7,141,582 all incorporated herein by reference). In particular, it was discovered that administration of small molecule derivatives of glucose and galactose, which are specific, selective competitive inhibitors for several target lysosomal enzymes, effectively increased the stability of the enzymes in cells in vitro and, thus, increased trafficking of the enzymes to the lysosome. Thus, by increasing the amount of enzyme in the lysosome, hydrolysis of the enzyme substrates is expected to increase. The original theory behind this strategy was as follows: since the mutant enzyme protein is unstable in the ER (Ishii et al., Biochem. Biophys. Res. Comm. 1996; 220:812-815), the enzyme protein is retarded in the normal transport pathway (ER→Golgi apparatus→endosomes→lysosome) and prematurely degraded. Therefore, a compound which binds to and increases the stability of a mutant enzyme, may serve as a “chaperone” for the enzyme and increase the amount that can exit the ER and move to the lysosomes. In addition, because the folding and trafficking of some wild-type proteins is incomplete, with up to 70% of some wild-type proteins being degraded in some instances prior to reaching their final cellular location, the chaperones can be used to stabilize wild-type enzymes and increase the amount of enzyme which can exit the ER and be trafficked to lysosomes. This strategy has been shown to increase several lysosomal enzymes in vitro and in vivo, including β-glucocerebrosidase and α-glucosidase, deficiencies of which are associated with Gaucher and Pompe disease, respectively.

However, as indicated above, successful candidates for SPC therapy should have a mutation which results in the production of an enzyme that has the potential to be stabilized and folded into a conformation that permits trafficking out of the ER. Mutations which severely truncate the enzyme, such as nonsense mutations, or mutations in the catalytic domain which prevent binding of the chaperone, will not be as likely to be “rescuable” or “enhanceable” using SPC therapy, i.e., to respond to SPC therapy. While missense mutations outside the catalytic site are more likely to be rescuable using SPCs, there is no guarantee, necessitating screening for responsive mutations. This means that, even when Fabry disease is diagnosed by detecting deficient α-Gal A activity in WBCs, it is very difficult, if not impossible, to predict whether a particular Fabry patient will respond to treatment with an SPC without benefit of the present invention. Moreover, since WBCs only survive for a short period of time in culture (in vitro), screening for SPC enhancement of α-Gal A is difficult and not optimal for the patient.

In order to apply SPC therapy effectively, a broadly applicable, fast and efficient method for screening patients for responsiveness to SPC therapy needs to be adopted prior to initiation of treatment. Treatment can then be implemented based on the results of the screening. Thus, there remains in the art a need for relatively non-invasive methods to rapidly assess enzyme enhancement with potential therapies prior to making treatment decisions, for both cost and emotional benefits to the patient.

### SUMMARY OF THE INVENTION

One embodiment of the present invention provides a method for determining whether a patient will be a candidate for SPC therapy. Specifically, the present invention provides an in vitro assay to evaluate protein activity in the presence or absence of an SPC, wherein an SPC that increases the activity of the protein in the in vitro assay is an SPC that can be used for SPC therapy. In one embodiment, the in vitro assay comprises expressing a mutant protein in a host cell, contacting the mutant protein with a candidate SPC, and determining if the mutant protein contacted with the SPC exhibits an increased level of activity (preferably a statistically significant increase) when compared to a mutant protein expressed in a host cell that is not contacted with the candidate SPC. When a candidate SPC increases the activity of a mutant protein according to the assay of the invention, such a candidate SPC can be used for SPC therapy to treat a patient expressing the same mutant protein tested in the in vitro assay.

In one embodiment, the protein is an enzyme. In another embodiment, the protein is a lysosomal enzyme. In yet another embodiment, the protein is α-galactosidase A (α-GAL; α-GAL A). In other embodiments, the protein is alpha-glucosidase (Acid α-glucosidase; α-glucosidase; GAA). In other embodiments, the protein is glucocerebrosidase (β-glucosidase; Gba; GCase).

The present invention also includes the basis for evaluation of SPC as a treatment option for any number of other protein abnormalities and/or enzyme deficiencies and/or a protein folding disorders.

The present invention further provides a written record (e.g., a “treatment reference table”) listing protein mutations and the responsiveness of each of the mutations to SPC therapy. Such a list can be used in determining treatment options for a patient, whereby the patient, or the patient's physician or doctor, can select the proper therapeutic approach, for example, an SPC for treatment by identifying the patient's protein mutation, and cross-referencing the mutation with the list to identify whether an SPC will increase the activity of the patients particular mutant enzyme.

In another embodiment, the “treatment reference table” lists mutations for a lysosomal enzyme, and the treatment reference table is employed to determine the best therapeutic approach to treat a lysosomal storage disorder. In a further embodiment of the invention, the protein is α-Gal A, and the disease is Fabry disease. In other embodiments of the invention, the protein is GAA, and the disease is Pompe disease. In other embodiments of the invention, the protein is Gba, and the disease is Gaucher disease.

In one embodiment, the treatment reference table describes mutant forms of enzyme, such as a lysomal enzyme (e.g., α-Gal A, Gcase, and GAA) and treatment options are ascertained for lysosomal storage disorders (e.g., Fabry, Gacher and Pompe Disease).

In one embodiment, the invention also provides for methods of creating a treatment reference table, wherein the treatment reference table can be for any protein folding disorder or disorder treatable with an SPC. This class of disease includes the other lysosomal storage disorders, Cystic Fibrosis (CFTR) (respiratory or sweat gland epithelial cells), familial hypercholesterolemia (LDL receptor; LPL-adipocytes or vascular endothelial cells), cancer (p53; PTEN-tumor cells), and amyloidoses (transthyretin) among others.

In another embodiment, the present invention provides for methods of treating a patient diagnosed as expressing certain mutant proteins (e.g., lysosomal enzymes such as α-GAL A), wherein activity of the mutant protein (e.g., α-Gal A), when expressed in a host cell, can be increased upon administration of an SPC for that protein (for example, 1-deoxygalactonojirimycin, DGJ, as an SPC for mutant α-GAL A).

The present invention also provides for diagnostic kits containing the components required to perform the assay.

### BRIEF DESCRIPTION OF THE DRAWINGS

FIG. 1A-C. Shows a listing of Fabry mutations generated by site-directed mutagenesis. The text indicates whether HEK-293 cells expressing each of the listed mutations responds to DGJ treatment in the transient transfection assay: italics=not yet tested; bold and underscored=no response to DGJ; plain text (not italicized, bold, or underscored)=response to DGJ.

FIG. 2A-C Shows the responsiveness of different α-Gal A mutations to DGJ treatment. The magnitude of increase in α-Gal A activity levels after DGJ treatment and EC50 values are listed for every tested mutation in FIG. 1A-D that responded to DGJ treatment. The increase in enzyme activity is shown as a percentage of wild type α-Gal A activity.

FIG. 3 Shows representative examples of wild type and mutant α-Gal A responses to DGJ treatment. α-Gal A activity (expressed as nmol/mg protein/hr of 4-MU released) was measured in lysates prepared from transfected HEK 293 cells incubated with increasing concentrations of DGJ. A typical concentration-dependent response is shown for L300P and a typical negative response to DGJ is shown for R227Q. Wild type exhibits high baseline activity and thus does not respond to DGJ in this assay.

FIG. 4 Shows that the mutation response in HEK 293 cells are comparable to patient-derived T-cells, lymphoblasts or white blood cells in vivo. α-Gal A levels measured in three different assays, reported as percentage of wild type, are compared for each mutation examined. α-Gal A levels were measured in T-Cells, lymphoblasts, white blood cells and HEK 293 cells expressing mutant α-Gal A before and after exposure to DGJ. Blank bars indicate basal level (without DGJ treatment) and filled bars indicate the elevated level after DGJ treatment.

FIG. 5 Shows that DGJ-responsive α-Gal A mutations are widely distributed on the α-Gal A protein sequence. Tested Fabry mutations are illustrated on the α-Gal A secondary structure. No significant correlation between response and location on the protein sequence of a mutation was observed, suggesting that responsive as well as non-responsive mutations are distributed widely across the entire protein. Text color indicates DGJ response: green=response; red=no response; brown indicates that of the multiple mutations on that same site some responded to DGJ treatment, while others did not.

FIG. 6 Shows the oligonucleotide primer pairs used to generate the point mutations in the α-Gal A gene through site-directed mutagenesis.

FIG. 7 Shows the α-Gal A cDNA sequence that was mutated through the site-directed mutagenesis.

FIG. 8 Shows the effect of isofagomine tartrate on patient-derived macrophages and lymphoblasts isolated from Gaucher disease patients with different mutations in their glucocerebrosidase (Gba; GCase) enzyme.

FIG. 9 Shows the effect on GL-3 levels of eight-week old male hR301Q α-Gal A Tg/KO mice which were treated for 4 weeks with 300 mg/kg DGJ in drinking water either daily or less frequently (4 days ON/3 days OFF).

FIG. 10 Shows a listing of Pompe mutations generated by site-directed mutagenesis. The text indicates whether COS-7 cells expressing each of the listed mutations responds to DNJ treatment in the transient transfection assay.

FIGS. 11A and 11B Show the nucleic acid sequence of human lysosomal alpha-glucosidase (GAA) (GenBank Accession No.: Y00839).

FIG. 12 Shows the responsiveness of four different GAA mutations to DNJ treatment at concentrations of 0 μM, 20 μM, 50 μM and 100 μM. The increase in enzyme activity is shown as specific activity (nmol/mg protein/hour). FIG. 12 also shows that DNJ promoted processing of GAA to the 95/76/70 kDa forms.

FIG. 13 Shows the responsiveness of Pompe patient-derived fibroblasts to DNJ treatment. The fibroblasts were homozygous for either the P545L or R854X GAA mutation.

FIG. 14 Shows the EC₅₀ for DNJ induced GAA activity in HEK-293 cells transiently transfected with the P545L GAA mutation.

FIG. 15 Shows the responsiveness of Pompe patient-derived lymphocytes to DNJ treatment. The lymphocytes were heterozygous for the (IVS1AS, T>G, -13) GAA splicing defect and a GAA frameshift mutation.

FIGS. 16A, 16B, and 16C Show the amino acid sequence encoded by a human lysosomal alpha-glucosidase (GAA) nucleic acid (GenBank Accession No.: Y00839).

### DETAILED DESCRIPTION

The present invention provides an in vitro assay to provide accurate determination of whether an SPC enhances activity of a mutant protein.

In one embodiment, the protein is a lysosomal enzyme, wherein the lysosomal enzyme, when mutated, causes a lysosomal storage disorder. The concepts of the present invention, however, can be globally applied to any disease or condition characterized by mutant proteins amenable to SPC-therapy, in which the proteins have one or more specific mutations that can be generated in vitro, for example, by site-directed mutagenesis.

In one specific embodiment, the invention provides methods for determining whether an SPC enhances enzyme activity of a mutant α-Gal A enzyme, and can therefore be utilized as an effective therapeutic treatment for a Fabry disease patient expressing the same α-Gal A mutation.

In another specific embodiment, the invention provides methods for determining whether an SPC enhances enzyme activity of a mutant GAA enzyme, and can therefore be utilized as an effective therapeutic treatment for a Pompe disease patient expressing the same GAA mutation.

In another specific embodiment, the invention provides methods for determining whether an SPC enhances enzyme activity of a mutant Gba enzyme, and can therefore be utilized as an effective therapeutic treatment for a Gaucher disease patient expressing the same Gba mutation.

According to the methods of the present invention, assays are provided that allow for the determination of whether a patient expressing a mutant lysosomal enzyme will be a candidate for SPC therapy. The new in vitro assay is extremely sensitive and can be performed on a host cell transfected with a nucleic acid construct encoding a mutant lysosomal enzyme. Specific candidate SPCs can then be assayed to determine if the candidate SPC is capable of increasing the activity of the mutant enzyme expressed by the host cell. Thus, unlike assays which utilize cells derived from a patient with a lysosomal storage disorder, the assay of the invention avoids time consuming steps such as collection of a sample from a patient, purification of cells from the sample, and culturing the cells from the sample in vitro.

The present invention also provides for a method of determining whether a patient expressing a mutant protein (e.g. a lysosomal enzyme) will be a candidate for SPC therapy, wherein a person, for example, a patient's physician or doctor, can look up the mutant protein (e.g. a lysosomal enzyme mutation) in a treatment reference table to determine if the patient's mutation will respond to SPC therapy. The reference table is generated from the results of in vitro analysis of SPC response in a cell line that has been transformed with a nucleic acid vector which encodes the mutant protein.

Furthermore, the invention also provides a “Treatment Reference Table” that provides information describing if a particular SPC will be a successful therapy for enhancing the activity of a specific lysosomal enzyme mutation. According to the present invention, the treatment reference table provides information indicating if a candidate SPC can increase the activity of a mutant lysosomal enzyme expressed by a host cell. Based on the response of different mutations to different SPC therapies, the present invention can provide SPC therapy tailored to the patient's specific mutation.

In one non-limiting embodiment, the mutant protein is a mutant lysosomal enzyme, such as, for example, a mutant α-Gal A, GAA or Gba, and the cell line is transfected with a nucleic acid vector which encodes the mutant lysosomal enzyme.

In another non-limiting embodiment, the present invention provides a method of treating a Fabry patient that includes the step of administering to the Fabry patient a therapeutically effective dose of 1-deoxygalactonojirimycin (DGJ), wherein the patient expresses a mutant α-Gal A, the activity of which, when expressed in a host cell, can be increased when contacted with an SPC (e.g. DGJ). Such α-Gal A mutations treatable according to this method include, but are not limited to A121T, A156V, A20P, A288D, A288P, A292P A348P, A73V, C52R, C94Y, D234E, D244H, D244N, D264Y, E338K, E341D, E358K, E398K, E48K, E59K, E66Q, F113L, G144V, G183D, G260A, G271S, G325D, G328A, G35R, G373D, G373S, H225R, 1219N, I242N, I270T, I289F, I303N, I317T, I354K, I91T, L14P, L166V, L243F, L300F, L310F, L32P, L45R, M267I, M284T, M296I, M296V, M72V, M76R, N224S, N263S, N298K, N298S, N320I, N320Y, N34K, P205R, P259L, P265L, P265R, P293A, P293S, P409S, P40L, P40S, Q279E, Q279H, Q279R, Q280H, Q280K, Q312H, Q321E, Q321R, Q327E, R301P, R342Q, R363C, R363H, R49G, R49L, R49S, S201Y, S276N, S297C, S345P, T194I, V269M, V316E, W340R, W47L, and W95S mutations.

In one embodiment, the following α-Gal A mutations are excluded from the methods of treating a Fabry patient with a therapeutically effective dose of DGJ: D244N, E358K, E59K, E66Q, G183D, G325D, I289F, I91T, L45R, M296V, N263S, N320Y, P205R, P40S, Q279E, R342Q, R363C, R49L, V316E.

One advantage of the assay described by the present invention is its applicability to female patients with an X-linked lysosomal storage disorder, such as Fabry disease. Because of X-chromosome inactivation, a sample taken from a female patient will comprise both normal healthy cells and enzyme deficient mutant cells. An assay for an SPC's effect on such a sample will show an enhancement in enzyme activity due to the normal wild type enzyme expression of the healthy cells even though the diseased cells with the mutant enzyme may not be responsive to the SPC. The present invention overcomes this obstacle because a cell line transfected with a vector encoding a mutant protein will only express the mutant form of the protein, and thus, there will be no wild type protein expressed by the cell line to cause such pseudo enhancement observed in assays with patient derived cells.

In another non-limiting embodiment, the present invention provides a method of treating a Pompe patient that includes the step of administering to the Pompe patient a therapeutically effective dose of 1-deoxynojirimycin (DNJ), wherein the patient expresses a mutant GAA, the activity of which, when expressed in a host cell, can be increased when contacted with an SPC (e.g. DNJ). Such GAA mutations treatable according to this method include, but are not limited to, E262K, P266S, P285R, P285S, L291F, L291H, L291P, M318K, G377R, A445P, Y455C, Y455F, P457L, G483R, G483V, M519V, S529V, P545L, G549R, L552P, Y575S, E579K, A610V, H612Q, A644P, and .DELTA.N470 mutations.

In another non-limiting embodiment, the present invention provides a method of treating a Gaucher patient with a therapeutically effective dose of isofagomine (IFG), wherein the patient expresses a mutant Gba, the activity of which, when expressed in a host cell, can be increased when contacted with an SPC (e.g. IFG).

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Fabry disease” refers to an X-linked inborn error of glycosphingolipid catabolism due to deficient lysosomal α-galactosidase A activity. This defect causes accumulation of globotriaosylceramide (ceramide trihexoside) and related glycosphingolipids in vascular endothelial lysosomes of the heart, kidneys, skin, and other tissues.

The term “atypical Fabry disease” refers to patients with primarily cardiac manifestations of the α-Gal A deficiency, namely progressive globotriaosylceramide (GL-3) accumulation in myocardial cells that leads to significant enlargement of the heart, particularly the left ventricle.

A “carrier” is a female who has one X chromosome with a defective α-Gal A gene and one X chromosome with the normal gene and in whom X chromosome inactivation of the normal allele is present in one or more cell types. A carrier is often diagnosed with Fabry disease.

“Pompe disease” refers to an autosomal recessive LSD characterized by deficient acid alpha glucosidase (GAA) activity which impairs lysosomal glycogen metabolism. The enzyme deficiency leads to lysosomal glycogen accumulation and results in progressive skeletal muscle weakness, reduced cardiac function, respiratory insufficiency, and/or CNS impairment at late stages of disease. Genetic mutations in the GAA gene result in cither lower expression or produce mutant forms of the enzyme with altered stability, and/or biological activity ultimately leading to disease. (see generally Hirschhorn R, 1995, Glycogen Storage Disease Type II: Acid α-Glucosidase (Acid Maltase) Deficiency, The Metabolic and Molecular Bases of Inherited Disease, Scriver et al., eds., McGraw-Hill, New York, 7th ed., pages 2443-2464). The three recognized clinical forms of Pompe disease (infantile, juvenile and adult) are correlated with the level of residual α-glucosidase activity (Reuser A J et al., 1995, Glycogenosis Type II (Acid Maltase Deficiency), Muscle & Nerve Supplement 3, S61-S69). ASSC (also referred to elsewhere as “pharmacological chaperones”) represent a promising new therapeutic approach for the treatment of genetic diseases, such as lysosomal storage disorders (e.g. Pompe Disease).

Infantile Pompe disease (type I or A) is most common and most severe, characterized by failure to thrive, generalized hypotonia, cardiac hypertrophy, and cardiorespiratory failure within the second year of life. Juvenile Pompe disease (type II or B) is intermediate in severity and is characterized by a predominance of muscular symptoms without cardiomegaly. Juvenile Pompe individuals usually die before reaching 20 years of age due to respiratory failure. Adult Pompe disease (type III or C) often presents as a slowly progressive myopathy in the teenage years or as late as the sixth decade (Felice K J et al., 1995, Clinical Variability in Adult-Onset Acid Maltase Deficiency: Report of Affected Sibs and Review of the Literature, Medicine 74, 131-135).

In Pompe, it has been shown that α-glucosidase is extensively modified post-translationally by glycosylation, phosphorylation, and proteolytic processing. Conversion of the 110 kilodalton (kDa) precursor to 76 and 70 kDa mature forms by proteolysis in the lysosome is required for optimum glycogen catalysis.

As used herein, the term “Pompe Disease” refers to all types of Pompe Disease. The formulations and dosing regimens disclosed in this application may be used to treat, for example, Type I, Type II or Type III Pompe Disease.

The term “Gaucher disease” refers to a deficiency of the lysosomal enzyme β-glucocerebrosidase (Gba) that breaks down fatty glucocerebrosides. The fat then accumulates, mostly in the liver, spleen and bone marrow. Gaucher disease can result in pain, fatigue, jaundice, bone damage, anemia and even death. There are three clinical phenotypes of Gaucher disease. Patients with, Type 1 manifest either early in life or in young adulthood, bruise easily and experience fatigue due to anemia, low blood platelets, enlargement of the liver and spleen, weakening of the skeleton, and in some instances have lung and kidney impairment. There are no signs of brain involvement. In Type II, early-onset, liver and spleen enlargement occurs by 3 months of age and there is extensive brain involvement. There is a high mortality rate by age 2. Type III is characterized by liver and spleen enlargement and brain seizures. The β-glucocerebrosidase gene is located on the human 1q21 chromosome. Its protein precursor contains 536 amino acids and its mature protein is 497 amino acids long.

A “patient” refers to a subject who has been diagnosed with or is suspected of having a particular disease. The patient may be human or animal.

A “Fabry disease patient” refers to an individual who has been diagnosed with or suspected of having Fabry disease and has a mutated α-Gal A as defined further below. Characteristic markers of Fabry disease can occur in male hemizygotes and female carriers with the same prevalence, although females typically are less severely affected.

A “Pompe disease patient” refers to an individual who has been diagnosed with or suspected of having Pompe disease and has a mutated GAA as defined further below.

A “Gaucher disease patient” refers to an individual who has been diagnosed with or suspected of having Gaucher disease and has a mutated Gba as defined further below.

Human α-galactosidase A (α-Gal A) refers to an enzyme encoded by the human GLA gene. The human α-Gal A enzyme consists of 429 amino acids and is in GenBank Accession No. U78027.

In one non-limiting embodiment, human lysosomal alpha-glucosidase (Acid α-glucosidase; GAA) is a lysosomal enzyme which hydrolyzes alpha-1,4- and alpha-1,6-linked-D-glucose polymers present in glycogen, maltose, and isomaltose. Alternative names are as follows: glucoamylase; 1,4-α-D-glucan glucohydrolase; amyloglucosidase; gamma-amylase; and exo-1,4-α-glucosidase. The human GAA gene has been mapped to chromosome 17q25.2-25.3 and has nucleotide and amino acid sequences depicted in GenBank Accession No. Y00839.

The term “human Gba gene” refers to the gene encoding acid β-glucosidase, also referred to as glucocerebrosidase or Gba. The Gba gene is on chromosome 1q21 and involves 11 exons (GenBank Accession No. J03059). There is also a homologous pseudogene for Gba located about 16 kb downstream of the Gba gene (GenBank Accession No. M16328).

The “human Gba” protein refers to the wild-type human Gba protein. The Gba protein consists of 536 amino acids and is in GenBank Accession No. J03059.

The term “mutant protein” includes a protein which has a mutation in the gene encoding the protein which results in the inability of the protein to achieve a stable conformation under the conditions normally present in the ER. The failure to achieve a stable conformation results in a substantial amount of the enzyme being degraded, rather than being transported to the lysosome. Such a mutation is sometimes called a “conformational mutant.” Such mutations include, but are not limited to, missense mutations, and in-frame small deletions and insertions.

As used herein in one embodiment, the term “mutant α-Gal A” includes an α-Gal A which has a mutation in the gene encoding α-Gal A which results in the inability of the enzyme to achieve a stable conformation under the conditions normally present in the ER. The failure to achieve a stable conformation results in a substantial amount of the enzyme being degraded, rather than being transported to the lysosome.

Non-limiting, exemplary α-Gal A mutations associated with Fabry disease which result in unstable α-Gal A include L32P; N34S; T41I; M51K; E59K; E66Q; I91T; A97V; R100K; R112C; R112H; F113L; T141L; A143T; G144V; S148N; A156V; L166V; D170V; C172Y; G183D; P205T; Y207C; Y207S; N215S; A228P; S235C; D244N; P259R; N263S; N264A; G272S; S276G; Q279E; Q279K; Q279H; M284T; W287C; I289F; M296I; M296V; L300P; R301Q; V316E; N320Y; G325D; G328A; R342Q; E358A; E358K; R363C; R363H; G370S; and P409A.

As used herein in one embodiment, the term “mutant GAA” includes a GAA which has a mutation in the gene encoding GAA which results in the inability of the enzyme to achieve a stable conformation under the conditions normally present in the ER. The failure to achieve a stable conformation results in a substantial amount of the enzyme being degraded, rather than being transported to the lysosome.

As used herein in one embodiment, the term “mutant Gba” includes a Gba which has a mutation in the gene encoding Gba which results in the inability of the enzyme to achieve a stable conformation under the conditions normally present in the ER. The failure to achieve a stable conformation results in a substantial amount of the enzyme being degraded, rather than being transported to the lysosome.

As used herein, the term “specific pharmacological chaperone” (“SPC”) or “pharmacological chaperone” refers to any molecule including a small molecule, protein, peptide, nucleic acid, carbohydrate, etc. that specifically binds to a protein and has one or more of the following effects: (i) enhances the formation of a stable molecular conformation of the protein; (ii) induces trafficking of the protein from the ER to another cellular location, preferably a native cellular location, i.e., prevents ER-associated degradation of the protein; (iii) prevents aggregation of misfolded proteins; and/or (iv) restores or enhances at least partial wild-type function and/or activity to the protein. A compound that specifically binds to e.g., α-Gal A, GAA or Gba, means that it binds to and exerts a chaperone effect on the enzyme and not a generic group of related or unrelated enzymes. More specifically, this term does not refer to endogenous chaperones, such as BiP, or to non-specific agents which have demonstrated non-specific chaperone activity against various proteins, such as glycerol, DMSO or deuterated water, i.e., chemical chaperones (see Welch et al., Cell Stress and Chaperones 1996; 1(2):109-115; Welch et al., Journal of Bioenergetics and Biomembranes 1997; 29(5):491-502; U.S. Pat. Nos. 5,900,360; 6,270,954; and 6,541,195). In the present invention, the SPC may be a reversible competitive inhibitor.

A “competitive inhibitor” of an enzyme can refer to a compound which structurally resembles the chemical structure and molecular geometry of the enzyme substrate to bind the enzyme in approximately the same location as the substrate. Thus, the inhibitor competes for the same active site as the substrate molecule, thus increasing the Km. Competitive inhibition is usually reversible if sufficient substrate molecules are available to displace the inhibitor, i.e., competitive inhibitors can bind reversibly. Therefore, the amount of enzyme inhibition depends upon the inhibitor concentration, substrate concentration, and the relative affinities of the inhibitor and substrate for the active site.

Following is a description of some specific pharmacological chaperones (SPCs) contemplated by this invention:

In one particular non-limiting embodiment, the SPC is 1-deoxygalactonorjirimycin which refers to a compound having the following structures:

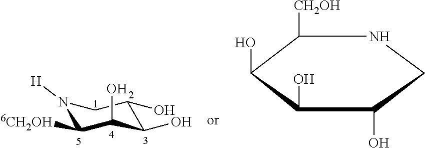

or a pharmaceutically acceptable salt, ester or prodrug of 1-deoxygalactonorjirimycin. The hydrochloride salt of DGJ is known as migalastat hydrochloride (Migalastat).

Still other SPCs for α-Gal A are described in U.S. Pat. Nos. 6,274,597, 6,774,135, and 6,599,919 to Fan et al., and include α-3,4-di-epi-homonojirimycin, 4-epi-fagomine, α-allo-homonojirimycin, N-methyl-deoxygalactonojirimycin, β-1-C-butyl-deoxygalactonojirimycin, α-galacto-homonojirimycin, calystegine A₃, calystegine B₂, calystegine B₃, N-methyl-calystegine A₃, N-methyl-calystegine B₂ and N-methyl-calystegine B₃.

In one particular non-limiting embodiment, the SPC is isofagomine (IFG; (3R,4R,5R)-5-(hydroxymethyl)-3,4-piperidinediol) which is represented by the following formula:

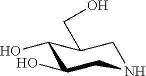

or a pharmaceutically acceptable salt, ester or prodrug of isofagomine, such as, for example, IFG tartrate (see, e.g., U.S. Patent Application Publication 20070281975.) IFG has a molecular formula of C₆H₁₃NO3 and a molecular weight of 147.17. This compound is further described in U.S. Pat. No. 5,844,102 to Sierks et al., and U.S. Pat. No. 5,863,903, to Lundgren et al.

Still other SPCs for Gba are described in U.S. Pat. No. 6,916,829 to Fan et al., and include C-benzyl isofagomine and derivatives, N-alkyl (C9-12)-DNJ, Glucoimidazole (and derivatives), C-alkyl-IFG (and derivatives), N-alkyl-β-valeinamines, Fluphenozine, N-dodecyl-DNJ, calystegines A₃, B₁, B₂ and C₁

In one particular non-limiting embodiment, the SPC is 1-deoxynorjirimycin (1-DNJ), which is represented by the following formula:

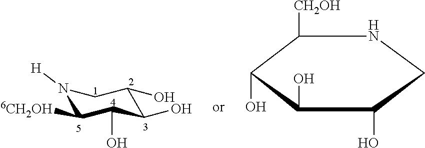

or a pharmaceutically acceptable salt, ester or prodrug of 1-deoxynorjirimycin. In one embodiment, the salt is hydrochloride salt (i.e. 1-deoxynojirimycin-HCl).

Still other SPCs for GAA are described in U.S. Pat. Nos. 6,274,597; 6,583,158; 6,599,919 and 6,916,829 to Fan et al., and U.S. Published Application No. 2006/0264467, and include N-methyl-DNJ, N-ethyl-DNJ, N-propyl-DNJ, N-butyl-DNJ, N-pentyl-DNJ, N-hexyl-DNJ, N-heptyl-DNJ, N-octyl-DNJ, N-nonyl-DNJ, N-methylcyclopropyl-DNJ, N-methylcyclopentyl-DNJ, N-2-hydroxyethyl-DNJ, 5-N-carboxypentyl DNJ, α-homonojirimycin, and castanospermine.

As used herein, the term “specifically binds” refers to the interaction of a pharmacological chaperone with a protein such as α-Gal A, Gba or GAA, specifically, an interaction with amino acid residues of the protein that directly participate in contacting the pharmacological chaperone. A pharmacological chaperone specifically binds a target protein, e.g., α-Gal A, Gba or GAA, to exert a chaperone effect on the protein and not a generic group of related or unrelated proteins. The amino acid residues of a protein that interact with any given pharmacological chaperone may or may not be within the protein's “active site.” Specific binding can be evaluated through routine binding assays or through structural studies, e.g., co-crystallization, NMR, and the like. The active site for α-Gal A, Gba or GAA is the substrate binding site.

“Deficient α-Gal A activity” refers to α-Gal A activity in cells from a patient which is below the normal range as compared (using the same methods) to the activity in normal individuals not having or suspected of having Fabry or any other disease (especially a blood disease).

“Deficient Gba activity” refers to Gba activity in cells from a patient which is below the normal range as compared (using the same methods) to the activity in normal individuals not having or suspected of having Gaucher or any other disease.

“Deficient GAA activity” refers to GAA activity in cells from a patient which is below the normal range as compared (using the same methods) to the activity in normal individuals not having or suspected of having Pompe or any other disease.

As used herein, the terms “enhance α-Gal A activity,” “enhance Gba activity,” and “enhance GAA activity” or “increase α-Gal A activity,” “increase Gba activity,” and “increase GAA activity” refer to increasing the amount of α-Gal A, Gba or GAA, respectively, that adopts a stable conformation in a cell contacted with a pharmacological chaperone specific for the α-Gal A, Gba or GAA, relative to the amount in a cell (preferably of the same cell-type or the same cell, e.g., at an earlier time) not contacted with the pharmacological chaperone specific for the α-Gal A, Gba or GAA. This term also refers to increasing the trafficking of α-Gal A, Gba or GAA to the lysosome in a cell contacted with a pharmacological chaperone specific for the α-Gal A, Gba or GAA, relative to the trafficking of α-Gal A, Gba or GAA not contacted with the pharmacological chaperone specific for the protein. These terms refer to both wild-type and mutant α-Gal A, Gba or GAA. In one embodiment, the increase in the amount of α-Gal A, Gba or GAA in the cell is measured by measuring the hydrolysis of an artificial substrate in lysates from cells that have been treated with the SPC. An increase in hydrolysis is indicative of increased α-Gal A, Gba or GAA activity.

The term “α-Gal A activity” refers to the normal physiological function of a wild-type α-Gal A in a cell. For example, α-Gal A activity includes hydrolysis of GL-3.

The term “Gba activity” refers to the normal physiological function of a wild-type αGba in a cell. For example, Gba activity includes metabolism of fatty glucocerebrosides.

The term “GAA activity” refers to the normal physiological function of a wild-type Gaa in a cell. For example, GAA activity includes lysosomal glycogen metabolism.

A “responder” is an individual diagnosed with or suspected of having a lysosomal storage disorder, such, for example, but not limited to, Fabry disease, Pompe disease or Gaucher disease, whose cells exhibit sufficiently increased α-Gal A, GAA or Gba activity, respectively, and/or amelioration of symptoms or improvement in surrogate markers, in response to contact with an SPC. Non-limiting examples of improvements in surrogate markers for Fabry and Pompe disease are disclosed in U.S. Ser. Nos. 60/909,185 and 61/035,869, respectively.

Non-limiting examples of improvements in surrogate markers for Fabry disease disclosed in U.S. Ser. No. 60/909,185 include increases in α-Gal A levels or activity in cells (e.g., fibroblasts) and tissue; reductions in of GL-3 accumulation; decreased plasma concentrations of homocysteine and vascular cell adhesion molecule-1 (VCAM-1); decreased GL-3 accumulation within myocardial cells and valvular fibrocytes; reduction in cardiac hypertrophy (especially of the left ventricle), amelioration of valvular insufficiency, and arrhythmias; amelioration of proteinuria; decreased urinary concentrations of lipids such as CTH, lactosylceramide, ceramide, and increased urinary concentrations of glucosylceramide and sphingomyelin (Fuller et al., Clinical Chemistry. 2005; 51:688-694); the absence of laminated inclusion bodies (Zebra bodies) in glomerular epithelial cells; improvements in renal function; mitigation of hypohidrosis; the absence of angiokeratomas; and improvements hearing abnormalities such as high frequency sensorineural hearing loss progressive hearing loss, sudden deafness, or tinnitus. Improvements in neurological symptoms include prevention of transient ischemic attack (TIA) or stroke; and amelioration of neuropathic pain manifesting itself as acroparaesthesia (burning or tingling in extremities).

The dose that achieves one or more of the aforementioned responses is a “therapeutically effective dose.”

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an mRNA band on a gel, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acids include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

### Method of Determining Treatment Options

To easily determine whether SPC therapy will be a viable treatment for patients, for example, Fabry, Pompe or Gaucher patients, and including female carriers of X-linked lysosomal storage disorders such as Fabry disease, a simple, non-invasive SPC rescue assay of protein activity in a cell line expressing a mutant form of the protein was developed.

### In Vitro Assay

In one embodiment, the diagnostic method of the present invention involves transforming a cell line with a nucleic acid vector which encodes a mutant lysosomal enzyme, for example, α-Gal A, GAA or Gba. The cell line is then treated with or without an SPC, e.g., DGJ, DNJ or IFG, for a sufficient time period to demonstrate enhancement (i.e., increase) of α-Gal A, GAA or Gba activity. The transformed cells are then lysed, and the lysate is used in an assay to determine enzyme activity. A sufficient increase in α-Gal A, GAA or Gba activity in the lysates from cells treated with the SPC over the activity in the lysates from untreated cells indicates that a patient who expresses α-Gal A, GAA or Gba with the same mutation as the cell line will likely respond to SPC therapy (i.e., the patient will be a “responder”).

### Transient Transfection of a Cell Line and Expression of a Mutant Lysosmal Enzyme

In one embodiment, to identify SPC-responsive mutations, all known lysosomal enzyme (e.g., α-Gal A, GAA or Gba) mutations, for example, missense mutations and in-frame small deletions and insertions, can be generated according to techniques known in the art, for example, by site-directed mutagenesis. Mutant enzyme constructs can then be transiently expressed in a cell line, for example, mammalian COS-7, HEK-293 or GripTite 293 MSR (Invitrogen Corp., Carlsbad, Calif., U.S.A.) cells. Transformed cells can then be incubated with increasing concentrations of SPC and enzymatic activity can be measured in cell lysates.

Mutagenesis: Nucleic acid vectors encoding a mutant protein (e.g. mutant α-Gal A, GAA or Gba) can be generated by conventional molecular biology, microbiology, and recombinant DNA techniques within the skill of the art. Such techniques are explained fully in the literature. (See, e. g., Sambrook, Fritsch & Maniatis, 2001, Molecular Cloning: A Laboratory Manual, Third Edition, Cold Spring Harbor Laboratory Press, Cold Spring Harbor, N.Y.; Glover, ed., 1985, DNA Cloning: A Practical Approach, Volumes I and II, Second Edition; Gait, M. J., ed., 1984, Oligonucleotide Synthesis: A practical approach; Hames, B. D. & Higgins, S. J. eds., 1985, Nucleic Acid Hybridization; Hames, B. D. & Higgins, S. J., eds., 1984, Transcription And Translation; Freshney, R. I., 2000, Culture of Animal Cells: A Manual of Basic Technique; Woodward, J., 1986, Immobilized Cells And Enzymes: A practical approach, IRL Press; Perbal, B. E., 1984, A Practical Guide To Molecular Cloning). For example, a single α-Gal A, GAA or Gba mutation can be introduced into a nucleic acid encoding a wild type α-Gal A, GAA or Gba gene through site directed mutagenesis of a nucleic acid encoding the wild type enzyme.

Transient Transfection and Expression: The coding sequences of the gene to be delivered, for example, a mutant α-Gal A, GAA or Gba, are operably linked to expression control sequences, e.g., a promoter that directs expression of the gene. As used herein, the phrase “operatively linked” refers to the functional relationship of a polynucleotide/gene with regulatory and effector sequences of nucleotides, such as promoters, enhancers, transcriptional and translational stop sites, and other signal sequences. For example, operative linkage of a nucleic acid to a promoter refers to the physical and functional relationship between the polynucleotide and the promoter such that transcription of DNA is initiated from the promoter by an RNA polymerase that specifically recognizes and binds to the promoter. The promoter directs the transcription of RNA from the polynucleotide. Expression of a mutant protein (e.g. mutant α-Gal A, GAA or Gba) may be controlled by any promoter/enhancer element known in the art, but these regulatory elements must be functional in the host selected for expression.

In one specific embodiment, a vector is used in which the coding sequences and any other desired sequences are flanked by regions that promote homologous recombination at a desired site in the genome, thus providing for expression of the construct from a nucleic acid molecule that has integrated into the genome (See Koller and Smithies, 1989, Proc. Natl. Acad. Sci. USA, 86:8932-8935; Zijlstra et al., 1989, Nature 342:435-438; U.S. Pat. No. 6,244,113 to Zarling et al.; and U.S. Pat. No. 6,200,812 to Pati et al.).

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. In one embodiment, a host cells that is transfected with a vector encoding a mutant α-Gal A, GAA or Gba can be used for screening a candidate SPC, for example, DGJ, DNJ or IFG, to determine if the candidate SPC is an effective compound for increasing the activity of the mutant α-Gal A, GAA or Gba expressed by the host cell.

The term “expression system” means a host cell and compatible vector under suitable conditions, e.g., for the expression of a protein coded for by foreign DNA carried by the vector and introduced to the host cell. Expression systems include mammalian host cells and vectors. Suitable cells include PC12 cells, CHO cells, HeLa cells, GripTite 293 MSR cells (Invitrogen Corp., Carlsbad, Calif., U.S.A.), HEK-293 (also known as 293 cells) and 293T cells (derived from human embryonic kidney cells), COS cells (e.g. COS-7 cells), mouse primary myoblasts, NIH 3T3 cells.

Suitable vectors include viruses, such as adenoviruses, adeno-associated virus (AAV), vaccinia, herpesviruses, baculoviruses and retroviruses, parvovirus, lentivirus, bacteriophages, cosmids, plasmids, fungal vectors, naked DNA, DNA lipid complexes, and other recombination vehicles typically used in the art which have been described for expression in a variety of eukaryotic and prokaryotic hosts, and may be used for gene therapy as well as for simple protein expression.

In one non-limiting example, transient transfection can be carried out in GripTite 293 MSR cells (Invitrogen Corp., Carlsbad, Calif., U.S.A.) using the reagent Fugene HD (Roche). The cells can be seeded in a suitable assay container, such as a 96-well plate (Costar) at a density of, for example, 7.5-10 k cells/well, and incubated under suitable conditions, such as, for example, 37° C., 5% CO₂ for 24 hours before transfection. After transfection with expression constructs containing a specific α-Gal A mutant, cells can be incubated again in, for example, 37° C., 5% CO₂ for one hour before adding DGJ at 50 nM to 1 mM. Cells can then be incubated for 4-5 days before lysis and assay.

Enzyme Activity/Enhancement Assay: Typically, following incubation with an SPC (e.g. DGJ, DNJ or IFG), host cells are lysed by the addition of lysis buffer (or deionized water) and physical disruption (pipetting, vortexing and/or agitation, and/or sonication) at room temperature or on ice, followed by pooling of the lysates on ice, then splitting the pooled lysate into small aliquots and freezing.

The lysates can be thawed immediately prior to the assay and should be suspended by use of a vortex mixer and sonicated prior to addition to appropriate wells e.g., in a microplate. In the context of Fabry disease, N-acetylgalactosamine (GalNAc) is then added to each well (to inhibit α-galactosidase B), followed by a short incubation. 4-methylumbelliferyl-α-D-galactopyranoside (4-MU Gal), or other appropriate labeled DGJ substrate, is then added and the plate is gently mixed for a brief period of time, covered, and incubated at 37° C. for a sufficient time for substrate hydrolysis, usually about 1 hour. To stop the reaction, NaOH-glycine buffer, pH 10.7, is added to each well and the plate is read on a fluorescent plate reader (e.g. Wallac 1420 Victor3™ or similar instrument). Excitation and emission wavelengths were customarily set at 355 nm and 460 nm, respectively. One unit of enzyme activity is defined as the amount of enzyme that catalyzes the hydrolysis of 1 nmole of 4-methylumbelliferone per hour. For each patient sample at least three normal samples may be tested concurrently.

Various modifications of this assay will be readily ascertainable to one of ordinary skill in the art. Examples of artificial substrates that can be used to detect α-Gal A activity include but are not limited to p-nitrophenyl-α-D-galactopyranoside and 4-MU GAL. Obviously, only substrates that can be cleaved by human α-Gal A are suitable for use. It is noted that while use of a fluorogenic substrate is preferred, other methods of determining enzymatic activity are contemplated for use in the method, including using chromogenic substrates or immunoquantification techniques.

In one specific example, following incubation with an SPC, for example, DGJ, the host cells can be washed two times with PBS then incubated in 200 μl fresh media at 37° C., 5% CO₂ for two hours followed by 2 additional PBS washes. After, cells can be lysed in 60 μL Lysis Buffer (27 mM sodium citrate/46 mM sodium phosphate dibasic, 0.5% Triton X-100, pH 4.6). Ten μL lysate can then be added to 50 μL assay buffer (Lysis Buffer without Triton X-100, but containing 6 mM 4-MU-α-D-galactopyranoside (4-MUG) and 117 mM N-acetyl-D-galactosamine (GalNac)), and incubated at 37° C. for 1 hr. Seventy L Stop Solution (0.4 M glycine, pH 10.8) can then be added and fluorescence read on a Victor plate reader (Perkin Elmer) at 355 nm excitation and 460 nm emission. Raw fluorescence counts can be background subtracted as defined by counts from substrate solution only. A MicroBCA Protein Assay Kit (Pierce) was used according to manufacturer's instructions to determine protein concentration from 40 μL of cell lysate. A 4-methylumbelliferone (4-MU) standard curve ranging from 30 μM to 1.3 nM was run in parallel for calculation of absolute α-Gal A activity expressed as nmoles/mg protein/hr or further normalized to % of untreated wild type enzyme activity.

### Treatment Reference Table

In another embodiment, the methods described supra can be used to generate a “treatment reference table” or “treatment therapy table,” wherein the treatment reference table comprises a list of protein mutations, and further wherein the table indicates the responsiveness of each mutation to an SPC, such as DGJ, DNJ or IFG. The treatment reference table can then be used to determine if a particular SPC, for example, DGJ, DNJ or IFG, would be an effective SPC for treating a patient with a particular α-Gal A, GAA or Gba mutation, respectively.

As used herein “treatment therapy table” or “treatment reference table” refers to any written record that conveys whether a particular mutation is responsive to SPC therapy, and is not necessarily limited to written records presented in tabular form.

In one embodiment, the treatment reference table can be used by a treating physician or clinical professional to select an SPC for treating a patient, for example, a Fabry, Pompe or Gaucher patient who expresses a specific mutant α-Gal A, GAA or Gba, respectively, wherein the SPC is selected because the treatment reference table identifies the SPC as a compound that can increase the activity of the patient's mutant α-Gal A, GAA or Gba when the mutant α-Gal A, GAA or Gba is expressed in a host cell.

### Treatable Disorders

While the present application has been discussed largely in the context of Fabry, Pompe and Gaucher diseases, and the SPCs DGJ, DNJ and IFG, respectively, it should be understood that it is applicable to any SPC and disease. In one non-limiting embodiment, a treatment reference table can be generated for any candidate SPC and any lysosomal storage disorder, or any disorder involving protein misfolding. These diseases include other lysosomal storage disorders, for example, Cystic Fibrosis (CFTR) (respiratory or sweat gland epithelial cells), familial hypercholesterolemia (LDL receptor; LPL-adipocytes or vascular endothelial cells), cancer (p53; PTEN-tumor cells), Alzheimer's disease (α-secretase), Parkinson's disease (glucocerebrosidase), obesity (MC4R), and amyloidoses (transthyretin) among others.

### Eligibility Determination Criteria

The criteria for determining eligibility for SPC therapy depends on the type of mutant GLA, GAA or Gba a patient expresses. In one embodiment, patients with Fabry, Pompe, or Gaucher disease could be categorized as eligible for SPC therapy if α-Gal A, GAA or Gba activity, respectively, in a host cell expressing the same mutation as the patient, in the presence of an SPC such as DGJ, DNJ or IFG, is at least about 1.5- to 20-fold (2% to 100%) activity of a host cell expressing a wild type α-Gal A, GAA or Gba.

This discovery provides a method for improving the diagnosis of and facilitating clinical treatment decisions for Fabry, Pompe and Gaucher diseases in particular, and lysosomal storage disease in general. Moreover, this method can be extended to a wide range of genetically defined diseases in appropriate cell types. This class of disease includes the other lysosomal storage disorders, Cystic Fibrosis (CFTR) (respiratory or sweat gland epithelial cells), familial hypercholesterolemia (LDL receptor; LPL-adipocytes or vascular endothelial cells), cancer (p53; PTEN-tumor cells), Alzheimer's disease (α-secretase), Parkinson's disease (glucocerebrosidase), obesity (MC4R), and amyloidoses (transthyretin) among others.

### Kits

The present invention also provides for a commercial diagnostic test kit in order to make therapeutic treatment decisions. The kit provides all materials discussed above and more particularly in the Examples below, for preparing and running each assay in one convenient package, optionally including instructions and an analytic guide.

As one non-limiting example, a kit for evaluating α-Gal A activity may contain, at a minimum:
a. a panel of host cells, each expressing a mutant α-Gal A, or alternatively, a host cell, a vector encoding a mutant α-Gal A, and a means of transfecting the host cell such that the host cell expresses the mutant α-Gal A;
b. a specific pharmacological chaperone;
c. a chromogenic or fluorogenic substrate for the enzyme assay (including an appropriate standard); and
d. GalNAc.
   The kit may also contain instructions for optimally performing the protein enhancement assay. In another embodiment, the kit will contain the appropriate tubes, buffers (e.g., lysis buffer), and microplates.

In one embodiment, the SPC is supplied in dry form, and will be re-constituted prior to addition.

Patients who express a mutant α-Gal A, GAA or Gba that previously tested positive for enzyme enhancement with a candidate SPC in assays of the present invention can then be treated with that candidate SPC agent, whereas patients who express a mutant α-Gal A, GAA or Gba that does not display enzyme enhancement with a candidate SPC can avoid treatment which will save money and prevent the emotional toll of not responding to a treatment modality.

### EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### Example 1: Identification of Fabry Disease-Causing Mutations That are Responsive to the Pharmacological Chaperone DGJ

The present Example provides the in vitro diagnostic assay to determine a Fabry patient's responsiveness to a specific pharmacological chaperone.

### Introduction

Fabry disease is a lysosomal storage disorder caused by mutations in the gene that encodes α-galactosidase A (α-GAL A). Over 600 Fabry mutations have been reported, and about 60% are missense. The iminosugar DGJ is currently being studied in Phase 2 clinical trials as a pharmacological chaperone for the treatment of Fabry disease. Previously, it has been shown that DGJ mediates selective and dose-dependent increases in α-Gal A levels in many Fabry patient-derived lymphoid cell lines. To identify additional DGJ-responsive mutations, GripTite 293 MSR, (Invitrogen Corp., Carlsbad, Calif., U.S.A.) cells were transiently transfected with expression vectors containing all known α-Gal A missense mutations and several in-frame small deletions and insertions generated by site-directed mutagenesis. Mutant α-Gal A constructs were transiently expressed in HEK-293 cells. Cells were incubated with increasing concentrations of DGJ and α-Gal A activity was measured in cell lysates. Assay validation has been carried out on more than 35 missense mutations and the results obtained in HEK-293 cells were similar to those obtained from both Fabry patient-derived lymphoid cells and primary T-cell cultures (see U.S. Ser. No. 11/749,512), as well as to the α-Gal A enzyme responses observed in the white blood cells of Fabry patients after oral administration of DGJ in Phase 2 clinical trials.

### Methods and Materials

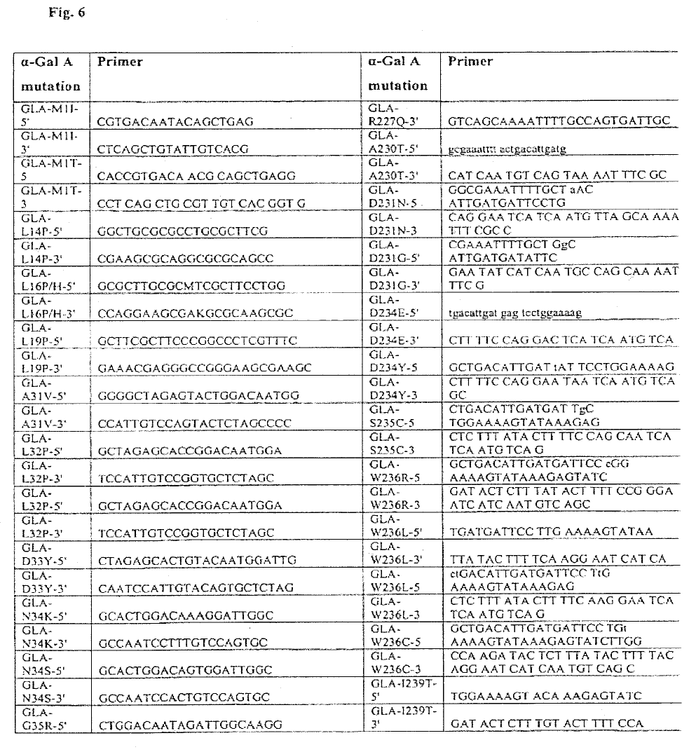

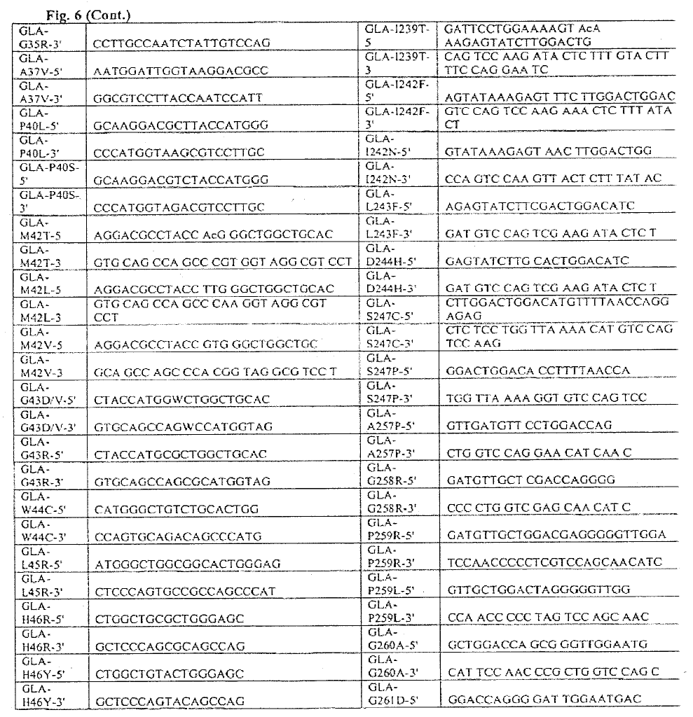

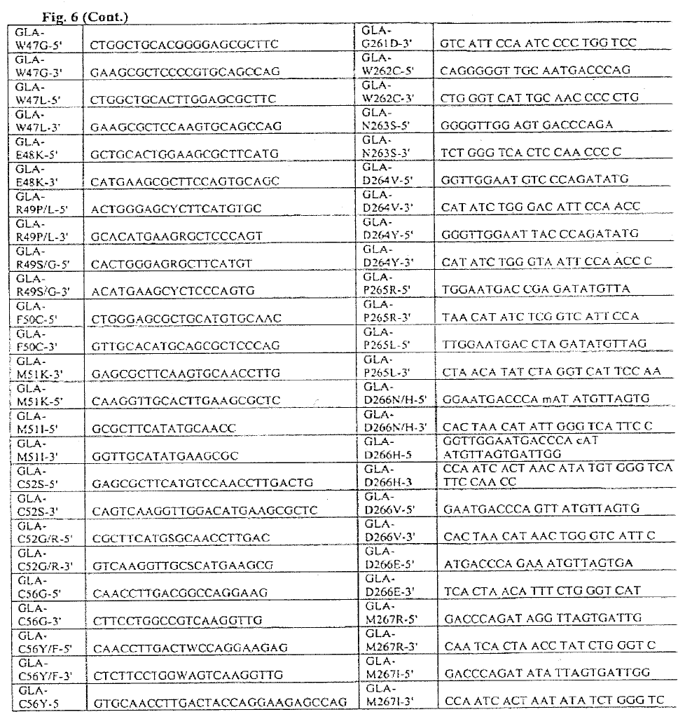

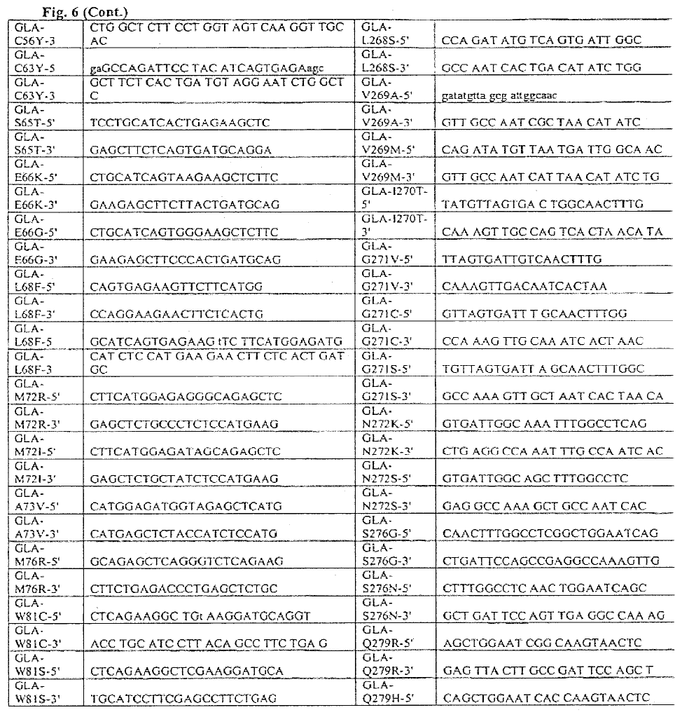

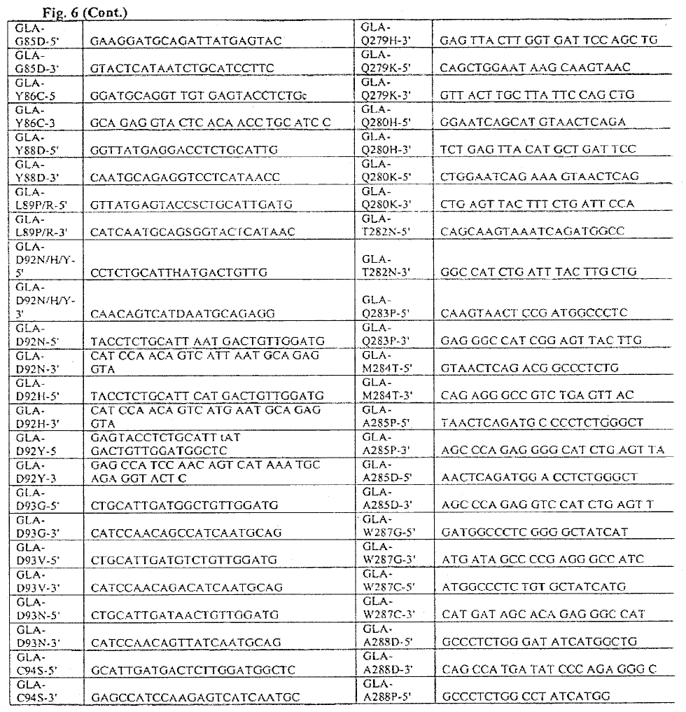

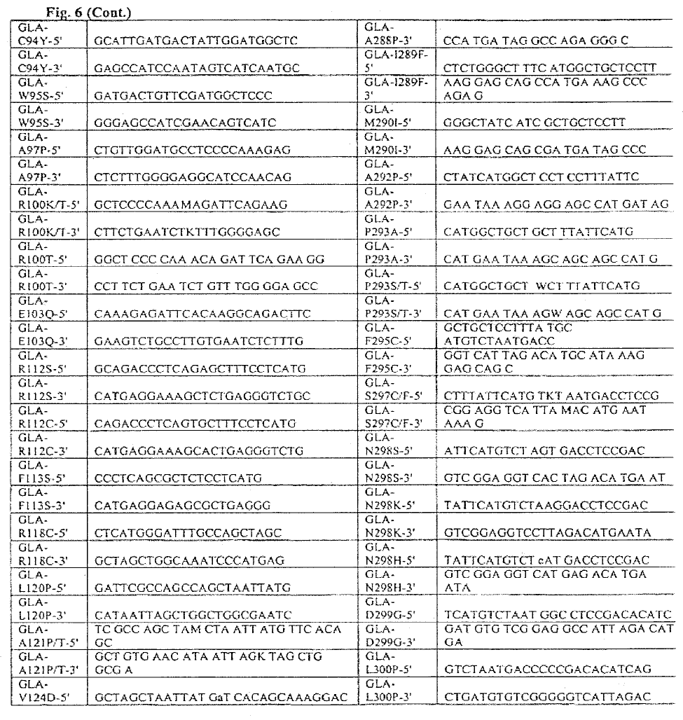

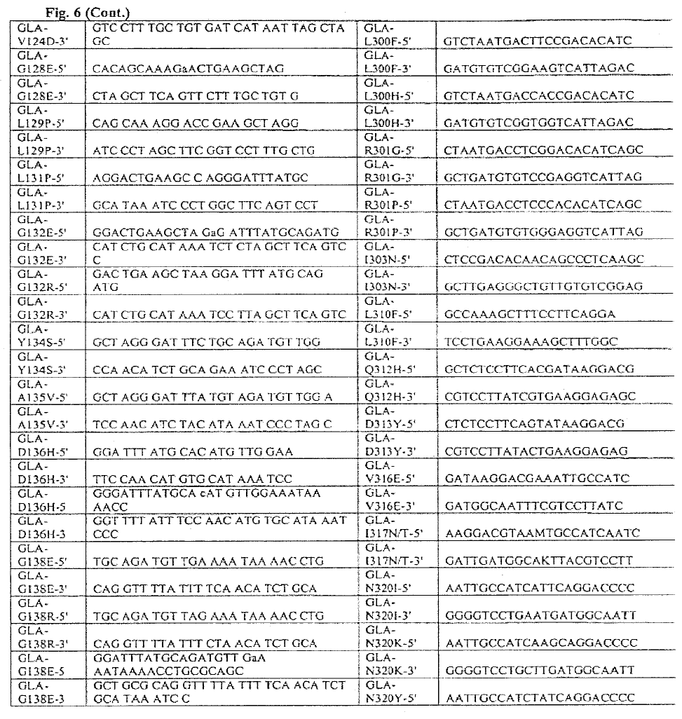

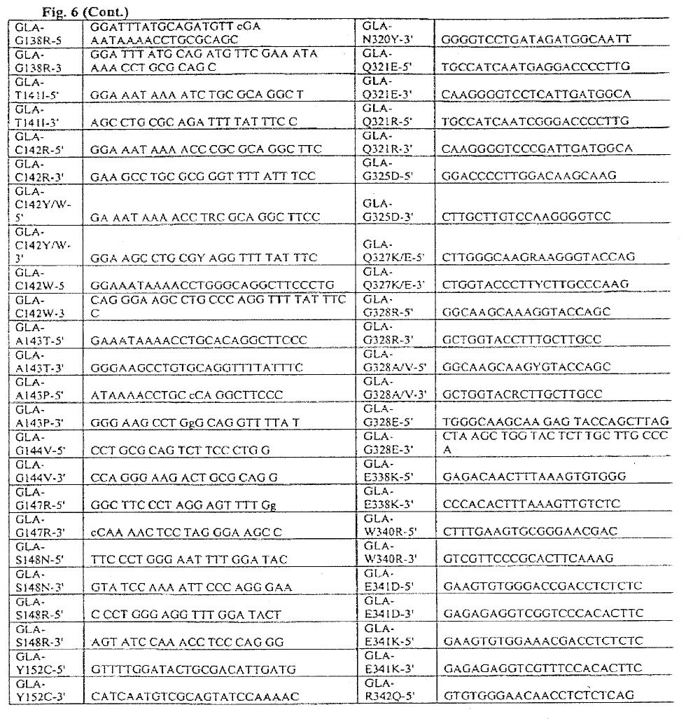

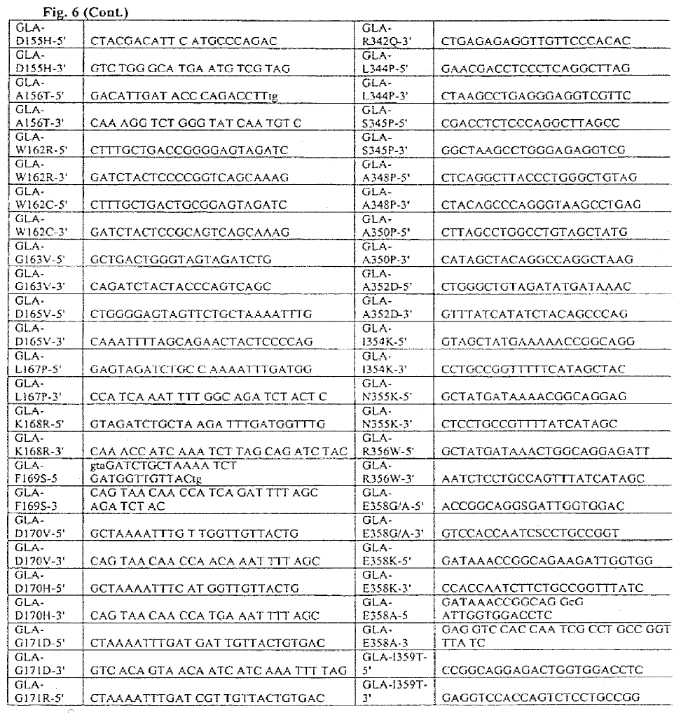

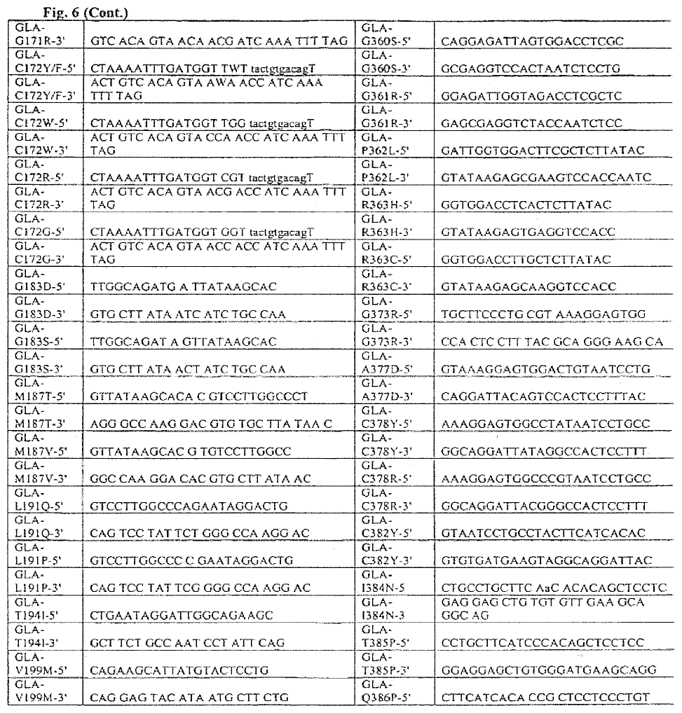

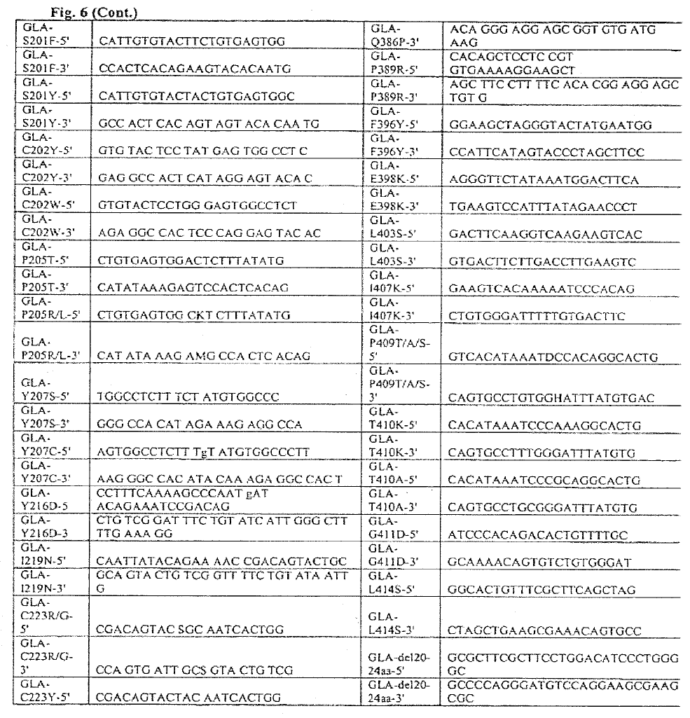

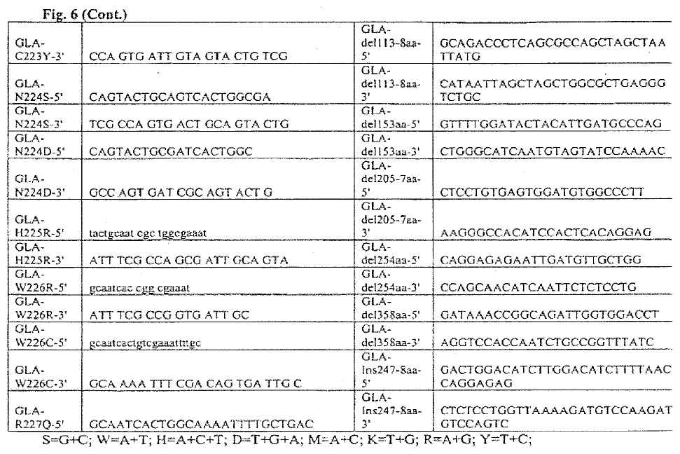

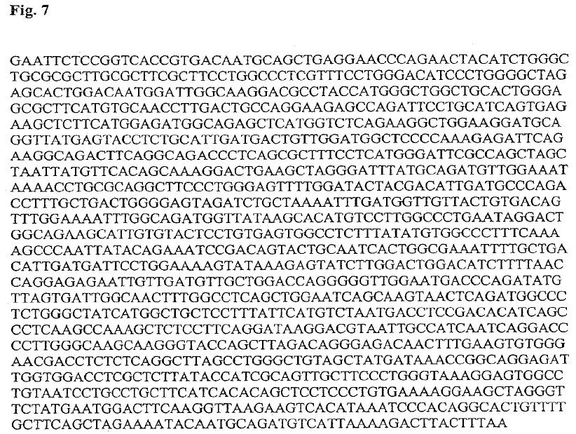

Mutagenesis: All mutations were generated by site-directed mutagenesis following standard molecular biology protocols. To generate point mutations, site-directed mutagenesis was used on the expression vector pcDNA3.1 (Invitrogen) containing human α-GAL A cDNA in-frame. Specific primer pairs were designed containing the desired mutation (FIG. 6). The mutagenesis was performed through the polymerase chain reaction using PfuUltra high-fidelity DNA polymerase (Stratagene) in a thermocycler. Each reaction mixture contained a total volume of 50 μl with the following: 41.6 μl dH₂O, 5.0 μl 10× PfuUltra HF reaction buffer, 0.5 μL Forward-5′-primer (50 uM), 0.5 μl Reverse-3′-primer, 1.0 μl dNTP mix (containing 25 mM each dA, dT, dC, dG), 0.9 μl human GLA in pcDNA3 (2 ng/μl DNA), 0.5 μl PfuUltra HD DNA polymerase. Thermocycler parameter used was the following: i) 94° C. for 30 seconds, ii) 94° C. for 30 seconds, 55-60° C. for 30 seconds, 68° C. for 6 minutes, iii) Repeat (ii) 16 times. Afterwards, 0.5 μl Dpn I (New England Biolabs) was added to each reaction and incubated at 37° C. for 2 hours. A volume of 7.5 μl for each mutagenesis reaction was used to transform DH5α cells (New England Biolabs). Cells were then plated on LB-agar plates with 75 ug/ml ampicillin, and incubated at 37° C. overnight. Bacterial colonies were picked, grown in liquid LB with ampicillin overnight, shaking, at 37° C., and plasmid DNA extracted using QuickLyse Miniprep Kit (Qiagen). Mutants were confirmed by sequencing the full-length human GLA gene. For some of the mutants, human GLA cDNA was contained in the vector plasmid pCXN. Mutagenesis was performed in this vector with the NEB Fusion DNA polymerase. After confirming the mutation through sequencing, the plasmid was digested with EcoRI and subcloned into expression vector pcDNA3.1. Correct orientation was confirmed by digestion with Xho I.

Transient transfection and expression: Transient transfection was carried out in GripTite 293 MSR cells (Invitrogen Corp., Carlsbad, Calif., U.S.A.) using the reagent Fugene HD (Roche). Briefly, cells were seeded in 96-well plates (Costar) at a density of 7.5-10 k cells/well and incubated at 37° C., 5% CO₂ for 24 hours before transfection. Cells were transfected with 0.1 μg DNA and 0.35 μL of Fugene HD reagent per well (DNA:Reagent ratio of 2:7). After transfection with expression constructs containing the specific α-Gal A mutants, cells were incubated again in 37° C., 5% CO₂ for one hour before adding DGJ at 20 nM to 1 mM. Cells were then incubated for 4-5 days before lysis and assay.

α-GAL A Activity Measurement: Cells were washed two times with PBS then incubated in 200 μl fresh media at 37° C., 5% CO₂ for two hours followed by 2 additional PBS washes. After, cells were lysed in 60 μL Lysis Buffer (27 mM sodium citrate/46 mM sodium phosphate dibasic, 0.5% Triton X-100, pH 4.6). Ten μL lysate were added to 50 μL assay buffer (Lysis Buffer without Triton X-100, but containing 6 mM 4-MU-α-D-galactopyranoside (4-MUG) and 117 mM N-acetyl-D-galactosamine (GalNac)), and incubated at 37° C. for 1 hr. Seventy μL Stop Solution (0.4 M glycine, pH 10.8) were then added and fluorescence read on a Victor plate reader (Perkin Elmer) at 355 nm excitation and 460 nm emission. Raw fluorescence counts were background subtracted as defined by counts from substrate solution only. A MicroBCA Protein Assay Kit (Pierce) was used according to manufacturer's instructions to determine protein concentration from 40 μL of cell lysate. A 4-methylumbelliferone (4-MU) standard curve ranging from 30 μM to 1.3 nM was run in parallel for calculation of absolute α-Gal A activity expressed as nmoles/mg protein/hr or further normalized to % of untreated wild type enzyme activity.

Transient transfection and α-Gal A activity measurements were performed in quadruplicates and repeated at least 3 times for each mutation to calculate the average α-Gal A activity at each DGJ concentration. Significant response to DGJ was determined by a two-tailed, paired Student's T-test (p<0.05).

### Results

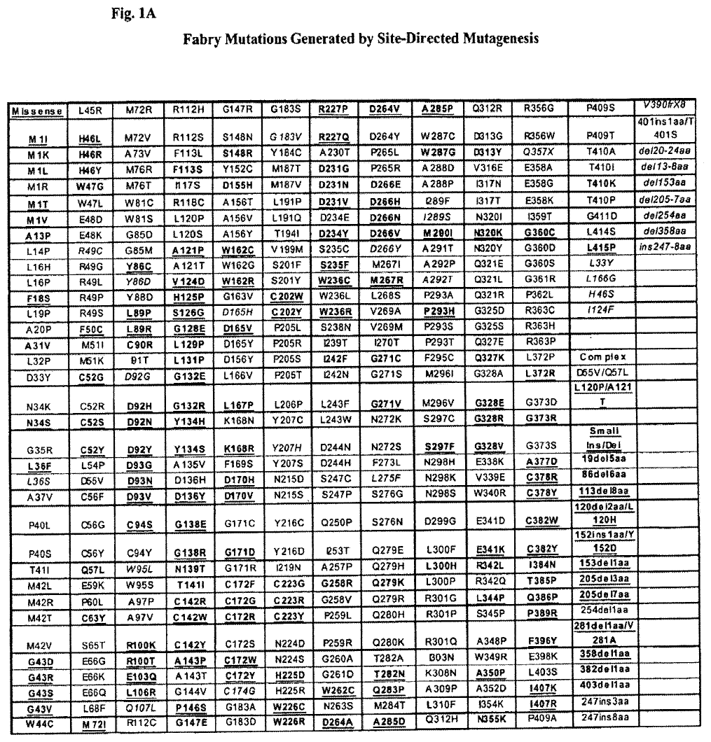

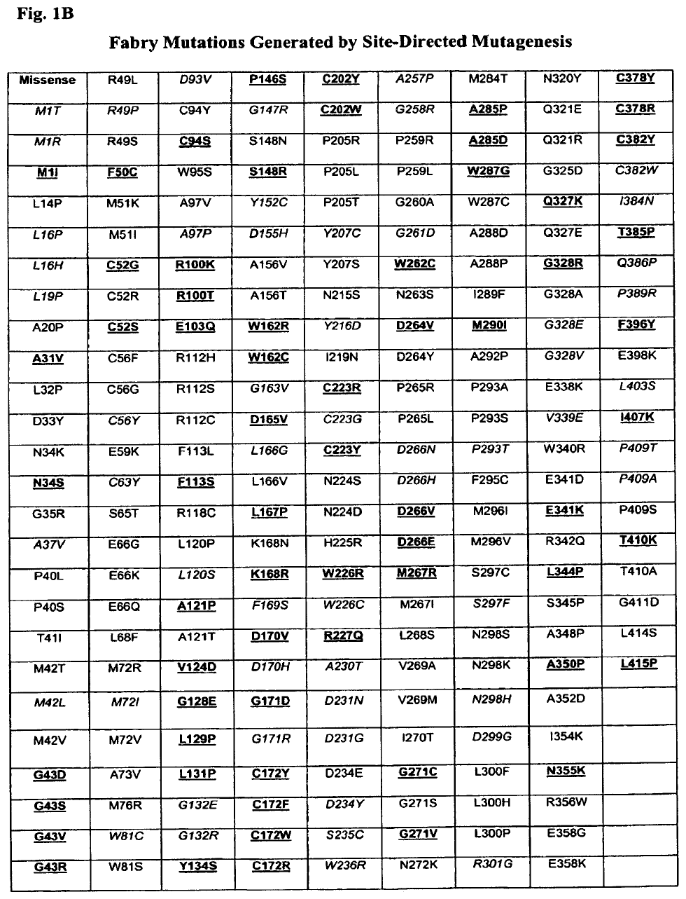

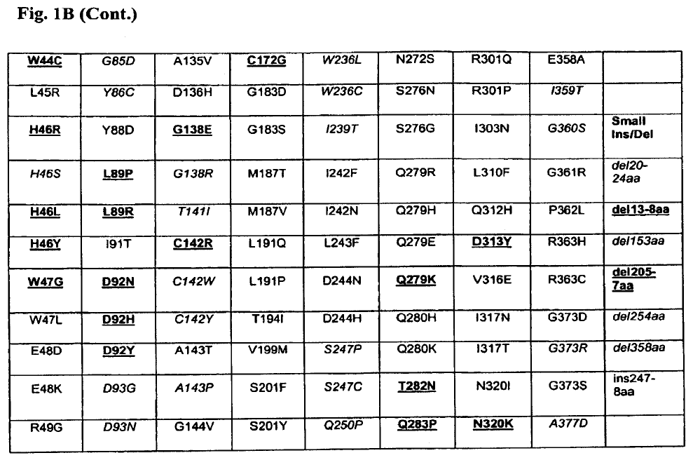

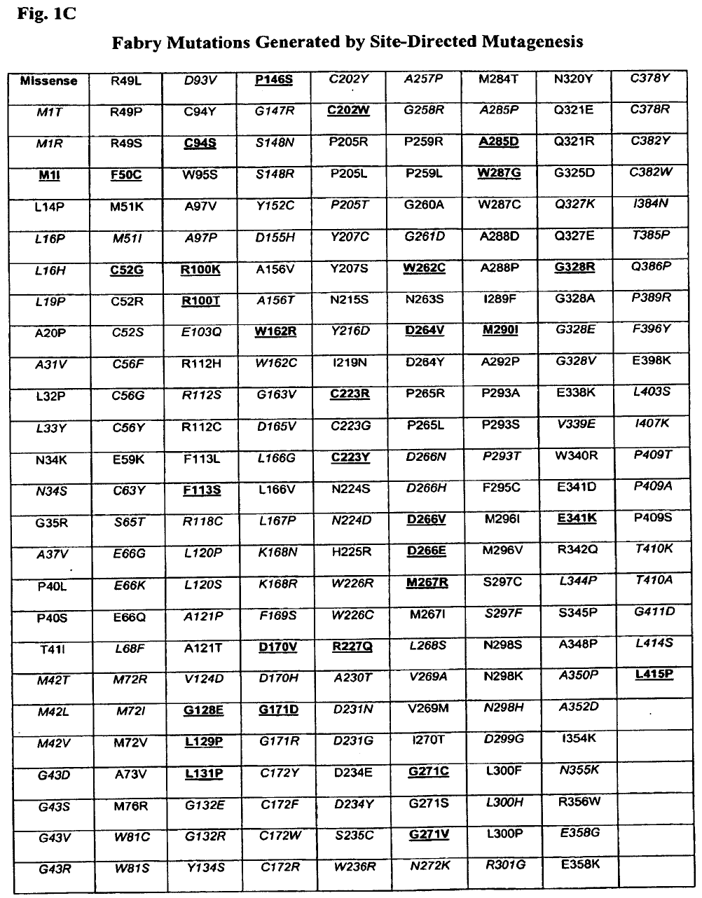

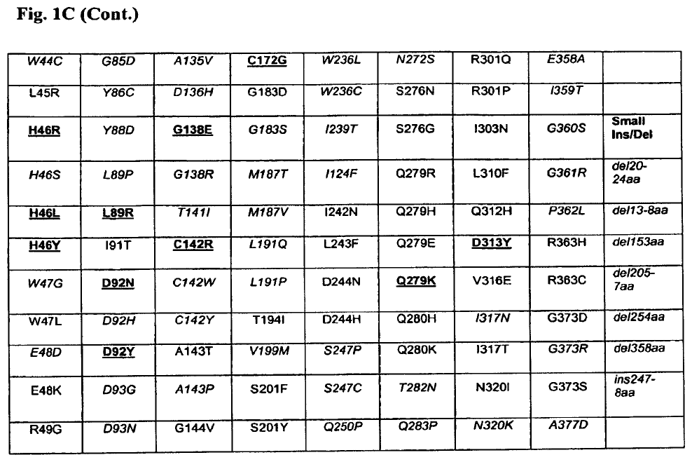

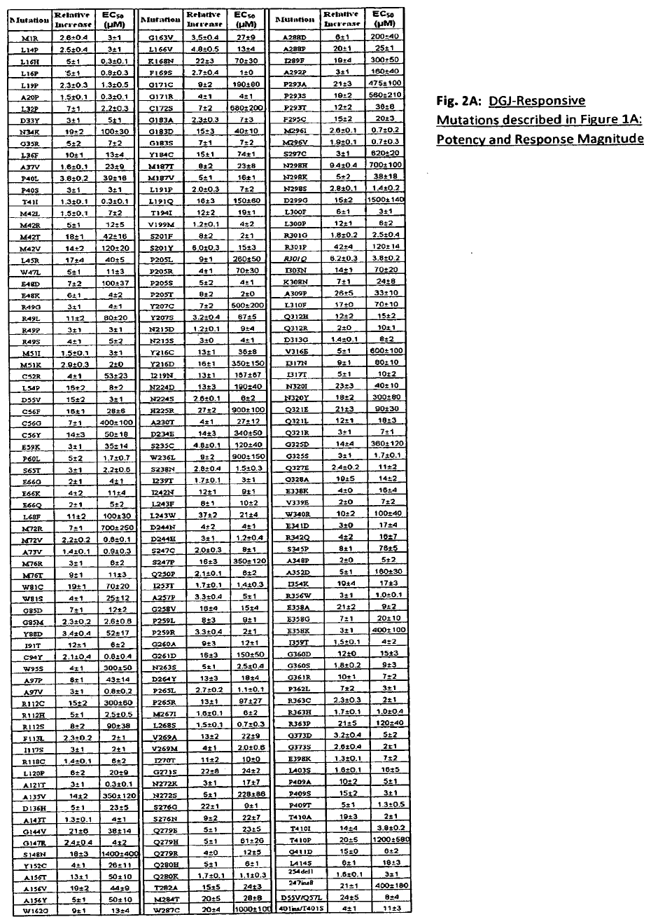

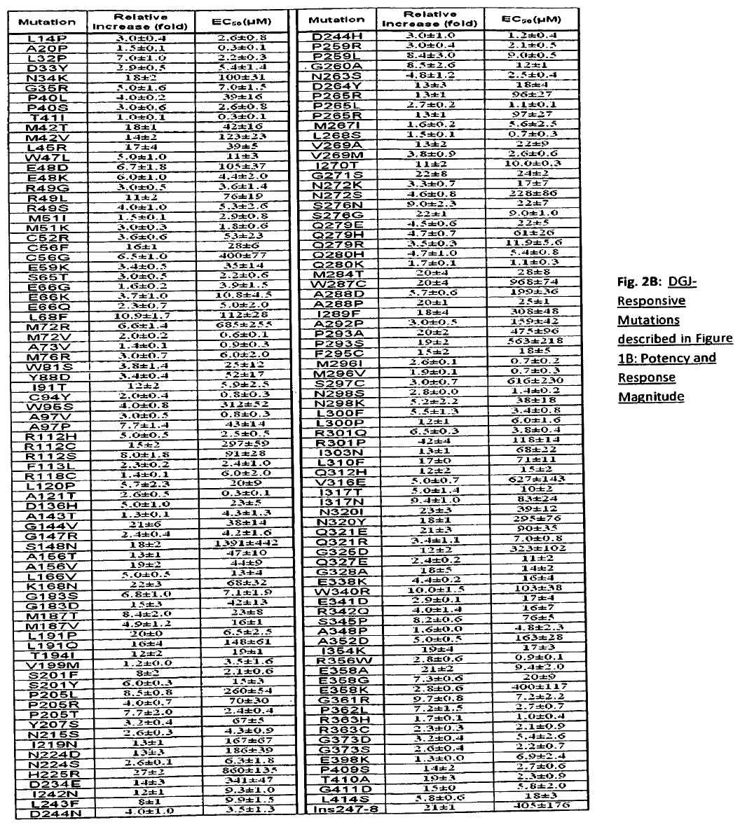

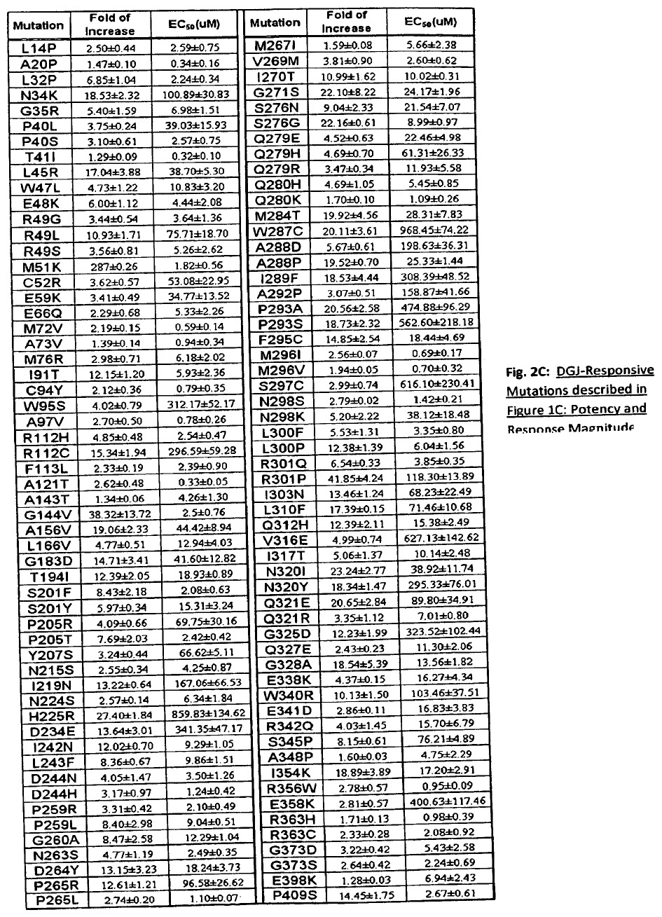

All listed Fabry mutations were generated by site-directed mutagenesis (FIG. 1). Mutations identified in italicized text were not tested, while those identified in plain text were α-Gal A mutants that were responsive to DGJ treatment in the transient transfection assay, and those identified in bold and underscored text were not responsive to DGJ treatment in the transient transfection assay. The magnitude of increase in α-Gal A levels after DGJ treatment and EC50 values are listed for every tested mutation that responded to DGJ treatment (FIG. 2).

α-Gal A activity (expressed as nmol/mg protein/hr of 4-MU released) was measured in lysates prepared from transfected GripTite 293 cells incubated with increasing concentrations of DGJ. A typical concentration-dependent response is shown for L300P and a typical negative response to DGJ is shown for R227Q. Wild type exhibits high baseline activity and does not respond to DGJ in this assay (FIG. 3).

α-Gal A levels were measured in three different assays, reported as percentage of wild type, are compared for each mutation by plotting side by side. The three different assays examined α-Gal A levels in T-cells and lymphoblasts isolated from Fabry patients (for example, see U.S. Ser. No. 11/749,512), as well as in white blood cell (WBC) from DGJ Phase 2 studies

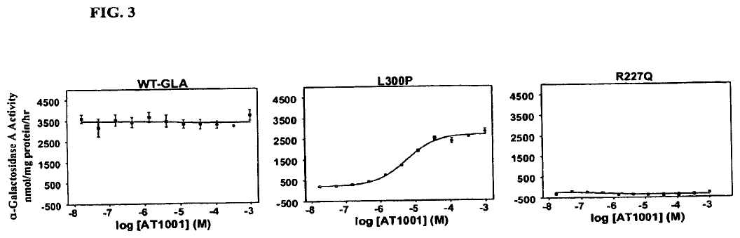

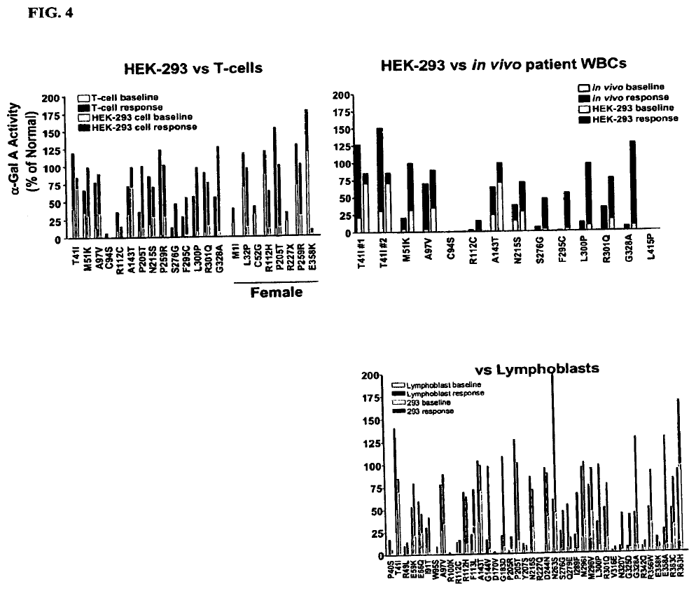

Blank bars indicate basal level (without DGJ treatment) and filled bars indicate the elevated level after DGJ treatment (FIG. 4).

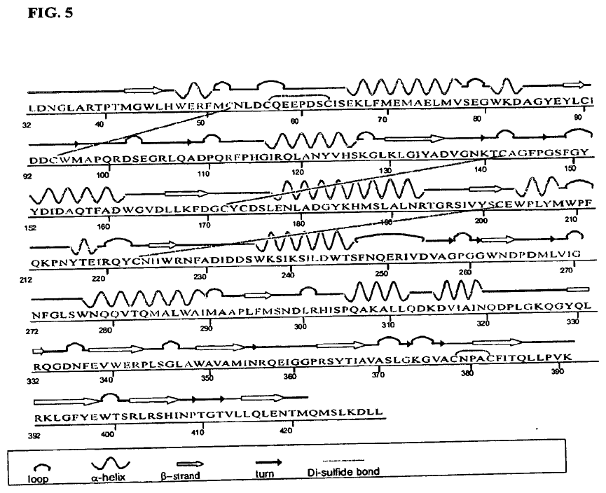

Tested Fabry mutations were illustrated on the α-Gal A secondary structure (FIG. 5). No significant correlation between response and location on the protein sequence of a mutation was observed, suggesting that responsive as well as non-responsive mutations are distributed widely across the entire protein. Text color indicates DGJ response: green=response; red=no response; brown indicates that of the multiple mutations on that same site some responded to DGJ treatment, while others did not.

### Conclusion

These described results are comparable to those obtained from Fabry patient-derived lymphoid or T cells, as well as to the α-Gal A enzyme responses observed in the white blood cells of Fabry patients after oral administration of DGJ in Phase 2 clinical trials.

Thus, the GripTite 293 MSR transient transfection assay is a reliable method for identifying DGJ-responsive mutations and characterizing the magnitude and potency of this response.

Among the responsive mutations identified, the increases in α-Gal A levels by DGJ treatment ranged from 1.3- to 40-fold (2% to 100% wild type), with EC₅₀ values between 200 nM and >100 mM.

DGJ-responsive and non-responsive mutant forms did not appear to be located to particular regions or domains on the α-Gal A protein structure.

### Example 2: Ex Vivo Method for Evaluating Effects of an SPC on Glucocerebrosidase Activity-Prophetic Example

Gaucher disease (GD) is caused by a deficiency of lysosomal glucocerebrosidase (GCase). Deficient GCase activity leads to an accumulation of glucosylceramide (GlcCer) and the development of symptoms such as anemia, thrombocytopenia, hepatosplenomegaly, bone necrosis, infarcts and osteoporosis, and in some cases, neuropathic disease. The specific pharmacological chaperone isofagomine tartrate (IFG) selectively binds and stabilizes mutant (N370S/N370S) GCase in the ER and increases its trafficking to the lysosome.

To evaluate the effects of IFG on different GCase variants, an ex vivo diagnostic assay will be prepared using Cos7 cells in order to ascertain IFG-responsive mutations.

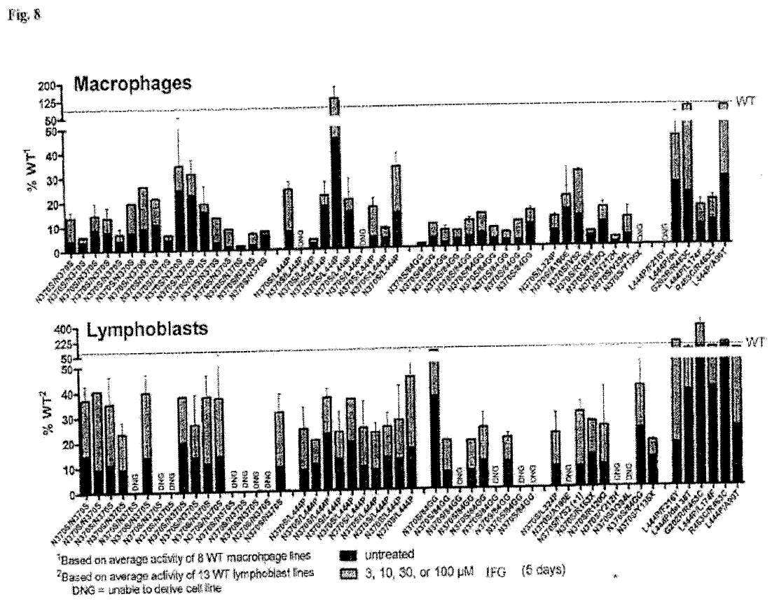

Using the techniques described in Examples 1 and 4, COS-7 cell lines will be prepared that express missense mutations and several in-frame small deletions and insertions by site-directed mutagenesis. Assays will be prepared for all of the mutations listed in the x-axis of FIG. 8. IFG-activity response will be ascertained for each assay according to methods known in the art (sec, e.g., U.S. Pat. No. 6,916,829, which is hereby incorporated by reference).

To determine the correlation of the IFG-response measured in the COS-7 cells to patient-derived cells, IFG-activity response was also measured in Patient-Derived Macrophages and Lymphoblasts. Macrophages were successfully derived from 46 of 63 patients and incubation with IFG (3, 10, 30 or 100 μM) for 5 days increased GCase levels in macrophages from 42 of 46 patients (mean=2.3-fold; range: 1.1- to 6.5-fold). Residual activity levels and response to IFG was more consistent for the same genotypes when measured in lymphoblasts compared to macrophages, potentially due to the variability in macrophage viability between different patients. The results are shown in FIG. 8.

The response to IFG for the patient-derived cells will be compared to the results obtained in the Cos7 cell line.

### Example 3: In Vivo Effect of an SPC on α-GAL A Activity in Skin, Heart, Kidney and Plasma

To determine if increased mutant α-Gal A levels translate to increased α-Gal A activity in situ, the effect of DGJ administration on tissue GL-3 levels was investigated in vivo in hR301Q α-Gal A Ty/KO mice.

Eight-week old male hR301Q α-Gal A Tg/KO mice were treated for 4 weeks with 300 mg/kg DGJ in drinking water either daily or less frequently (4 days ON/3 days OFF). After dosing, lysates were prepared from skin, heart, kidney, and plasma by homogenizing ˜50 mg tissue in Lysis Buffer (sec above). 20 μL lysate were mixed with 50 μL of substrate (as detailed above). Reaction mixtures were incubated at 37° C. for 1 hr. After, 70 μL Stop Solution were added and fluorescence was read on a Victor plate reader as described above. Enzyme activity in the lysates was background subtracted, and normalized for protein concentration. A 4-MU standard curve was run for conversion of fluorescence data to absolute α-Gal A activity expressed as nmol/mg protein/hr.

Tissue samples were washed free of blood, weighed and homogenized with a solvent system in a FastPrep® system. Homogenate was then extracted using Solid Phase Extraction on a C18 cartridge. The eluent was evaporated and reconstituted prior to injection onto a LC-MS/MS system. Twelve GL-3 isoforms were measured using positive ESI-MS/MS. LC separation was achieved on 00839a Zorbax C18 column.

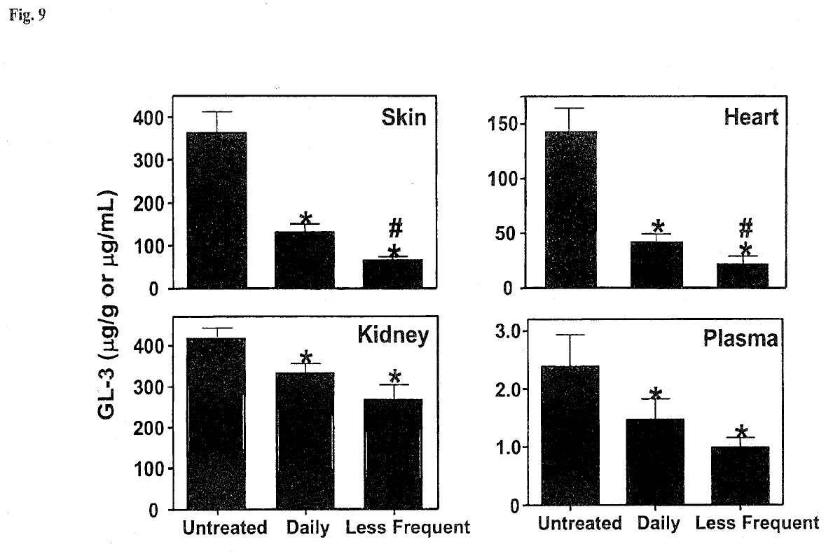

Significant decreases in GL-3 levels were seen with daily and less frequent DGJ dosing in skin, heart, kidney, and plasma (FIG. 9). A trend of greater reduction in GL-3 levels was seen in multiple tissues and plasma with less frequent DGJ dosing. Collectively, these results indicate that DGJ merits further evaluation for the treatment of patients with Fabry disease.

### Example 4: Identification of Pompe Disease-Causing Mutations That are Responsive to the Pharmacological Chaperone DNJ

Pompe disease is caused by deficient acid alpha glucosidase (GAA) activity which impairs lysosomal glycogen metabolism. The enzyme deficiency leads to lysosomal glycogen accumulation and results in progressive skeletal muscle weakness, reduced cardiac function, respiratory insufficiency, and CNS impairment at late stages of disease. Genetic mutations in the GAA gene result in either lower expression or produce mutant forms of the enzyme with altered stability, and/or biological activity ultimately leading to disease. Pharmacological chaperones represent a promising new therapeutic approach for the treatment of genetic diseases.

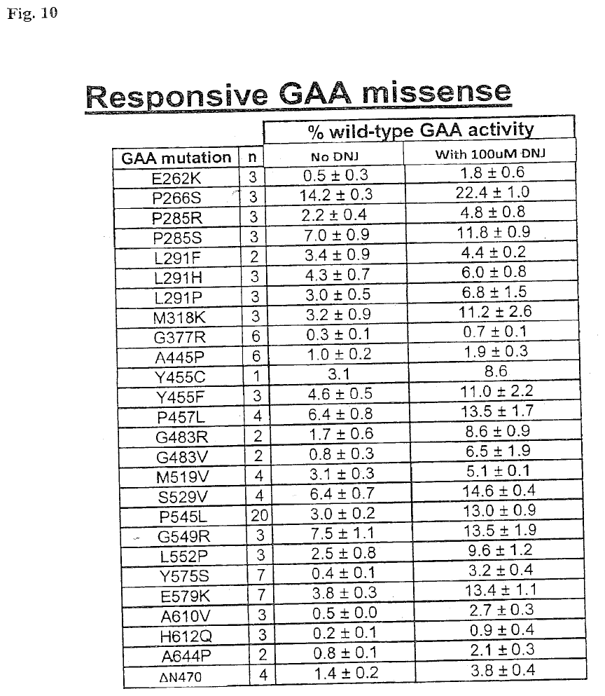

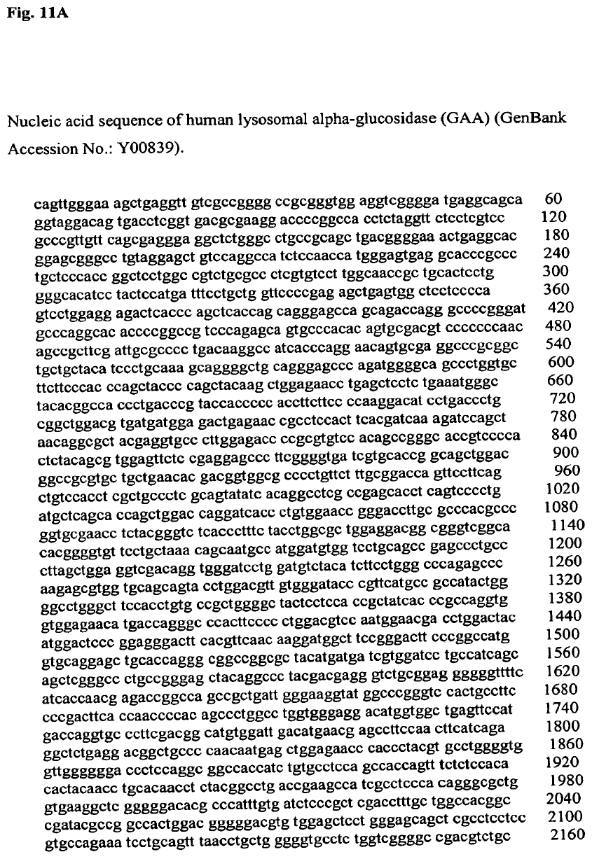

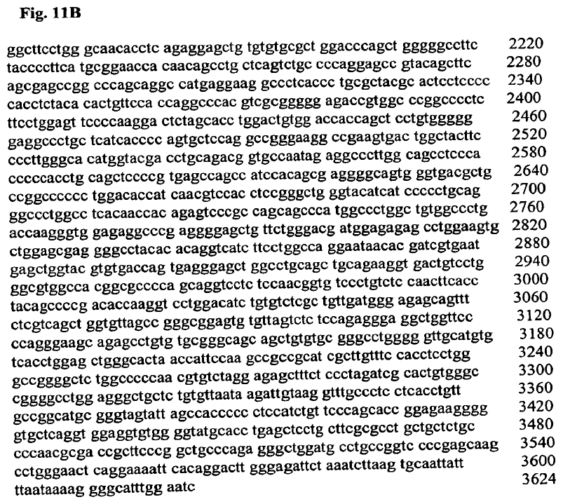

To evaluate the effects of DNJ on different GAA variants, an in vitro diagnostic assay was prepared using COS-7 and HEK-293 cells in order to ascertain DNJ-responsive mutations (FIGS. 10, 12 and 14)

A site-directed mutagenesis approach was employed to introduce specific mutations into the complementary DNA (cDNA) encoding wild-type human acid α-glucosidase (GAA). The initial wild-type GAA DNA construct was generated by subcloning the GAA coding region from cDNA clone 5739991 (Invitrogen) into the pcDNA6N5-HisA mammalian expression vector (Initrogen). The resultant DNA construct (designated as wild-type GAA cDNA) was used as the DNA template for subsequent mutagenesis. These missense, small insertion or deletion mutations are cited in the Erasmus database and known to be associated with type 2 glycogen storage disorder (GSD II), also known as Pompe disease. Briefly, wild-type GAA cDNA was PCR-amplified using mutagenic primers to obtain plasmid DNA with the desired mutation These mutations were confirmed by DNA sequencing prior to protein expression in cells.

COS-7 cells (derived from green monkey embryonic kidney cells) were aseptically seeded in 12-well tissue culture plates at a cell density of ˜1.4×10⁵ cells per well in 3 ml of Dulbecco's Modified Essential Medium (DMEM) containing 10% (v/v) fetal bovine serum and grown overnight at 37° C. in a humidified 5% CO₂ atmosphere. On the following day, the cells (typically 60-80% confluent) were transfected with 0.75 μg of the individual DNA construct via a lipid transfection reagent such as FUGENE HD (Roche) according the manufacturer's instructions. Two wells were transfected with each DNA construct such that one well was incubated with DNJ (typically 0 μM, 20 μM, 50 μM or 100 μM) while an equivalent volume of PBS was added to the other well. Two additional wells were transfected with the empty vector (no GAA cDNA) and incubated with or without DNJ to serve as the background control for endogenous monkey GAA expression. Similarly, 2 additional wells were transfected with the wild-type human GAA cDNA and incubated with or without DNJ to serve as the positive control. All samples were incubated for ˜48 hrs at 37° C. in a humidified 5% CO₂ atmosphere.

After the 48-hour incubation period, the spent media was removed and the cells were washed with PBS and then incubated with fresh 1-2 ml DMEM medium for 3 hours at 37° C. in a humidified 5% CO₂ atmosphere. The medium was subsequently removed and cells were immediately washed with PBS and lysed with 200 μl of Lysis Buffer (25 mM Bis-Tris (pH 6.5), 150 mM NaCl, 1% (v/v) Triton X-100) containing a cocktail of protease inhibitors. The cell culture plate were then gently swirled on a rotating orbital shaker apparatus for 10 min at room temperature for complete cell lysis. The resultant cell lysates were transferred to clean 1.5 ml microcentrifuge tubes and spun at 20,000×g for 10 mM to pellet cellular debris. Approximately 175 μl of each supernatant sample was then transferred to a 1.5 ml fresh microcentrifuge tube. This cell lysate was used for all subsequent assays including GAA enzyme activity, total protein concentration determination, and Western blotting.

Residual GAA enzyme activity was determined for each transiently-expressed GAA using a fluorogenic 4-methylumbeliferyl-α-glucopyranoside (4-MU-α-glucose) substrate (Sigma). Briefly, 10 μl of each cell lysate was assayed (in triplicate) in a 100 μl reaction in 96-well clear bottom black plates using 3 mM 4-MU-α-glucose and 50 mM KOAc (pH 4.0). The transiently-expressed wild-type GAA sample was diluted 20-fold with Lysis Buffer to ensure that the enzymatic reaction is maintained within the linear range of the instrument. The enzyme reactions were performed at 37° C. for 1 hour and terminated by the addition of 50 μl of 500 mM Na₂CO₃ (pH 10.5). The assay was then read in a fluorescence plate reader (using 355 nm excitation/460 nm emission) to quantitate the amount of GAA-dependent 4-MU fluorescence liberated. The GAA enzyme activity was then extrapolated from a free 4-MU standard curve after subtracting the background fluorescence (i.e., empty vector control).

Twenty five microliters of each cell lysate was used in a parallel assay to determine the total cellular protein concentration using the bicinchoninic acid (BCA) protein assay (Pierce) according to the manufacturer's protocol. The total cellular protein concentration was extrapolated from a bovine serum albumin (BSA) standard curve.

The GAA enzyme activity for each sample was normalized to the total cellular protein concentration and expressed as the nmoles of 4-MU released/mg total protein/hr to define the GAA specific activity. The resultant GAA specific activity after DNJ treatment was compared to GAA enzyme activity of the corresponding untreated sample to determine whether a specific GAA mutant responds to DNJ.

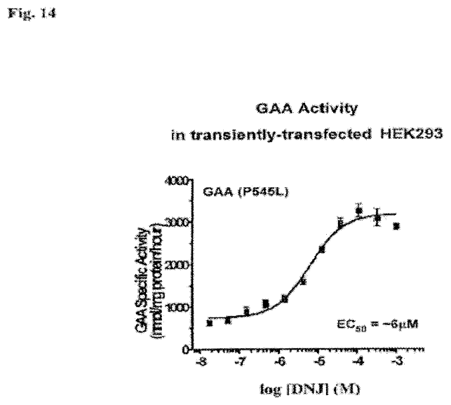

For a single HEK-293 cell line transfected with the GAA mutation, P545L, the DNJ EC₅₀ was also determined (FIG. 14).

To determine the correlation of the DNJ-response measured in the COS-7 cells to patient-derived cells, DNJ-activity response was also measured ex vivo in Patient-Derived Macrophages and Lymphoblasts.

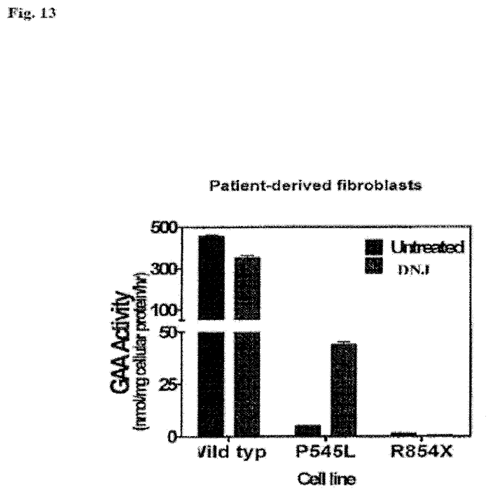

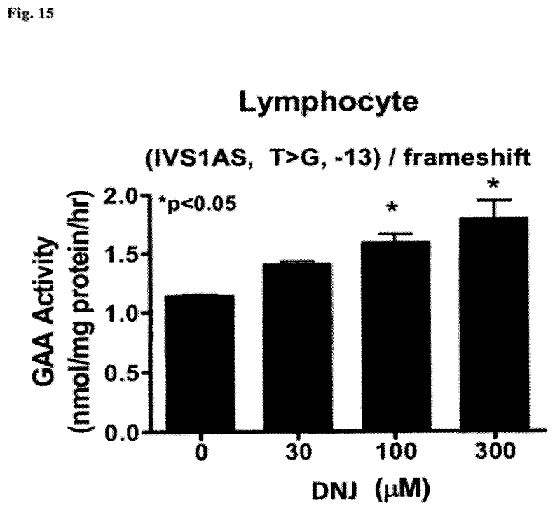

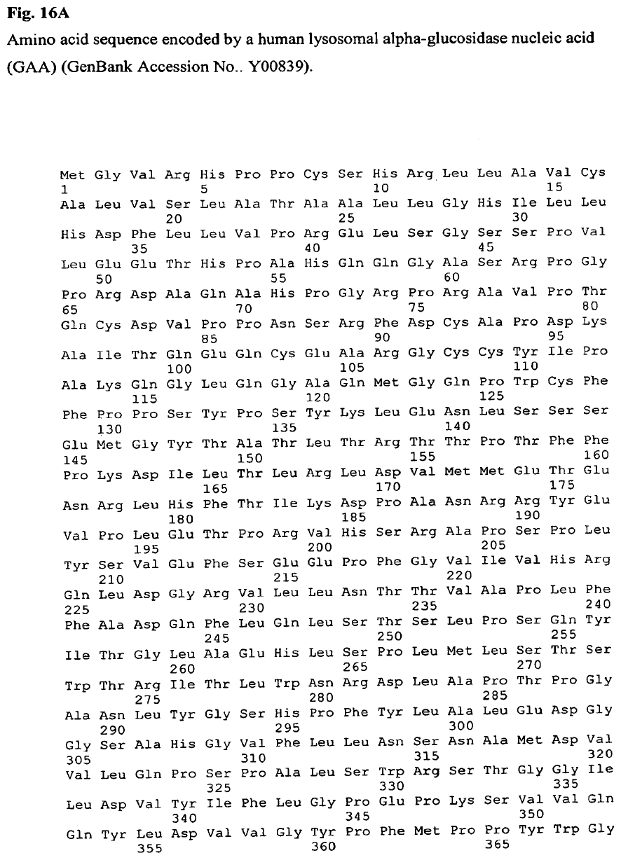

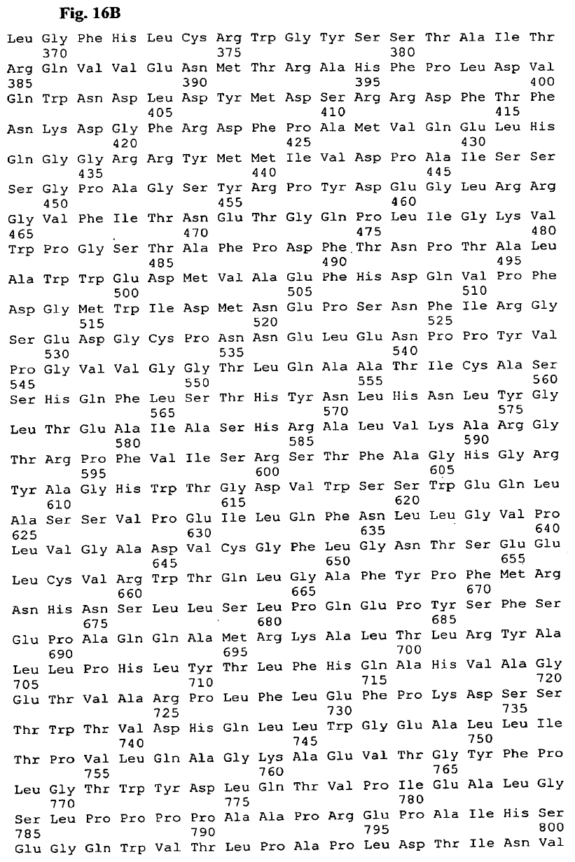

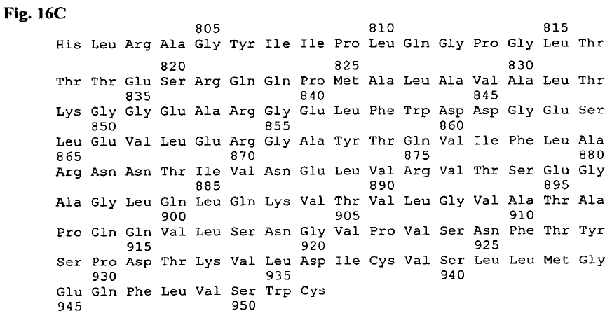

Fibroblast and lymphocyte cell lines derived from Pompe patients were also generated as previously described (see U.S. Ser. No. 11/749,512). Fibroblast cell lines were derived from patients homozygous for the P545L or R854X GAA mutations (FIG. 13). Lymphocyte cell lines were derived from patients heterozygous for the (IVS1AS, T>G, -13) GAA splicing defect and GAA frameshift mutation (FIG. 15).

GAA activity was measured in the lymphocyte cell lines following incubation in 0 μM, 30 μM, 100 μM, or 300 μM DNJ (FIG. 15). GAA activity was also measured in the fibroblast cell lines following DNJ incubation (FIG. 13).

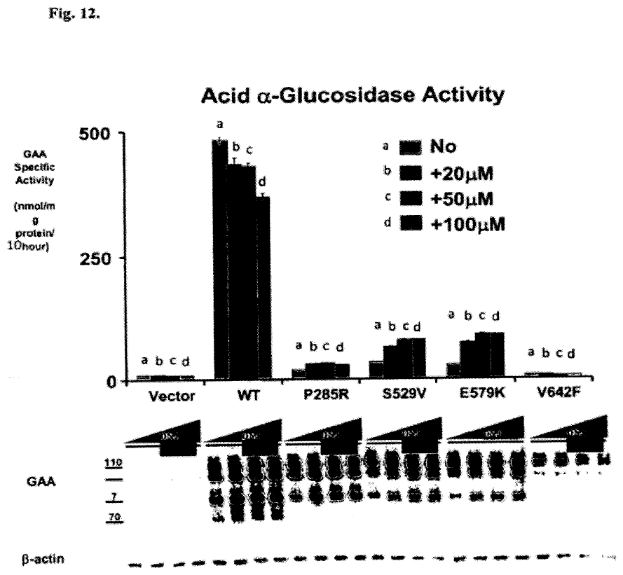

In this study, the pharmacological chaperone 1-deoxynojirimycin-HCl (DNJ) is shown to bind mutant GAA and increase its activity. In Pompe patient-derived fibroblasts (FIG. 13) and lymphocytes (FIG. 15), as well as in transiently transfected COS-7 (FIGS. 10 and 12) or HEK-293 (FIG. 14) cells expressing certain GAA missense mutations, DNJ significantly increases GAA levels.

DNJ increased GAA activity for 26 mutations (FIG. 10) out of 131 mutants tested (data not shown). In addition to increasing the activity of these mutant GAA's, DNJ also promoted processing of GAA to the 95/76/70 kDa forms.

Furthermore, dose-dependent increases in GAA activity was observed in patient-derived lymphocytes containing the common IVS1AS, T>G, -13 splicing defect in one allele and a frameshift mutation in the second allele (FIG. 15).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, GenBank Accession Numbers, and protocols are cited throughout this application, the disclosures of which are incorporated herein by reference in their entireties for all purpose.

1-8. (canceled)

9. A method of treating a patient diagnosed with Fabry disease which comprises administering to the patient a therapeutically effective dose of 1-deoxygalactonorjirimycin or a salt thereof, wherein the patient is identified as having a responsive mutation in α-galactosidase A as determined by a human embryonic kidney (HEK-293) cell assay.
